SEQUENCE LISTING (2 sequences)

SEQ ID NO: 1 - DNA, 20 bp, synthetic construct
  misc_feature 1..20: note = Hypothetical DNA molecule
  source 1..20: mol_type = other DNA
atcgatcgctagatccatcg

SEQ ID NO: 2 - DNA, 20 bp, synthetic construct
  misc_feature 1..20: note = Hypothetical DNA molecule
  source 1..20: mol_type = other DNA
atcgattgctagatccattg

(19) United States
(12) Patent Application Publication
(10) Pub. No.: US 2026/0250778 A1
(43) Pub. Date: Aug. 27, 2026

(54) SYSTEMS AND METHODS FOR DIAGNOSING A CONDITION USING ON-TARGET AND OFF-TARGET SEQUENCING DATA

(71) Applicant: GRAIL, Inc., Menlo Park, CA (US)

(72) Inventors: Anton Valouev, Palo Alto, CA (US); Jing Xiang, San Carlos, CA (US); Collin Melton, Menlo Park, CA (US)

(73) Assignee: GRAIL, Inc., Menlo Park, CA (US)

(21) Appl. No.: 19/379,553

(22) Filed: Nov. 4, 2025

Related U.S. Application Data
(63) Continuation of application No. 17/023,185, filed on Sep. 16, 2020, now abandoned.
(60) Provisional application No. 62/904,455, filed on Sep. 23, 2019.

(51) Int. Cl. C12Q 1/6886; C12Q 1/6827; C12Q 1/6837; C12Q 1/6858; G16B 20/10; G16B 25/10; G16B 40/20; G16B 50/30

(52) U.S. Cl. CPC C12Q 1/6886; C12Q 1/6827; C12Q 1/6837; C12Q 1/6858; G16B 20/10; G16B 25/10; G16B 40/20; G16B 50/30; C12Q 2600/154; C12Q 2600/156

(57) ABSTRACT

Systems and methods for determining whether a subject has a disease condition in a set of disease conditions are provided. The method includes obtaining a test dataset that comprises a first plurality of bin values obtained for a first plurality of bins collectively representing a first portion of a reference genome, and a second plurality of bin values obtained for a second plurality of bins collectively representing a second portion of the reference genome. The first and second plurality of bin values are derived from a targeted sequencing of a plurality of nucleic acids that are enriched using a plurality of probes. A plurality of copy number values are determined from the first and second plurality of bin values. The copy number values are inputted into a trained classifier, thereby determining whether the subject has a disease condition.

### CROSS REFERENCE TO RELATED APPLICATIONS

This patent application is a continuation of U.S. patent application Ser. No. 17/023,185, filed on Sep. 16, 2020, which claims the benefit of priority to U.S. Provisional Patent Application No. 62/904,455, filed on Sep. 23, 2019.

### TECHNICAL FIELD

This disclosure relates to improvements in targeted sequencing technologies where probes are used to target specific regions of a genome prior to sequencing reactions. The disclosure describes using sequencing data from on-target, off-target genomic regions, or a combination of on-target and off-target genomic regions to determine whether a subject has a disease condition, in particular, a cancer condition.

### SEQUENCE LISTING

The instant application contains a sequence listing which has been submitted electronically in XML format and is hereby incorporated by reference in its entirety. The XML copy was created on Nov. 4, 2025, is named “Sequence_Listing.xml”, and is 3,666 bytes in size.

### BACKGROUND

Despite advances in cancer diagnosis and treatment, cancer remains one of the worst diseases that plague the modern world. An important component in addressing cancer is an early and accurate diagnosis. Mistakes in cancer diagnosing can have devastating effects. Thus, incorrect diagnosis, for instance a positive diagnosis when in fact cancer is not present, may result in unnecessary treatment and even surgery, which causes patient suffering and is a waste of time and resources. Correspondingly, a missed diagnosis is undesirable and may lead to loss of life.

Diagnosing a type of a cancer is important for selection and delivery of proper treatment. Also, proper knowledge of cancer stage is important for treatment selection and for monitoring treatment and recovery progress.

Misdiagnosis can occur because cancer is not a single, easily detectable condition, but a complex disease with various molecular alterations that manifest in many different ways. Cells mutate and divide at different rates, new cell types appear, and various, typically uncontrollable, changes occur. A cancerous tissue may have different types of cells that are characteristic of different cancer stages and grades.

The standard approaches to cancer diagnosis include tissue pathology analysis and imaging. Furthermore, due to the increasing knowledge of the molecular basis for cancer and the rapid development of next generation sequencing (NGS) techniques, genomic testing is becoming more widely used. NGS techniques are also advancing the study of early molecular alterations involved in cancer development in tissues and body fluids. Large scale sequencing technologies, including NGS, have afforded the opportunity to achieve sequencing at costs that are less than one U.S. dollar per million bases, and in fact costs of less than ten U.S. cents per million bases have been realized.

Cells can release DNA into the bloodstream, which is referred to as circulating cell-free DNA (cfDNA). Such cells can be found in serum, plasma, urine, and other body fluids (Chan et al., 2003, Ann Clin Biochem. 40(Pt 2):122-130). As such, specific genetic and epigenetic alterations associated with cancer are found in plasma, serum, and urine cfDNA. It has been demonstrated that such alterations can potentially be used as diagnostic biomarkers for several classes of cancers (see, Salvi et al., 2016, Onco Targets Ther. 9, pp. 6549-6559). Thus, cfDNA represents a “liquid biopsy” which is a representation, in circulation, of a specific disease, which may include a tumor (see, De Mattos-Arruda and Caldas, 2016, Mol Oncol. 10(3), pp. 464-474). Such a “liquid biopsy” represents a potential non-invasive method of screening for a variety of cancers. In other words, the liquid biopsy, from the circulatory system, provides a representation of an underlying tumor since the tumor sheds cells into the circulatory system.

The existence of cfDNA was demonstrated by Mandel and Metais decades ago (Mandel and Metais, 1948, C R Seances Soc Biol Fil. 142(3-4), pp. 241-243). cfDNA originates from necrotic or apoptotic cells, and it is generally released by all types of cells. Stroun et al. further showed that specific cancer alterations could be found in the cfDNA of patients (see, Stroun et al., 1989 Oncology 1989 46(5), pp. 318-322). A number of subsequent articles confirmed that cfDNA contains specific tumor-related alterations, such as mutations, methylation, and copy number variations (CNVs), thus confirming the existence of circulating tumor DNA (ctDNA) (see Goessl et al., 2000 Cancer Res. 60(21):5941-5945 and Frenel et al., 2015, Clin Cancer Res. 21(20), pp. 4586-4596).

cfDNA in plasma or serum is well characterized, while urine cfDNA (ucfDNA) has been traditionally less characterized. However, recent studies demonstrated that ucfDNA could also be a promising source of biomarkers (e.g., Casadio et al., 2013, Urol Oncol. 31(8), pp. 1744-1750).

In blood, apoptosis is a frequent event that determines the amount of cfDNA. In cancer patients, however, the amount of cfDNA seems to be also influenced by necrosis (see, Hao et al., 2014, Br J Cancer 111(8), pp. 1482-1489 and Zonta et al., 2015 Adv Clin Chem. 70, pp. 197-246). Since apoptosis seems to be the main release mechanism, circulating cfDNA has a size distribution that reveals an enrichment in short fragments of about 167 bp, (see, Heitzer et al., 2015, Clin Chem. 61(1), pp. 112-123 and Lo et al., 2010, Sci Transl Med. 2(61), 61ra91) corresponding to nucleosomes generated by apoptotic cells.

The amount of circulating cfDNA in serum and plasma has been shown to be significantly higher in patients with tumors than in healthy controls, especially in those with advanced-stage tumors than in early-stage tumors (see Sozzi et al., 2003, J Clin Oncol. 21(21), pp. 3902-3908, Kim et al., 2014, Ann Surg Treat Res. 86(3), pp. 136-142; and Shao et al., 2015, Oncol Lett. 10(6), p. 3478-3482). The variability of the amount of circulating cfDNA is higher in cancer patients than in healthy individuals, (see, Heitzer et al., 2013, Int J Cancer. 133(2), pp. 346-356) and the amount of circulating cfDNA is influenced by several physiological and pathological conditions, including proinflammatory diseases (see, Raptis and Menard, 1980, J Clin Invest. 66(6), pp. 1391-1399, and Shapiro et al., 1983, Cancer 51(11), pp. 2116-2120).

Furthermore, methylation status and other epigenetic modifications are known to be correlated with the presence of some disease conditions such as cancer (see, Jones, 2002, Oncogene 21, pp. 5358-5360). And specific patterns of methylation have been determined to be associated with particular cancer conditions (see Paska and Hudler, 2015, Biochemia Medica 25(2), pp. 161-176). Warton and Samimi have demonstrated that methylation patterns can be observed even in cell free DNA (Warton and Samimi, 2015, Front Mol Biosci, 2(13) doi: 10.3389/fmolb.2015.00013).

Existing techniques for acquiring and processing genomic data, including sequencing data from circulating cfDNA, for cancer diagnosis include various computational approaches that make use of powerful computer technology. Nevertheless, despite the widespread efforts, many existing approaches lack the ability to diagnose cancer with the precision that is suitable for application to patient diagnosis in medical practice.

Thus, given the promise of sequencing data from circulating cfDNA, as well as other forms of genotypic data, as a diagnostic indicator, improved ways of using such data to identify a disease condition (e.g., a cancer condition) in subjects are needed in the art.

### SUMMARY

The present disclosure can improve the field of cancer diagnosis by providing techniques that make use of genomic information found in so-called “on-target” regions and genomic information found in so-called “off-target” regions. The “on-target” regions can be certain regions of a reference genome that correspond to and can be enriched by a series of probes targeting such regions before sequencing reactions take place, and the “off-target” regions can be genomic regions that can substantially differ from the on-target regions. As disclosed herein, the terms “on-target genomic regions” and “on-target regions” can be used interchangeably. Similarly, the terms “off-target genomic regions” and “off-target regions” can be used interchangeably.

Copy number values, which are one of the indicators of genomic variations present in both on-target and off-target regions, can be used to determine whether a subject has a disease condition. Accordingly, in some embodiments, measures of copy number instability, referred to herein as copy number values, are calculated for both on-target regions and off-target regions, and the copy number values are used to determine whether a subject has or does not have a disease or condition (e.g., cancer) and a type of that condition. In some embodiments, combining on-target and off-target data improves the precision and efficacy of a classification of a disease or a non-disease. Thus, the techniques described in the present disclosure can allow for the use of a larger amount of data and for the use of signals in genomic information that are typically not used. In this way, the accuracy of the diagnosis of a disease condition of a subject can be improved. In some embodiments, these copy number values are in the form of dimension reduction components. In some embodiments, these copy number values are not in the form of dimension reduction components.

Aspects of the present disclosure address the issue of missed or incorrect cancer diagnosis by using both on-target regions and off-target regions to more robustly diagnose cancer in patients. The use of the expanded set of regions—both on-target and off-target regions—to train a classifier can result in an improved accuracy of the detection. The data from on-target and off-target regions used for training the classifier can be obtained by applying mathematical transformation functions on the acquired sequencing data. Examples of such mathematical transformations include normalization (e.g., normalization for guanine-cytosine (GC) content) and dimensionality reduction (e.g., principal component analysis (PCA)) correction. The mathematical transformations can conserve computational resources by reducing the errors and/or sparsity of the sequencing data. The classifier can be trained using this expanded set of regions using a machine learning algorithm such as a neural network algorithm (e.g., a convolutional neural network), a support vector machine algorithm, a Naïve Bayes algorithm, a nearest neighbor algorithm, a boosted trees algorithm, a random forest algorithm, a decision tree algorithm, a multi-category logistic regression algorithm, a linear model, or a linear regression algorithm.

One aspect of the present disclosure provides a method of determining whether a subject of a species has a disease condition in a set of disease conditions. The method comprises, at a computer system comprising at least one processor and a memory storing at least one program for execution by the at least one processor, the at least one program comprising instructions for obtaining a test dataset, in electronic form, that comprises a first plurality of bin values, each respective bin value in the first plurality of bin values for a corresponding bin in a first plurality of bins. Each respective bin in the first plurality of bins represents a corresponding region of a reference genome of the species. The first plurality of bins collectively represents a first portion of the reference genome. In some embodiments, the first plurality of bins comprises one hundred bins. The first plurality of bin values are derived from a targeted sequencing of a plurality of nucleic acids from a biological sample of the subject. The plurality of nucleic acids are enriched using a plurality of probes before the targeted sequencing. Each probe in the plurality of probes includes a nucleic acid sequence that corresponds to one or more bins in the first plurality of bins.

In some embodiments, the at least one program comprises instructions for determining a plurality of copy number values at least in part from the first plurality of bin values.

In some embodiments, the at least one program comprises instructions for inputting at least the plurality of copy number values into a trained classifier, thereby determining whether the subject has a disease condition in the set of disease conditions.

In some embodiments, the test dataset further comprises a second plurality of bin values and the second plurality of bin values is also derived from the targeted sequencing of the plurality of nucleic acids from the biological sample of the subject. In such embodiments, each respective bin value in the second plurality of bin values is for a corresponding bin in a second plurality of bins. In some embodiments, each respective bin in the second plurality of bins represents a corresponding region of the reference genome, and the second plurality of bins collectively represents a second portion of the reference genome that does not overlap with the first portion. In some embodiments the second portion of the reference genome comprises 0.5 megabases of the reference genome. Further, in such embodiments the instruction for determining the plurality of copy number values further comprises determining the plurality of copy number values at least in part from the second plurality of bin values.

In some embodiments, the set of disease conditions is a set of cancer conditions and the determined disease condition is a cancer condition.

In some embodiments, the determined cancer condition is adrenal cancer, biliary track cancer, bladder cancer, bone/bone marrow cancer, brain cancer, cervical cancer, colorectal cancer, cancer of the esophagus, gastric cancer, head/neck cancer, hepatobiliary cancer, kidney cancer, liver cancer, lung cancer, ovarian cancer, pancreatic cancer, pelvis cancer, pleura cancer, prostate cancer, renal cancer, skin cancer, stomach cancer, testis cancer, thymus cancer, thyroid cancer, uterine cancer, lymphoma, melanoma, multiple myeloma, leukemia, or a combination thereof.

In some embodiments, the determined cancer condition is a predetermined stage of adrenal cancer, biliary track cancer, bladder cancer, bone/bone marrow cancer, brain cancer, cervical cancer, colorectal cancer, cancer of the esophagus, gastric cancer, head/neck cancer, hepatobiliary cancer, kidney cancer, liver cancer, lung cancer, ovarian cancer, pancreatic cancer, pelvis cancer, pleura cancer, prostate cancer, renal cancer, skin cancer, stomach cancer, testis cancer, thymus cancer, thyroid cancer, uterine cancer, lymphoma, melanoma, multiple myeloma, or leukemia.

In some embodiments, the plurality of nucleic acids are cell-free nucleic acids from the biological sample. In some embodiments, the plurality of nucleic acids are DNA or RNA.

In some embodiments, the targeted sequencing is targeted DNA methylation sequencing. For example, in some embodiments, the targeted DNA methylation sequencing detects one or more 5-methylcytosine (5mC) and/or 5-hydroxymethylcytosine (5hmC) in the plurality of nucleic acids. In some instances, the targeted DNA methylation sequencing comprises conversion of one or more unmethylated cytosines or one or more methylated cytosines, in the plurality of nucleic acids, to a corresponding one or more uracils. In some embodiments, the targeted DNA methylation sequencing comprises conversion of one or more unmethylated cytosines, in the plurality of nucleic acids, to a corresponding one or more uracils, and the DNA methylation sequence reads out the one or more uracils as one or more corresponding thymines. In some embodiments, the targeted DNA methylation sequencing comprises conversion of one or more methylated cytosines, in the plurality of nucleic acids, to a corresponding one or more uracils, and the DNA methylation sequence reads out the one or more 5mC or 5hmC as one or more corresponding thymines. In some embodiments, the conversion of one or more unmethylated cytosines or one or more methylated cytosines comprises a chemical conversion, an enzymatic conversion, or combinations thereof.

In some embodiments, each respective bin value in the first plurality of bin values is representative of a respective number of unique cell-free nucleic acid fragments in the biological sample that align to the portion of the reference genome represented by the bin corresponding to the respective bin value as determined by the targeted sequencing. In some embodiments, each cell-free nucleic acid fragment in the respective number of unique cell-free nucleic acid fragments is represented by one or more sequence reads from the targeted sequencing that contribute to the respective bin value.

In some embodiments, each respective bin value in the first plurality of bin values is representative of an average length of the unique cell-free nucleic acid fragments in the biological sample that align to the portion of the reference genome represented by the bin corresponding to the respective bin value as determined by the targeted sequencing.

In some embodiments, each respective bin value in the first plurality of bin values is representative of a number of unique cell-free nucleic acid fragments in the biological sample that have at least one terminal position within the portion of the reference genome represented by the bin corresponding to the respective bin value as determined by the targeted sequencing.

In some embodiments, each respective bin value in the first plurality of bin values and the second plurality of bins values is representative of a respective number of unique cell-free nucleic acid fragments in the biological sample that align to the portion of the reference genome represented by the bin corresponding to the respective bin value. In some embodiments, each cell-free nucleic acid fragment in the respective number of unique cell-free nucleic acid fragments is represented by one or more sequence reads contributing to the respective bin value.

In some embodiments, each respective bin value in the first plurality of bin values is representative of a number of unique cell-free nucleic acid fragments in the biological sample that both (i) align to the first portion of the reference genome corresponding to the respective bin and (ii) have a predetermined methylation pattern. In some embodiments, each cell-free nucleic acid fragment in the number of unique cell-free nucleic acid fragments is represented by one or more sequence reads from the targeted sequencing.

In some embodiments, each respective bin value in the first plurality of bin values or the second plurality of bin values is representative of a number of unique cell-free nucleic acid fragments in the biological sample that both (i) align to the portion of the reference genome corresponding to the bin corresponding to the respective bin value and (ii) have a predetermined methylation pattern, and each cell-free nucleic acid fragment in the number of unique cell-free nucleic acid fragments is represented by one or more sequence reads from the targeted sequencing with the plurality of probes that contribute to the respective bin value.

In some embodiments, the determining whether the subject has a disease condition in a set of disease conditions deems the subject to have a particular disease condition in the set of disease conditions.

In some embodiments, the subject is deemed to have the particular disease condition in the set of disease conditions when the trained classifier predicts the particular disease condition with a higher probability than all other disease conditions in the set of disease conditions.

In some embodiments, the set of disease conditions comprises two disease conditions. In some embodiments, the set of disease conditions includes a first disease condition that is absence of disease.

In some embodiments, the determining further comprises extracting a plurality of features from the first plurality of bin values using a feature extraction method and the inputting further comprises applying the plurality of features, in addition to the plurality of copy number values, to the trained classifier to determine whether the subject has the disease condition in the set of disease conditions.

In some embodiments, the method further comprises normalizing each respective bin value in the first plurality of bin values. In some embodiments, the method further comprises normalizing each respective bin value in the first plurality of bin values and each respective bin value in the second plurality of bin values.

In some embodiments, the normalizing, at least in part, comprises determining a first measure of central tendency across the first plurality of bin values, and replacing each respective bin value in the first plurality of bin values with the respective bin value divided by the first measure of central tendency. In some embodiments, the normalizing, at least in part, comprises determining a first measure of central tendency across the first and second plurality of bin values, an replacing each respective bin value in the first and second plurality of bin values with the respective bin value divided by the first measure of central tendency. In some embodiments, the first measure of central tendency is an arithmetic mean, weighted mean, midrange, midhinge, trimean, Winsorized mean, mean, or mode across the first plurality of bin values.

In some embodiments, the normalizing, at least in part, comprises, for each respective bin value bvᵢ in the first plurality of bin values, replacing the respective bin value with

$$b {v}_{i}^{*} ,$$

where:

$$b {v}_{i}^{*} = log ( \frac{{bv}_{i}}{measure of central tendency ( {bv}_{ik} )} )$$

and where measure of central tendency (bvᵢₖ) is a respective second measure of central tendency of bin value

$$b {v}_{i}^{*}$$

for respective bin i across a plurality of reference healthy subjects. In some embodiments, each bvᵢₖ for respective subject k in the plurality of reference healthy subjects is obtained by targeted panel sequencing cell-free nucleic acids in a biological sample from respective healthy subject k with the plurality of probes.

In some embodiments, the normalizing, at least in part, comprises for each respective bin value bvᵢ in the first and second plurality of bin values, replacing the respective bin value with

$$b {v}_{i}^{*} ,$$

where:

$$b {v}_{i}^{*} = log ( \frac{{bv}_{i}}{measure of central tendency ( {bv}_{ik} )} )$$

and where measure of central tendency (bvᵢₖ) is a respective second measure of central tendency of bin value

$$b {v}_{i}^{*}$$

for respective bin i across a plurality of reference healthy subjects. In some embodiments, each bvᵢₖ for respective subject k in the plurality of reference healthy subjects is obtained by targeted panel sequencing of a biological sample from respective healthy subject k where the nucleic acids from the biological sample from the respective healthy subject k have been enriched using a plurality of probes before sequencing analysis.

In some embodiments, the respective second measure of central tendency is an arithmetic mean, weighted mean, midrange, midhinge, trimean, Winsorized mean, mean, or mode of bin value

$$b {v}_{i}^{*}$$

for respective bin i across the plurality of reference healthy subjects.

In some embodiments, the normalizing, at least in part, comprises replacing each respective bin value in the first plurality of bin values with the respective bin value corrected for a respective first GC bias in the first plurality of bin values.

In some embodiments, the respective first GC bias is defined by a first equation for a curve or line fitted to a first plurality of two-dimensional points, wherein each respective two-dimensional point in the first plurality of two-dimensional points includes (i) a first value that is the respective GC content of the corresponding portion of the reference genome of the species represented by the respective bin in the first plurality of bins corresponding to the respective two-dimensional point and (ii) a second value that is the bin value in the first plurality of bin values for the respective bin, and the replacing each respective bin value in the first plurality of bin values with the respective bin value corrected for a respective first GC bias in the first plurality of bin values comprises subtracting a predicted GC bias for the respective bin, derived by inputting the proportion of G and C bases of the corresponding portion of the reference genome represented by the respective bin into the first equation, from the respective bin value.

In some embodiments, the normalizing comprises replacing each respective bin value in the first plurality of bin values with the respective bin value corrected for a respective first GC bias in the first plurality of bin values, and replacing each respective bin value in the second plurality of bin values with the respective bin value corrected for a respective second GC bias in the second plurality of bin values.

In some embodiments, the respective first GC bias is defined by a first equation for a curve or line fitted to a first plurality of two-dimensional points, where each respective two-dimensional point in the first plurality of two-dimensional points includes (i) a first value that is the respective GC content of the corresponding portion of the reference genome of the species represented by the respective bin in the first plurality of bins corresponding to the respective two-dimensional point and (ii) a second value that is the bin value in the first plurality of bin values for the respective bin. In some embodiments, the replacing each respective bin value in the first plurality of bin values with the respective bin value corrected for a respective first GC bias in the first plurality of bin values comprises subtracting a predicted GC bias for the respective bin from the respective bin value, where the predicted GC bias for the respective bin is derived by inputting the proportion of G and C bases of the corresponding portion of the reference genome represented by the respective bin into the first equation. In some embodiments, the respective second GC bias is defined by a second equation for a curve or line fitted to a second plurality of two-dimensional points, where each respective two-dimensional point in the second plurality of two-dimensional points includes (i) a third value that is the respective GC content of the corresponding portion of the reference genome of the species represented by the respective bin in the second plurality of bins corresponding to the respective two-dimensional point and (ii) a fourth value that is the bin value in the second plurality of bin values for the respective bin, and the replacing each respective bin value in the second plurality of bin values with the respective bin value corrected for a respective second GC bias in the second plurality of bin values comprises subtracting a predicted GC bias for the respective bin from the respective bin value, where the predicted GC bias for the respective bin is derived by inputting the proportion of G and C bases of the corresponding portion of the reference genome represented by the respective bin into the second equation.

In some embodiments the normalizing, at least in part, comprises, for each respective bin value

$$b {v}_{i}^{* *}$$

in the first plurality of bin values, replacing the respective bin value with

$$b {v}_{i}^{* * *} ,$$

where:

$$b {v}_{i}^{* * *} = b {v}_{i}^{* *} - \hat{b} {v}_{i}^{* *}$$

and where

$$\hat{b} {v}_{i}^{* *}$$

represents a linear model of PC₁, . . . , PC_{N}, N is a positive integer between 2 and 50, and PC₁, . . . , PC_{N} are a top number of dimension reduction components in a first plurality of dimension reduction components derived from subjecting respective normalized bin values for the first plurality of bins, obtained from targeted sequencing of each respective biological sample from each respective healthy subject in a plurality of reference healthy subjects, where the nucleic acids from the respective biological sample have been enriched using the plurality of probes before sequencing analysis, to a first unsupervised dimension reduction algorithm.

In some embodiments, the normalizing, at least in part, comprises, for each respective bin value

$$b {v}_{i}^{* *}$$

in the first and second plurality of bin values, replacing the respective bin value with

$$b {v}_{i}^{* * *} ,$$

where:

$$b {v}_{i}^{* * *} = b {v}_{i}^{* *} - \hat{b} {v}_{i}^{* *}$$

and where

$$\hat{b} {v}_{i}^{* *}$$

represents a linear model of PC₁, . . . , PC_{N}, N is a positive integer between 2 and 50, and PC₁, . . . , PC_{N} are a top number of dimension reduction components in a first plurality of dimension reduction components derived from subjecting respective normalized bin values for the first plurality of bins and the second plurality of bins, obtained from targeted sequencing of each respective biological sample from each respective healthy subject in the plurality of reference healthy subjects, where the nucleic acids from the respective biological sample have been enriched using the plurality of probes before sequencing analysis, to a first unsupervised dimension reduction algorithm.

In some embodiments, the first unsupervised dimension reduction algorithm is a principal component analysis algorithm, a random projection algorithm, an independent component analysis algorithm, or a feature selection method.

In some embodiments, N is between three and ten.

In some embodiments, the determining further comprises filtering the first plurality of bin values and the second plurality of bin values by removing at least one bin value associated with at least one of a germline mutation, high variability, or low mappability.

In some embodiments, each corresponding region of the reference genome for a respective bin in the first plurality of bins is associated with one or more probes in the plurality of probes.

In some embodiments, each region of the reference genome that corresponds to a respective bin in the second plurality of bins is different from each region of the reference genome that corresponds to a respective bin in the first plurality of bins.

In some embodiments, each region of the reference genome that corresponds to a respective bin in the second plurality of bins comprises an off-target region. In some such embodiments, the corresponding region of each respective bin in the first plurality of bins is an on-target region in a plurality of on-target regions, and the off-target region is defined as a region of the reference genome that does not overlap with an on-target region in the plurality of on-target regions.

In some embodiments, the first portion of the reference genome collectively encompasses between 0.5 megabase and 50 megabases of unique sequences in the reference genome, and the plurality of probes consists of between 250 and 2,000,000 probes.

In some embodiments, a probe in the plurality of probes is designed to bind and enrich nucleic acids in the biological sample that contain at least one predetermined CpG site.

In some embodiments, each probe in the plurality of probes is designed to bind and enrich nucleic acids in the biological sample that contain at least one predetermined CpG site.

In some embodiments, a probe in the plurality of probes is designed to bind and enrich nucleic acids in the biological sample that contain 50 or fewer predetermined CpG sites, 40 or fewer predetermined CpG sites, 30 or fewer predetermined CpG sites, 25 or fewer predetermined CpG sites, 22 or fewer predetermined CpG sites, 20 or fewer predetermined CpG sites, 18 or fewer predetermined CpG sites, 15 or fewer predetermined CpG sites, 12 or fewer predetermined CpG sites, 10 or fewer predetermined CpG sites, 5 or fewer predetermined CpG sites, or 3 or fewer predetermined CpG sites.

In some embodiments, each probe in the plurality of probes is designed to bind and enrich nucleic acids in the biological sample that contain 50 or fewer predetermined CpG sites, 40 or fewer predetermined CpG sites, 30 or fewer predetermined CpG sites, 25 or fewer predetermined CpG sites, 22 or fewer predetermined CpG sites, 20 or fewer predetermined CpG sites, 18 or fewer predetermined CpG sites, 15 or fewer predetermined CpG sites, 12 or fewer predetermined CpG sites, 10 or fewer predetermined CpG sites, 5 or fewer predetermined CpG sites, or 3 or fewer predetermined CpG sites.

In some embodiments, each bin in the first plurality of bins does not overlap with another bin in the first plurality of bins.

In some embodiments, each bin in the first plurality of bins has a size selected from the group consisting of between about 10 and about 1,000 nucleotides (nt), between about 50 and about 500 nt, and between about 100 and about 250 nt.

In some embodiments, each bin in the second plurality of bins has a size between about 10,000 base pairs and about 250,000 base pairs.

In some embodiments, each bin in the second plurality of bins has a size selected from the group consisting of between about 10,000 and about 500,000 nt, between about 50,000 and about 250,000 nt, and between about 100,000 and about 150,000 nt.

In some embodiments, each bin in the second plurality of bins has the same length.

In some embodiments, each bin in the first plurality of bins has a first length, each bin in the first plurality of bins has a second length, the first length is other than the second length, the first length is between about 100 base pairs and about 250,000 base pairs, and the second length is between about 10,000 base pairs and about 250,000 base pairs.

In some embodiments, each bin in the first plurality of bins and the second plurality of bins has the same or different length.

In some embodiments, each bin in the first plurality of bins is flanked by a respective pair of buffer regions, and each respective pair of buffer regions is excluded from the second portion of the reference genome collectively represented by the second plurality of bins.

In some embodiments, each buffer region in a respective pair of buffer regions has a length from about 100 base pairs to about 1000 base pairs.

In some embodiments, each buffer region in a respective pair of buffer regions has a length of about 200 base pairs.

In some embodiments, the first plurality of bin values and the second plurality of bin values are generated from counts of sequence reads from the targeted sequencing with the plurality of probes.

In some embodiments, the trained classifier is a neural network algorithm a support vector machine algorithm (SVM), a Naïve Bayes algorithm, a nearest neighbor algorithm, a random forest algorithm, a decision tree algorithm, a boosted trees algorithm, a regression algorithm, a logistic regression algorithm, a multi-category logistic regression algorithm, a linear discriminant analysis algorithm, or a clustering algorithm.

In some embodiments, the trained classifier is trained using on-target bin values and off-targets bin values obtained from targeted panel sequencing of a plurality of samples, using the plurality of probes.

In some embodiments, the biological sample is a blood sample.

In some embodiments, the biological sample comprises blood, whole blood, plasma, serum, urine, cerebrospinal fluid, fecal, saliva, sweat, tears, pleural fluid, pericardial fluid, or peritoneal fluid of the subject.

In some embodiments, the disease condition is clonal hematopoiesis.

In some embodiments, the biological sample comprises blood, whole blood, plasma, serum, urine, cerebrospinal fluid, fecal, saliva, sweat, tears, pleural fluid, pericardial fluid, or peritoneal fluid of the subject.

In some embodiments, the determining the plurality of copy number values comprises calculating the plurality of copy number values as a second plurality of dimension reduction values, each respective dimension reduction value in the second plurality of dimension reduction values is calculated using a corresponding weighted combination of all or a portion of the first plurality of bin values that is specified by a corresponding dimension reduction component in a second plurality of dimension reduction components, and the second plurality of dimension reduction components is obtained from subjecting sequence reads, obtained by targeted sequencing of cell-free nucleic acids in each biological sample from each respective healthy subject in a plurality of reference healthy subjects using the plurality of probes, to a second unsupervised dimension reduction algorithm.

In some embodiments, the determining the plurality of copy number values comprises calculating the plurality of copy number values as a second plurality of dimension reduction values, each respective dimension reduction value in the second plurality of dimension reduction values is calculated using a corresponding weighted combination of all or a portion of the first and second plurality of bin values that is specified by a corresponding dimension reduction component in a second plurality of dimension reduction components, and the second plurality of dimension reduction components is obtained by subjecting a corresponding first plurality and corresponding second plurality of reference bin values obtained by targeted sequencing of cell-free nucleic acids in a corresponding biological sample of the respective healthy subject using the plurality of probes, for each reference healthy subject in a plurality of reference healthy subjects, to a second unsupervised dimension reduction algorithm.

In some embodiments, the second dimension reduction algorithm is a principal component analysis algorithm, a random projection algorithm, an independent component analysis algorithm, or a feature selection method.

In some embodiments, the second unsupervised dimension reduction algorithm is the feature selection method, and the feature selection method is a sequential backward selection algorithm.

In some embodiments, the second unsupervised dimension reduction algorithm is a principal component analysis algorithm, and the second plurality of dimension reduction components is between five and five hundred dimension reduction components.

In some embodiments, the method further comprises applying a treatment regimen to the subject based at least in part the disease condition identified by the classifier. In some such embodiments the disease condition is a cancer condition, and the treatment regimen comprises applying an agent for cancer to the subject. In some such embodiments, the agent for cancer is a hormone, an immune therapy, radiography, or a cancer drug. In some such embodiments, the agent for cancer is Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, or Bortezomib.

In some embodiments, the disease condition is a cancer condition, and the subject has been treated with an agent for cancer and the method further comprises evaluating a response of the subject to the agent for cancer using the disease condition determined by the classifier. In some such embodiments, the agent for cancer is a hormone, an immune therapy, radiography, or a cancer drug. In some such embodiments, the agent for cancer is Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, or Bortezomib.

In some embodiments, the disease condition is a cancer condition, and the subject has been treated with an agent for cancer and the method further comprises evaluating a response of the subject to the agent for cancer using the disease condition determined by the classifier.

In some embodiments, the disease condition is a cancer condition, and the subject has been subjected to a surgical intervention to address the cancer condition and the method further comprises evaluating a response of the subject to the agent for cancer using the disease condition determined by the classifier.

In another aspect, disclosed herein are methods and systems for obtaining a trained classifier for determining a disease condition in a set of disease conditions. In some embodiments, the trained classifier is obtained using sequencing data from a group of training subjects known to have a first disease condition in the set of disease conditions. In some embodiments, the trained classifier is obtained using sequencing data from a group of training subjects known to have a first disease condition in the set of disease conditions and another group of training subjects known to have a second disease condition in the set of disease conditions. In some embodiments, a disease condition includes the condition of not having a particular disease. In some embodiments, the trained classifier distinguishes between a cancer condition and a non-cancer condition. In some embodiments, the trained classifier distinguishes between a first cancer condition and a second cancer condition.

Another aspect of the present disclosure provides a non-transitory computer-readable storage medium having stored thereon program code instructions that, when executed by a processor, cause the processor to perform any of the methods of the present disclosure.

Another aspect of the present disclosure provides a computer system comprising one or more processors and a non-transitory computer-readable medium including computer-executable instructions that, when executed by the one or more processors, cause the processors to perform and of the methods provided in the present disclosure.

### INCORPORATION BY REFERENCE

All publications, patents, and patent applications herein are incorporated by reference in their entireties. In the event of a conflict between a term herein and a term in an incorporated reference, the term herein controls.

### BRIEF DESCRIPTION OF THE DRAWINGS

The file of this patent contains at least one at least one drawing/photograph executed in color. Copies of this patent with color drawing(s)/photograph(s) will be provided by the Office upon request and payment of the necessary fee.

The implementations disclosed herein are illustrated by way of example, and not by way of limitation, in the figures of the accompanying drawings. Like reference numerals refer to corresponding parts throughout the several views of the drawings.

FIG. 1 is a block diagram illustrating an example of a computing system in accordance with some embodiments of the present disclosure.

FIG. 2 is a schematic diagram of processing performed in accordance with some embodiments of the present disclosure.

FIG. 3 is a schematic diagram of a reference genome with bins for on-target and off-target regions, set up in accordance with some embodiments of the present disclosure.

FIGS. 4A, 4B, 4C, 4D, and 4E illustrate examples of flowcharts of a method of determining whether a subject of a species has a disease condition in a set of disease conditions, in accordance with some embodiments of the present disclosure, in which optional steps are designated by dashed boxes.

FIG. 5 illustrates an example flowchart of a method of training a classifier to determine whether a subject has a disease condition, in accordance with some embodiments of the present disclosure.

FIG. 6 shows graphs illustrating results of projecting data obtained from on-target regions (top panel) and off-target regions (bottom panel) from the ART sequencing (paired cell-free DNA and white blood cell targeted sequencing of 507 genes with 60,000× coverage, as described in Example 1 below) of the samples in the CCGA study (Example 1), by projecting the samples on top principal components (PC) from principal component analysis (PCA), the graphs illustrating a comparison of the ability to discern cancer (grey) from non-cancer (black), in accordance with some embodiments of the present disclosure.

FIGS. 7A and 7B illustrate an example of copy number segmentation plots of copy number analysis for on-target (FIG. 7A) and off-target regions (FIG. 7B) with the cfDNA sample from a known cancer patient (labeled as P006050), where log-transformed copy number signal values of the patient over controls (e.g., sample/mean (controls)) are clustered and plotted for each chromosome, in accordance with some embodiments of the present disclosure.

FIGS. 8A and 8B illustrate another example of copy number segmentation plots of copy number analysis for on-target (FIG. 8A) and off-target (FIG. 8B) regions with the cfDNA sample from a known cancer patient (labeled as P002WQ0), where log-transformed copy number signal values of the patient over controls (e.g., sample/mean (controls)) are clustered and plotted for each chromosome, in accordance with some embodiments of the present disclosure.

FIGS. 9A and 9B illustrate another example of copy number segmentation plots illustrating copy number analysis for on-target (FIG. 9A) regions and off-target (FIG. 9B) regions with the cfDNA sample from a known cancer patient (labeled as P004MQ1), where log-transformed copy number signal values of the patient over controls (e.g., sample/mean (controls)) are clustered and plotted for each chromosome, in accordance with some embodiments of the present disclosure.

FIGS. 10A and 10B illustrate an example of copy number segmentation plots illustrating copy number analysis for on-target (FIG. 10A) and off-target (FIG. 10B) regions with cfDNA sample from a known non-cancer subject (labeled as P0063E0), where log-transformed copy number signal values of the subject over controls (e.g., sample/mean (controls)) are clustered and plotted for each chromosome, in accordance with some embodiments of the present disclosure.

FIG. 11 illustrates variance in the data captured when different numbers of PCs are used, for on-target regions (top panel) and off-target regions (bottom panel), in accordance with some embodiments of the present disclosure.

FIG. 12 illustrates binary classification performance of a classifier that uses on-target regions (top panel) or off-target regions (bottom panel), and different number of PCs, for all analyzed cancers from the CCGA study, in accordance with some embodiments of the present disclosure.

FIG. 13 illustrates binary classification performance of a classifier that uses combined on-target and off-target regions, and different number of PCs, for all analyzed cancers from the CCGA study, in accordance with some embodiments of the present disclosure.

FIG. 14 illustrates binary classification performance of a classifier that uses on-target regions, off-target regions, or combined data including both on-target and off-target regions, for 100 PCs (top panel) and 50 PCs (bottom panel), for all analyzed cancers from the CCGA study, in accordance with some embodiments of the present disclosure.

FIG. 15 illustrates results of binary classification performance of a classifier that uses on-target regions, off-target regions, or combined data including both on-target and off-target regions, for 5, 20, 50, and 100 PCs (top panel) and 50 PCs (bottom panel) and for 95%, 98% and 99% specificities, for all analyzed cancers from the CCGA study, in accordance with some embodiments of the present disclosure.

FIGS. 16A, 16B, and 16C illustrate comparison of classification performance of a classifier trained using on-target regions and a classifier trained using off-target regions from all cancer samples from the CCGA study, with 95% specificity (FIG. 16A), 98% specificity (FIG. 16B), and 99% specificity (FIG. 16C).

FIG. 17 illustrates results of estimating a probability of cancer by cancer type for samples from the CCGA study, using on-target regions (top), off-target regions (middle), or combined data (bottom) including both on-target and off-target regions, in accordance with some embodiments of the present disclosure. Here, the classifier has been trained on all cancer samples represented in the CCGA study.

FIGS. 18A and 18B illustrate results of estimating a probability of cancer by cancer stage for samples from the CCGA study, using on-target regions (top left), off-target regions (top right), or combined data (bottom) including both on-target and off-target regions, in which results are shown for non-cancer, cancer stages I, II, III, and IV, and for non-informative estimates, in accordance with some embodiments of the present disclosure.

FIG. 19 illustrates binary classification performance of a classifier that uses on-target regions or off-target regions, and different number of PCs, for high signal cancers from the CCGA study, in accordance with some embodiments of the present disclosure.

FIG. 20 illustrates the binary classification performance of a classifier that uses combined data including both on-target and off-target regions, and different number of PCs, for high signal cancers from the CCGA study, in accordance with some embodiments of the present disclosure.

FIG. 21 are graphs illustrating binary classification performance of a classifier that uses on-target regions, off-target regions, or combined data including both on-target and off-target regions, for 100 PCs (left panel) and 50 PCs (right panel), for high signal cancers from the CCGA study, in accordance with some embodiments of the present disclosure.

FIG. 22 illustrates results of binary classification performance of a classifier that uses on-target regions, off-target regions, or combined data including both on-target and off-target regions, for 5, 20, 50, and 100 PCs (top panel) and 50 PCs (bottom panel) and for 95%, 98% and 99% specificities, for high-signal cancers from the CCGA study, in accordance with some embodiments of the present disclosure.

FIGS. 23A, 23B, 23C, and 23D illustrate comparison of classification performance of a classifier trained using on-target regions and a classifier trained using off-target regions from high-signal cancer samples from the CCGA study, with 95% specificity (FIG. 23B), 98% specificity (FIG. 23C), and 99% specificity (FIG. 23D), in accordance with some embodiments of the present disclosure.

FIG. 24 illustrates results of estimating a probability of cancer by cancer type for high signal cancer samples from the CCGA study, using on-target regions, off-target regions, or combined data including both on-target and off-target regions, in accordance with some embodiments of the present disclosure. Here, the classifier has been trained on non-cancer samples and on samples of high signal cancers present in the CCGA study.

FIGS. 25A, 25B, and 25C illustrate results of estimating a probability of cancer by cancer stage for high signal cancer samples from the CCGA study, using on-target regions (FIG. 25A), off-target regions (FIG. 25B), or combined data including both on-target and off-target regions (FIG. 25C), in which results are shown for non-cancer, cancer stages I, II, III, and IV, and for non-informative estimates, in accordance with some embodiments of the present disclosure.

FIG. 26 is a flowchart describing a process of sequencing nucleic acids, in accordance with an aspect of the present disclosure.

FIG. 27 is an illustration of a part of the process of sequencing nucleic acids to obtain methylation information and methylation state vectors, in accordance with an aspect of the present disclosure.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The present disclosure provides techniques for improved cancer diagnosis using a computer-implemented method that takes advantage of as much genomic information as possible. Precise and timely cancer diagnosis still remains an area for further improvements despite recent advances in sequencing technologies. Moreover, although modern sequencing generates large amounts of data based on patient's tissue and liquid samples, identifying cancer signatures in the data remains nontrivial, even with advanced computational approaches.

Furthermore, in targeted panel sequencing, which allows analysis of genomic regions of interest using specific probes, the regions of interest (corresponding to the probes) are used for analysis and subsequent decision-making. Sequencing data acquired from other regions, other than regions of interest, as a result of “accidental” or unintentional sequencing, is typically discarded from further consideration. In this way, laboratory and computer resources expended to acquire the sequencing data using the targeted panel sequencing, are essentially wasted. The waste includes the burden on the equipment, use of various reagents, and, notably, use of computer hardware resources.

Accordingly, the implementations described herein provide various technical solutions that can make use of both on-target regions (corresponding to probes in a targeted panel sequencing) and off-target regions that are the result of accidental sequencing and are thus typically discarded. In this way, the present disclosure can allow improved utilization of computer resources, thereby improving computer technology. The present techniques can include training a classifier to discriminate between cancer conditions in a cancer condition set, and for applying the trained classifier to determine a disease condition for a test subject of unknown status.

### Definitions

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value can be assumed. The term “about” can have the meaning as commonly understood by one of ordinary skill in the art. The term “about” can refer to ±10%. The term “about” can refer to ±5%.

As used herein, the term “biological sample,” “patient sample,” or “sample” refers to any sample taken from a subject, which can reflect a biological state associated with the subject, and that includes cell free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, the term “cancer” or “tumor” refers to an abnormal mass of tissue in which the growth of the mass surpasses and is not coordinated with the growth of normal tissue. A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites.

As used herein, the term “cancer condition” refers to breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, and gastric cancer. The term “cancer condition” also refers to a “non-cancer” condition of not having cancer or noncancerous condition. A cancer condition can be a predetermined stage of a breast cancer, a predetermined stage of a lung cancer, a predetermined stage of a prostate cancer, a predetermined stage of a colorectal cancer, a predetermined stage of a renal cancer, a predetermined stage of a uterine cancer, a predetermined stage of a pancreatic cancer, a predetermined stage of a cancer of the esophagus, a predetermined stage of a lymphoma, a predetermined stage of a head/neck cancer, a predetermined stage of a ovarian cancer, a predetermined stage of a hepatobiliary cancer, a predetermined stage of a melanoma, a predetermined stage of a cervical cancer, a predetermined stage of a multiple myeloma, a predetermined stage of a leukemia, a predetermined stage of a thyroid cancer, a predetermined stage of a bladder cancer, or a predetermined stage of a gastric cancer. A cancer condition can also be a survival metric, which can be a predetermined likelihood of survival for a predetermined period of time. For example, the survival metric can be defined as the difference in time (e.g., years or months) between the date of the initial diagnosis of a disease or condition (e.g., cancer) until the date of expiry of the patient due to that disease or condition.

As used herein, the term “Circulating Cell-free Genome Atlas” or “CCGA” is defined as an observational clinical study that prospectively collects blood and tissue from newly diagnosed cancer patients as well as blood from subjects who do not have a cancer diagnosis. The purpose of the study is to develop a pan-cancer classifier that distinguishes cancer from non-cancer and identifies tissue of origin. Example 1 provides further details of the CCGA study.

The term “classification” can refer to any number(s) or other characters(s) that are associated with a particular property of a sample. For example, a “+” symbol (or the word “positive”) can signify that a sample is classified as having deletions or amplifications. In another example, the term “classification” can refer to an amount of tumor tissue in the subject and/or sample, a size of the tumor in the subject and/or sample, a stage of the tumor in the subject, a tumor load in the subject and/or sample, and presence of tumor metastasis in the subject. The classification can be binary (e.g., positive or negative) or have more levels of classification (e.g., fall into some numeric range supported or outputted by the classifier). The terms “cutoff” and “threshold” can refer to predetermined numbers used in an operation. For example, a cutoff size can refer to a size above which fragments are excluded. A threshold value can be a value above or below which a particular classification applies. Either of these terms can be used in either of these contexts.

As used herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), and/or DNA analogs (e.g., containing base analogs, sugar analogs and/or a non-native backbone and the like), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. A nucleic acid may be prepared using a nucleic acid obtained from a subject as a template.

As used herein, the term “cell-free nucleic acids” refers to nucleic acid molecules that can be found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject. Cell-free nucleic acids originate from one or more healthy cells and/or from one or more cancer cells Cell-free nucleic acids are used interchangeably as circulating nucleic acids. Examples of the cell-free nucleic acids include but are not limited to RNA, mitochondrial DNA, or genomic DNA. As used herein, the terms “cell free nucleic acid,” “cell free DNA,” and “cfDNA” are used interchangeably.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map sequence reads obtained from sequencing a sample from the subject. A reference genome can refer to a haploid or diploid genome to which sequence reads from the biological sample can be aligned and compared. An example of constitutional sample can be DNA of white blood cells obtained from the subject. For a haploid genome, there can be one nucleotide at each locus. For a diploid genome, heterozygous loci can be identified; each heterozygous locus can have two alleles, where either allele can allow a match for alignment to the locus.

As used herein, the term “CpG site” refers to a region of a DNA molecule where a cytosine nucleotide is followed by a guanine nucleotide in the linear sequence of bases along its 5′ to 3′ direction. “CpG” is a shorthand for 5′-C-phosphate-G-3′ that is cytosine and guanine separated by one phosphate group; phosphate links any two nucleotides together in DNA. Cytosines in CpG dinucleotides can be methylated to form 5-methylcytosine.

As used herein, the term “hypomethylated” or “hypermethylated” refers to a methylation status of a DNA molecule containing multiple CpG sites (e.g., more than 3, 4, 5, 6, 7, 8, 9, 10, etc.) where a high percentage of the CpG sites (e.g., more than 80%, 85%, 90%, or 95%, or any other percentage within the range of 50%-100%) are unmethylated or methylated, respectively.

As used herein, the phrase “healthy,” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used here, the term “high-signal cancer” means cancers with greater than 50% 5-year cancer-specific mortality. Examples of high-signal cancer include anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient. In some embodiments, “high signal cancers” refer to cancers that do not fall within the group of low signal cancers (e.g., uterine cancer, thyroid cancer, prostate cancer, and hormone-receptor-positive stage I/II breast cancer).

As used herein, the term “stage of cancer” (where the term “cancer” is either cancer generally or an enumerated cancer type) refers to whether cancer (or the enumerated cancer type when indicated) exists (e.g., presence or absence), a level of a cancer, a size of tumor, presence or absence of metastasis, the total tumor burden of the body, and/or other measure of a severity of a cancer (e.g., recurrence of cancer). The stage of cancer can be a number or other indicia, such as symbols, alphabet letters, and colors. The stage can be zero. The stage of cancer can also include premalignant or precancerous conditions (states) associated with mutations or a number of mutations. The stage of cancer can be used in various ways. For example, screening can check if cancer is present in someone who is not known previously to have cancer. Assessment can investigate someone who has been diagnosed with cancer to monitor the progress of cancer over time, study the effectiveness of therapies or to determine the prognosis. In some embodiments, the prognosis can be expressed as the chance of a subject dying of cancer, or the chance of the cancer progressing after a specific duration or time, or the chance of cancer metastasizing. Detection can comprise ‘screening’ or can comprise checking if someone, with suggestive features of cancer (e.g., symptoms or other positive tests), has cancer. A “level of pathology” can refer to level of pathology associated with a pathogen, where the level can be as described above for cancer. When the cancer is associated with a pathogen, a level of cancer can be a type of a level of pathology.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the terms “sequencing,” “sequence determination,” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein the term “sequencing breadth” refers to what fraction of a particular reference genome (e.g., human reference genome) or part of the genome has been analyzed. The denominator of the fraction can be a repeat-masked genome, and thus 100% can correspond to all of the reference genome minus the masked parts. A repeat-masked genome can refer to a genome in which sequence repeats are masked (e.g., sequence reads align to unmasked portions of the genome). Any parts of a genome can be masked, and thus one can focus on any particular part of a reference genome. Broad sequencing can refer to sequencing and analyzing at least 0.1% of the genome.

As used herein, the term “sequencing depth,” is interchangeably used with the term “coverage” and refers to the number of times a genomic location is surveyed during a sequencing process. For example, it can be reflected by the number of times that a locus is covered by a consensus sequence read corresponding to a unique nucleic acid target molecule aligned to the locus; e.g., the sequencing depth is equal to the number of unique nucleic acid target molecules covering the locus. The genomic location can be as small as a nucleotide, or as large as a chromosome arm, or as large as an entire genome. Sequencing depth can be expressed as “Yx”, e.g., 50×, 100×, etc., where “Y” refers to the number of times a genomic location is covered with a sequence corresponding to a nucleic acid target; e.g., the number of times independent sequence information is obtained covering the particular genomic location. In some embodiments, the sequencing depth corresponds to the number of genomes that have been sequenced. Sequencing depth can also be applied to multiple loci, or the whole genome, in which case Y can refer to the mean or average number of times a loci or a haploid genome, or a whole genome, respectively, is independently sequenced. When a mean depth is quoted, the actual depth for different loci included in the dataset can span over a range of values. In some embodiments, deep sequencing can refer to at least 100× in sequencing depth at a locus. In some embodiments, a sequencing depth of 10,000× or higher can be adopted in order to identify rare mutations.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, the term “true positive” (TP) refers to a subject having a condition. “True positive” can refer to a subject that has a tumor, a cancer, a precancerous condition (e.g., a precancerous lesion), a localized or a metastasized cancer, or a non-malignant disease. “True positive” can refer to a subject having a condition, and is identified as having the condition by an assay or method of the present disclosure.

As used herein, the term “true negative” (TN) refers to a subject that does not have a condition or does not have a detectable condition. True negative can refer to a subject that does not have a disease or a detectable disease, such as a tumor, a cancer, a precancerous condition (e.g., a precancerous lesion), a localized or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “single nucleotide variant” or “SNV” refers to a substitution of one nucleotide at a position (e.g., site) of a nucleotide sequence, e.g., a sequence corresponding to a target nucleic acid molecule from an individual, to a nucleotide that is different from the nucleotide at the corresponding position in a reference genome. A substitution from a first nucleobase X to a second nucleobase Y may be denoted as “X>Y.” For example, a cytosine to thymine SNV may be denoted as “C>T.” In some embodiments, an SNV does not result in a change in amino acid expression (a synonymous variant). In some embodiments, an SNV results in a change in amino acid expression (a non-synonymous variant).

As used herein, the terms “size profile” and “size distribution” can relate to the sizes of DNA fragments in a biological sample. A size profile can be a histogram that provides a distribution of an amount of DNA fragments at a variety of sizes. Various statistical parameters (also referred to as size parameters or just parameter) can distinguish one size profile to another. One parameter can be the percentage of DNA fragment of a particular size or range of sizes relative to all DNA fragments or relative to DNA fragments of another size or range.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a women or a child).

As used herein, the term “tissue” can correspond to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites.” In other instances, methylation may occur at a cytosine not part of a CpG site or at another nucleotide that is not cytosine; however, these are rarer occurrences. In this present disclosure, methylation is discussed in reference to CpG sites for the sake of clarity. Anomalous cfDNA methylation can identified as hypermethylation or hypomethylation, both of which may be indicative of cancer status. As is well known in the art, DNA methylation anomalies (compared to healthy controls) can cause different effects, which may contribute to cancer.

As used herein the term “methylation index” for each genomic site (e.g., a CpG site, a region of DNA where a cytosine nucleotide is followed by a guanine nucleotide in the linear sequence of bases along its 5′ to 3′ direction) can refer to the proportion of sequence reads showing methylation at the site over the total number of reads covering that site. The “methylation density” of a region can be the number of reads at sites within a region showing methylation divided by the total number of reads covering the sites in the region. The sites can have specific characteristics, (e.g., the sites can be CpG sites). The “CpG methylation density” of a region can be the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by sequence reads mapped to the 100-kb region. In some embodiments, this analysis is performed for other bin sizes, e.g., 50-kb or 1-Mb, etc. In some embodiments, a region is an entire genome or a chromosome or part of a chromosome (e.g., a chromosomal arm). A methylation index of a CpG site can be the same as the methylation density for a region when the region includes that CpG site. The “proportion of methylated cytosines” can refer the number of cytosine sites, “C's,” that are shown to be methylated (for example unconverted after bisulfite conversion) over the total number of analyzed cytosine residues, e.g., including cytosines outside of the CpG context, in the region. The methylation index, methylation density, and proportion of methylated cytosines are examples of “methylation levels.” One of skill in the art would understand that these parameters are devised to assess the extent or level of methylation in a particular sample and accordingly can be broadly defined so long as such definitions enable the assessment of an extent or a level of methylation in a sample. Additionally, such assessment can be performed for different genomic regions (e.g., from individual CpG sites, to nucleic acid fragments, to an entire gene and beyond); for example, a methylation index can sometimes simply refer to the number of methylated genes per sample. See Marzese et al. 2012 J Mol Diagnos 14(6), 613-622.

As used herein, the term “methylation profile” (also called methylation status) can include information related to DNA methylation for a region. Information related to DNA methylation can include a methylation index of a CpG site, a methylation density of CpG sites in a region, a distribution of CpG sites over a contiguous region, a pattern or level of methylation for each individual CpG site within a region that contains more than one CpG site, and non-CpG methylation. A methylation profile of a substantial part of the genome can be considered equivalent to the methylome. “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts (e.g., 5′-CHG-3′ and 5′-CHH-3′) where His adenine, cytosine, or thymine. Cytosine methylation can also be in the form of 5-hydroxymethylcytosine. Methylation of DNA can include methylation of non-cytosine nucleotides, such as N6-methyladenine. For example, methylation data (e.g., density, distribution, pattern, or level of methylation) from different genomic regions can be converted to one or more vector set and analyzed by methods and systems disclosed herein.

As used herein, the term “methylation state vector” or “methylation status vector” refers to a vector comprising multiple elements, where each element indicates methylation status of a methylation site in a DNA molecule comprising multiple methylation sites, in the order they appear from 5′ to 3′ in the DNA molecule. For example, <Mx, Mx+J, Mx+2>, <Mx, Mx+1, Ux+2>, . . . , <Ux, Ux+1, Ux+2> can be methylation vectors for DNA molecules comprising three methylation sites, where M represents a methylation site that is in a methylated state and U represents a methylation site in an unmethylated state. U.S. Patent Application No. 62/948,129, entitled “Cancer Classification Using Patch Convolutional Neural Networks,” filed Dec. 13, 2019, which is hereby incorporated by reference in its entirety, further discloses methods of determining methylation state vectors. For example, for each sequence read in a plurality of sequence reads obtained from a biological sample of a subject, a respective location and respective methylation state is determined for each of one or more CpG cites based on alignment to a reference genome (e.g., the reference genome of the subject). A respective methylation state vector is determined for each fragment, where the respective methylation state vector is associated with a location of the fragment in the reference genome (e.g., as specified by the position of the first CpG site in each fragment, or another similar metric) and comprises a number of CpG sites in the fragment as well as the methylation state of each CpG site in the fragment whether methylated (e.g., denoted as M), unmethylated (e.g., denoted as U), or indeterminate (e.g., denoted as I). Observed states are states of methylated and unmethylated; whereas, an unobserved state is indeterminate.

Those of skill in the art will appreciate that the principles described herein are equally applicable for the detection of methylation in a non-CpG context, including non-cytosine methylation. Further, methylation state vectors may contain elements that are generally vectors of sites where methylation has or has not occurred (even if those sites are not CpG sites specifically). With that substitution, the rest of the processes described herein are the same, and consequently the inventive concepts described herein are applicable to those other forms of methylation.

As used herein, the term “vector” is an enumerated list of elements, such as an array of elements, where each element has an assigned meaning. As such, the term “vector” as used in the present disclosure is interchangeable with the term “tensor.” As an example, if a vector comprises the bin counts for 10,000 bins, there exists a predetermined element in the vector for each one of the 10,000 bins. For ease of presentation, in some instances a vector may be described as being one-dimensional. However, the present disclosure is not so limited. A vector of any dimension may be used in the present disclosure provided that a description of what each element in the vector represents is defined (e.g., that element 1 represents bin count of bin 1 of a plurality of bins, etc.).

The terminology used herein is for the purpose of describing particular cases and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are used to implement a methodology in accordance with the features described herein.

### Exemplary System Embodiments.

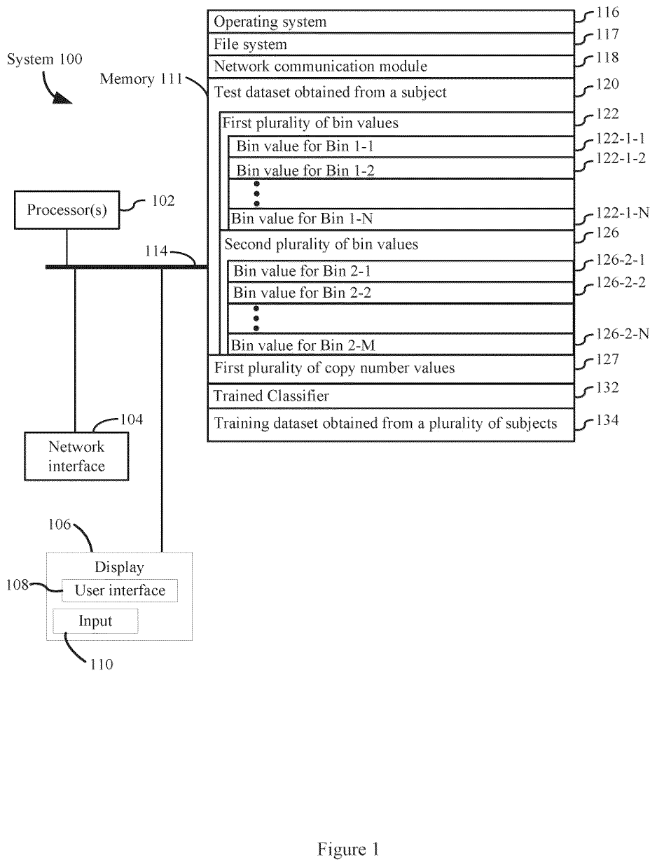

Details of an exemplary system are now described in conjunction with FIG. 1. FIG. 1 is a block diagram illustrating a system 100 in accordance with some implementations. The device 100 in some implementations includes at least one or more processing units CPU(s) 102 (also referred to as processors), one or more network interfaces 104 for connecting the device to a network, a display 106 having a user interface 108, an input device 110, a memory 111, and one or more communication buses 114 for interconnecting these components. The one or more communication buses 114 optionally include circuitry (sometimes called a chipset) that interconnects and controls communications between system components.

In some embodiments, each processing unit in the one or more processing units 102 is a single-core processor or a multi-core processor. In some embodiments, the one or more processing units 102 is a multi-core processor that enables parallel processing. In some embodiments, the one or more processing units 102 is a plurality of processors (single-core or multi-core) that enable parallel processing. In some embodiments, each of the one or more processing units 102 are configured to execute a sequence of machine-readable instructions, which can be embodied in a program or software. The instructions may be stored in a memory location, such as the memory 111. The instructions can be directed to the one or more processing units 102, which can subsequently program or otherwise configure the one or more processing units 102 to implement methods of the present disclosure. Examples of operations performed by the one or more processing units 102 can include fetch, decode, execute, and writeback. The one or more processing units 102 can be part of a circuit, such as an integrated circuit. One or more other components of the system 100 can be included in the circuit. In some embodiments, the circuit is an application specific integrated circuit (ASIC) or a field-programmable gate array (FPGA) architecture.

In some embodiments, the network is Internet, an internet and/or extranet, or an intranet and/or extranet that is in communication with the Internet. In some embodiments, the network 230 is a telecommunication and/or data network. In some embodiments, the network comprises one or more computer servers that can enable distributed computing, such as cloud computing. In some embodiments, the network, with the aid of the computer system 100, can implement a peer-to-peer network, which may enable devices coupled to the computer system 100 to behave as a client or a server. Such systems can be connected through a communications network to the Internet. The communications network can be any available network that connects to the Internet. The communications network can utilize, for example, a high-speed transmission network including, without limitation, Digital Subscriber Line (DSL), Cable Modem, Fiber, Wireless, Satellite and, Broadband over Powerlines (BPL). Examples of networks accessed by network interface 104 include, but are not limited to, the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In some embodiments, the display 106 is a touch-sensitive display, such as a touch-sensitive surface. In some embodiments, the user interface 106 includes one or more soft keyboard embodiments. In some implementations, the soft keyboard embodiments include standard (QWERTY) and/or non-standard configurations of symbols on the displayed icons. The user interface 106 may be configured to provide a user (e.g., health professionals) with graphic showings of, for example, results of targeted DNA methylation sequencing, disease conditions, and treatment suggestion or recommendation of preventive steps based on the disease conditions. The user interface may enable user interactions with particular tasks (e.g., reviewing the disease conditions and adjusting treatment plans).

The memory 111 may be a non-persistent memory, a persistent memory, or any combination thereof. The non-persistent memory can include high-speed random access memory, such as DRAM, SRAM, DDR RAM, ROM, PROM, EEPROM, flash memory, whereas the persistent memory typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. Regardless of its specific implementation, the memory 111 comprises at least one non-transitory computer readable storage medium, and it stores thereon computer-executable executable instructions which can be in the form of programs, modules, and data structures.

In some embodiments, as shown in FIG. 1, the memory 111 stores the following:
instructions, programs, data, or information associated with an operating system 116 (e.g., iOS, ANDROID, DARWIN, RTXC, LINUX, UNIX, OS X, WINDOWS, or an embedded operating system such as VxWorks), which includes various software components and/or drivers for controlling and managing general system tasks (e.g., memory management, storage device control, power management, etc.) and facilitates communication between various hardware and software components;
instructions, programs, data, or information associated with an optional file system 117 (which may be a component of operating system 116), for managing files stored or accessed by the system 100;
instructions, programs, data, or information associated with an optional network communication module 118 for connecting the system 100 with other devices and/or to a communication network;
a test dataset 120 obtained by targeted sequencing of a plurality of nucleic acids from a biological sample of a subject (e.g., a training subject or a test subject);
a first plurality of bin values 122 that can be included in the test dataset 120, each respective bin value (e.g., a bin value 122-1-1 for Bin 1-1, a bin value 122-1-2 for Bin 1-2, . . . , a bin value 122-1-N for Bin 1-N);
a second plurality of bin values 126 that can be included in the test dataset 120, each respective bin value (e.g., a bin value 126-2-1 for Bin 2-1, a bin value 126-2-2 for Bin 2-2, . . . , a bin value 126-2-N for Bin 2-N);
a plurality of copy number values 127 determined at least in part from the first plurality of bin values 122 or a combination of the first and second plurality of bin values 122/126;
instructions, programs, data, or information associated with a trained classifier 132 trained using a training dataset 134 comprising a plurality of copy number values derived from a plurality of subjects (e.g., training subjects), and an indication of a disease condition of each respective subject in the plurality of subjects; and
a training dataset 134 trained using data obtained from on-target regions and/or off-target regions from a plurality of subjects.

In various implementations, one or more of the above identified elements are stored in one or more of the previously mentioned memory devices, and correspond to a set of instructions for performing various methods described herein. In some embodiments, the above identified modules, data, or programs (e.g., sets of instructions) are not implemented as separate software programs, procedures, datasets, or modules, and thus various subsets of these modules and data may be combined or otherwise re-arranged in various implementations. In some implementations, the memory 111 optionally stores a subset of the modules and data structures identified above. Furthermore, in some embodiments, the memory stores additional modules and data structures not described above. In some embodiments, one or more of the above-identified elements is stored in a computer system, other than that of the system 100, that is addressable by the system 100 so that the system 100 may retrieve all or a portion of such data.

Although FIG. 1 depicts a “system 100,” the figure is intended as a functional description of the various features that may be present in computer systems than as a structural schematic of the implementations described herein. In practice, items shown separately can be combined and some items can be separate. Moreover, although FIG. 1 depicts certain data and modules in the memory 111 (which can be non-persistent or persistent memory), these data and modules, or portion(s) thereof, may be stored in more than one memory.

Methods as described herein can be implemented by way of machine (e.g., the one or more processing units 102) executable code stored on an electronic storage location of the computer system 100, such as, for example, on the memory 111. The machine executable or machine-readable code can be provided in the form of software. During use, the code can be executed by the one or more processing units 102. The code can be pre-compiled and configured for use with a machine having a processer adapted to execute the code, or can be compiled during runtime. The code can be supplied in a programming language that can be selected to enable the code to execute in a precompiled or as-compiled fashion.

Aspects of the systems and methods provided herein, such as the computer system 100, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links.

Methods and systems of the present disclosure can be implemented by way of one or more algorithms. An algorithm can be implemented by way of software upon execution by the one or more processing units 102. The algorithms can, for example, generate a pattern based on electrical signals received from one or more electrodes, such as a matrix of electrical signals, compare a pattern generated by the control system to one or more patterns associated with a reference or training population, make a confirmation of cancer condition, or any combination thereof, and others.

While a system in accordance with the present disclosure has been disclosed with reference to FIG. 1, methods in accordance with the present disclosure are now detailed. Any of the methods in accordance with embodiments of the present disclosure can make use of any of the assays, algorithms, or techniques, or combinations thereof, disclosed in U.S. patent application Ser. No. 15/793,830, filed Oct. 25, 2017 and/or International Patent Application No. PCT/US17/58099, filed Oct. 24, 2017, the content of each of which is hereby incorporated herein by reference in its entirety, in order to determine a cancer condition in a test subject or a likelihood that the subject has the cancer condition.

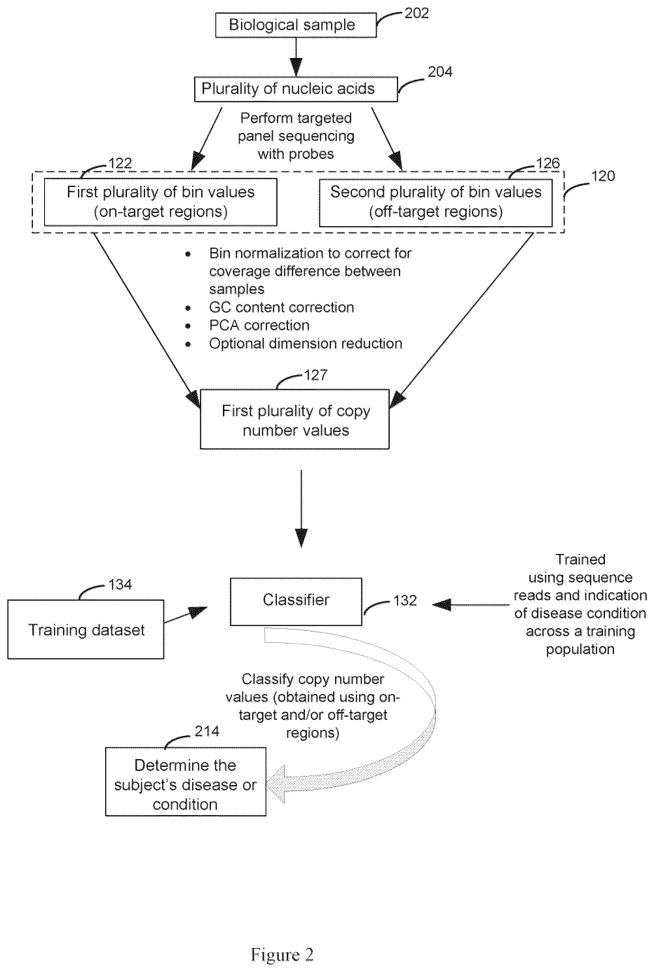

FIG. 2 illustrates an overview of the techniques in accordance with some embodiments of the present disclosure. In the described embodiments, a classifier is trained to determine whether a subject of a species has a disease condition in a set of disease conditions (e.g., a cancer condition). In some embodiments, the classifier is trained using bin values obtained from both on-target regions and off-target regions derived from a targeted sequencing of a plurality of nucleic acids from biological samples of a plurality of subjects. In this way, the present invention can improve computer technology by utilizing the generated sequencing data that is conventionally discarded and not used in analysis. The on-target regions can be identified as regions from the nucleic acids from the samples that correspond to a first plurality of bins defined for a reference genome of the species (e.g., using probes targeting sequences corresponding to those of the first plurality of bins), and off-target regions can be identified as regions from the nucleic acids from the samples that correspond to a second plurality of bins defined for the reference genome (e.g., sequences of the second plurality of bins are not targeted by sequences of the probes and thus result from accidental sequencing). In some embodiments, the second plurality of bins may partially overlap with the first plurality of bins. However, in other embodiments, the second plurality of bins do not overlap with the first plurality of bins. Moreover, in some such embodiments, not only do the second plurality of bins not overlap with the first plurality of bins, there is also a buffer between any bin in the first plurality of binds and any bin in the second plurality of bins. In some embodiments, the training dataset is obtained from the CCGA dataset (see Example 1). However, embodiments in accordance with the present disclosure can include any datasets in addition to specific datasets described herein.

In embodiments in which data obtained for on-target and off-target regions is combined for cancer/non-cancer prediction, the data can be combined by combining bin counts—for example, by combining features per bin (e.g., as a weighted sum, two-track input to a convolutional neural network, etc.). As another example, features (e.g., in the form of feature vectors) can be concatenated (e.g., as an example, 2× the features, 2 per bin), and PCA regression can then be applied to the concatenated features. In some embodiments, the combination can be performed by lengths of the sequence reads assigned to on-target and off-target bins, e.g., binned geometric mean of the cancer to non-cancer fragment length likelihood ratio. In some embodiments, on-target and off-target cancer and non-cancer length distributions are determined, and the lengths can be stratified by region. In some embodiments, features can be obtained separately for on-target and off-target, and the feature vectors are then concatenated.

As illustrated schematically in FIG. 2, methods are provided for inputting a test data set into the trained classifier to determine whether a subject of a species has a disease condition in a set of disease conditions. In some embodiments, for example, in which the disease condition is cancer, a type and/or stage of the disease (e.g., level of cancer) may be determined using the classifier. The techniques in accordance with the present disclosure can be implemented in any suitable computer system comprising at least one processor and a memory storing at least one program for execution by the at least one processor. For example, the method can be implemented at a computer system (e.g., computer system of FIG. 1) comprising at least one processor and a memory storing at least one program for execution by the at least one processor. The at least one program can comprise instructions that, when executed by the at least one processor, perform the described method.

As shown in FIG. 2, a biological sample 202 from a subject of a species (e.g., human) is processed to obtain a plurality of nucleic acids 204. In some embodiments, the nucleic acids 204 are cell-free nucleic acids. A targeted sequencing of the plurality of nucleic acids 204 is used to obtain a first plurality of bin values 122. In the first plurality of bin values 122, each respective bin value is for a corresponding bin in a first plurality of bins. Each bin in the first plurality of bins can represent a corresponding region of a reference genome of the species, and the first plurality of bins can collectively represent a first portion of the reference genome (e.g., the on-target regions). In some embodiments, the plurality of nucleic acids 204 is used to obtain a second plurality of bin values 126, e.g., based on the same targeted sequencing process. Alternatively, another plurality nucleic acids from the same subject can be used to generate the second plurality of bin values 126 in another sequencing process (e.g., targeted or non-targeted). An example of a non-targeted secondary sequencing process is whole genome sequencing. In the second plurality of bin values 126, each respective bin value is for a corresponding bin in a second plurality of bins. Each bin in the second plurality of bins can represent a corresponding region of a reference genome of the species, and the second plurality of bins can collectively represent a second portion of the reference genome (e.g., the off-target regions). Thus, the first portion of the genome may not be a contiguous portion of the genome. Likewise, the second portion of the genome may not be a contiguous portion of the genome. The first portion and the second portion of the genome may be formed from numerous disjointed portions of the reference genome. The bins for on-target regions can have sizes that are different from bin sizes of bins defined for off-target regions.

In the described embodiments, as indicated in FIG. 2, the plurality of nucleic acids 204 are enriched using a plurality of probes before the targeted sequencing. Each probe in the plurality of probes can include a nucleic acid sequence that corresponds to the sequence (or a portion thereof) of a bin in the first plurality of bins. Thus, a probe can align or substantially align (e.g., at least 70%, at least 75%, at least 80%, at least 85%, at least 90%, at least 95%, or at least 99% alignment) to the particular bin in the first plurality of bins. In some embodiments, a probe may align to more than one bin. In typical embodiments, a size of a probe is much smaller than a size of a bin.

In some embodiments, as shown in FIG. 2, a plurality of copy number values 127 are determined at least in part from the first and second plurality of bin values. In some embodiments, not shown in FIG. 2, the plurality of copy number values 127 are determined from the first plurality of bin values but not the second plurality of bin values. In still other embodiments, not shown in FIG. 2, some of the copy number values in the plurality of copy number values 127 are determined from the first plurality of bin values while other copy number values in the plurality of copy number values 127 are determined from the second plurality of bin values.

A copy number value can be derived from bin characteristics (bin values) that can be read counts, fragment lengths, fragment terminal positions, allelic imbalance measures, etc. The first and second plurality of bin values can be used to determine the copy number values 127 in various ways, using one or more mathematical transformations. In some embodiments, for example, the copy number values can be determined using fragment length metrics and/or fragment positioning metrics in the bin, as discussed in more detail below.

In the described embodiments, as mentioned above, both so-called “on-target” and “off-target” regions from the plurality of the nucleic acids 204, obtained using a targeted panel sequencing, may be used to determine the subject's disease or condition. The on-target region can be defined as a region that aligns or substantially aligns with a probe in a reference genome, whereas the off-target region can be defined as a region that does not align with a probe or aligns poorly with the probe. In other words, the off-target regions cannot be specifically sought, and they can be typically considered as “accidental” sequencing effects of the targeted panel sequencing. Embodiments of the present disclosure, however, utilize the off-target regions, together with on-target regions or even independently from the on-target regions, to use the signals in the off-target regions.

Accordingly, in some embodiments, the test dataset 120 further comprises a second plurality of bin values 126 that, like the first plurality of bin values 122, are derived from the targeted sequencing of the plurality of nucleic acids 204 from the biological sample 202 of the subject. The second bin values 126 can correspond to respective bins in a second plurality of bins, and each respective bin in the second plurality of bins can represent a corresponding region of the reference genome.

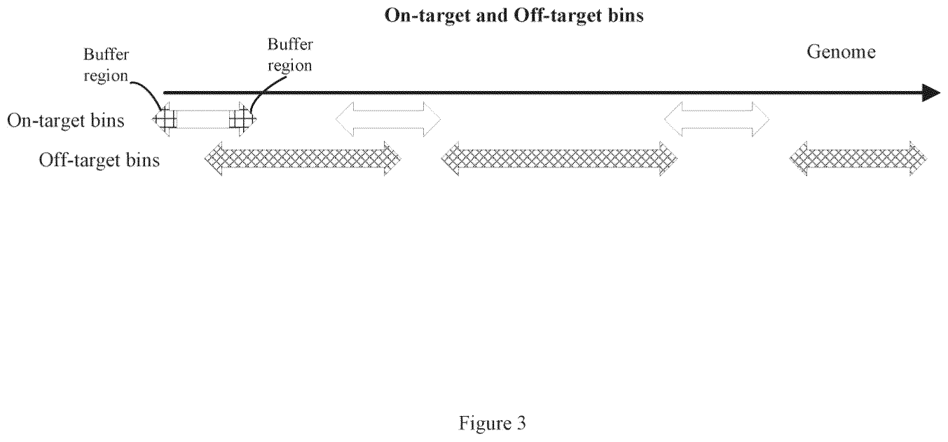

In some embodiments, the second plurality of bins collectively represent a second portion of the reference genome that does not overlap with the first portion represented by the first plurality of bins. However, in embodiments in which detection of copy number variants (CNV) and aberrations (CNA) from targeted sequencing data takes place, the first plurality and second plurality of bins can initially overlap. For example, in an embodiment, for off-target CNA, a whole genome can be divided into 20,000 or 30,000 bins of 100,000 kb, and the locations of sequence reads would fall into one of those bins. During processing of the genes that fall into the bins, however, sequence reads that map to a probe sequence (e.g., a location of a target gene, in some cases with padding) can be excluded from off-target regions. For on-target CNA, data corresponding to the second plurality of bins may be analyzed at a smaller scale, e.g., a size of the bin can be the size of a particular gene being targeted. In some embodiments, bins covering the on-target regions can be of the same or different sizes. The bins for on-target regions can have buffer regions (or padding) on both ends of the bin (e.g., about 200 bp). FIG. 3 illustrates schematically on-target and off-target bins defined for a reference genome. Bins covering the on-target regions can be of the same or different sizes.

In some embodiments, as shown in FIG. 2, prior to determining the copy number values 127, the described techniques include normalizing each respective bin value in the first and/or second plurality of bin values. The normalizing may involve one or more of various processing, including centering on a measure of central tendency within the sample, centering on data from a cohort of young and healthy reference subjects, normalization for GC content and principal component analysis (PCA) correction. Additionally or alternatively, the normalization may employ B-score processing. B-scores are described, for example, in U.S. patent application Ser. No. 16/352,739, entitled “Method and System for Selecting, Managing, and Analyzing Data of High Dimensionality,” filed Mar. 13, 2019, which is hereby incorporated by reference herein in its entirety. These normalizations (or corrections) can be performed in any order. The normalization may be performed to correct for differences in sequencing coverage between samples and/or to correct for differences across the plurality of patients. A PCA correction can be performed to reduce or eliminate variance in the sequencing data caused by potential confounding factors. In FIG. 2, such normalization is performed jointly on the first and second plurality of bin values. In other embodiments, separate normalization is performed on the first and second plurality of bin values.

In some embodiments, as illustrated in FIG. 2, in some embodiments the first and second plurality of bin values is subjected to dimension reduction. Thus, in such embodiments, the copy number values are in the form of reduced dimension components, such as, for example, principal components or another reduced dimension components. Thus, FIG. 2 illustrates that dimension reduction can be performed on the first and second plurality of bin values to thereby generate the plurality of copy number values that have reduced dimension. In FIG. 1, such dimension reduction is performed jointly on the first and second plurality of bin values to form the plurality of copy number values. For example, the first and second plurality of bin values can be combined and represented as one combined mathematical matrix (e.g., a rectangular array of numbers including one or more vectors) and the dimension reduction (e.g., PCA) can be performed on the combined mathematical matrix. In other embodiments, dimension reduction is separately performed on the first plurality of bin values and the second plurality of bin values (two separate dimension reductions, one for the first plurality of bin values to form some of the plurality of copy number values and another for the second plurality of bin values to form other of the plurality of copy number values) to form the plurality of copy number values. For example, the first plurality of bin values can be represented as a first mathematical matrix and the second plurality of bin values can be represented as a second mathematical matrix. In this situation, the dimension reduction can be separately performed on the first mathematical matrix and the second mathematical matrix.

Further, at least the plurality of copy number values can be inputted into a trained classifier 132, thereby determining (214) whether the subject has a disease condition in a set of disease conditions. The trained classifier 132 may be a neural network algorithm (e.g., a neural network algorithm a support vector machine algorithm (SVM), a Naive Bayes algorithm, a nearest neighbor algorithm, a random forest algorithm, a decision tree algorithm, a boosted trees algorithm, a regression algorithm, a logistic regression algorithm, a multi-category logistic regression algorithm, a linear discriminant analysis algorithm, or a clustering algorithm).

The trained classifier 132 can be trained using the training dataset 134 obtained from a plurality of subjects, and respective indications of a disease condition of each respective subject in the plurality of subjects. As discussed in more detail below, in some embodiments the classifier 132 is trained by obtaining the training dataset 134, that comprises, for each respective subject in the plurality of subjects, (i) a respective first plurality of bin values, each respective bin value in the first plurality of bin values for a corresponding bin in a first plurality of bins and (ii) a respective indication of the disease condition in the set of disease conditions for the respective subject. In some embodiments the classifier 132 is trained by obtaining the training dataset 134, that comprises, for each respective subject in the plurality of subjects, (i) a respective first plurality of bin values, each respective bin value in the first plurality of bin values for a corresponding bin in a first plurality of bins, (ii) a respective second plurality of bin values, each respective bin value in the second plurality of bin values for a corresponding bin in a second plurality of bins and (iii) a respective indication of the disease condition in the set of disease conditions for the respective subject. Each respective bin in the first plurality of bins can represent a corresponding region of a reference genome of the species. The first plurality of bins can collectively represent a first portion of the reference genome. Each respective bin in the second plurality of bins can represent a corresponding region of a reference genome of the species. The second plurality of bins can collectively represent a second portion of the reference genome. The respective first plurality of bin values and second plurality of bin values can be derived from a targeted sequencing of a plurality of nucleic acids from a biological sample of the respective subject using a plurality of probes that map to the first plurality of bins but not the second plurality of bins.

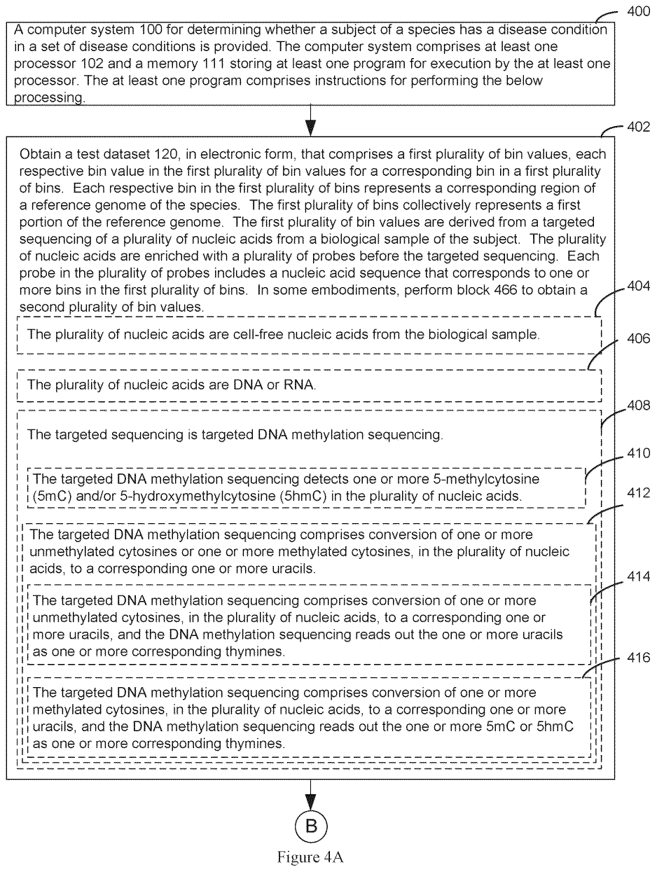

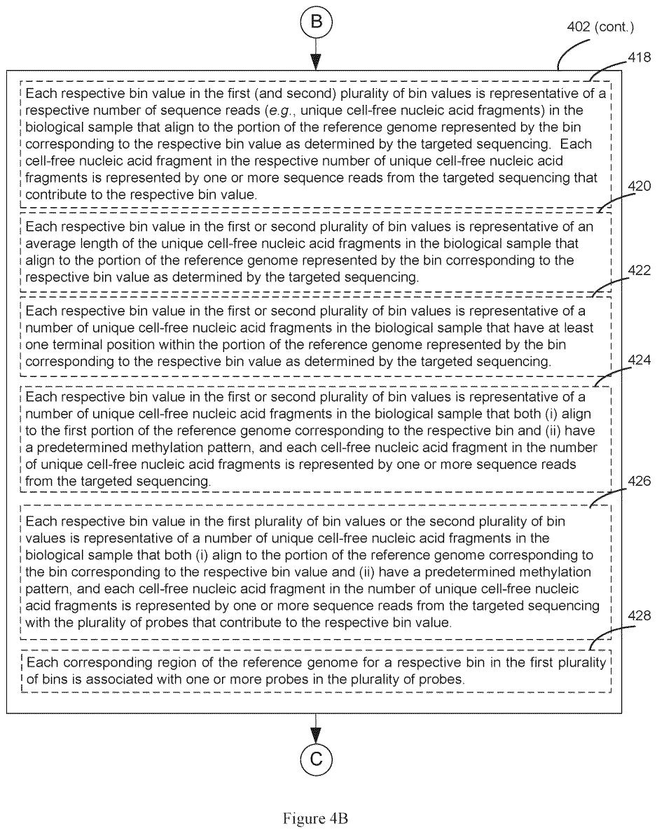

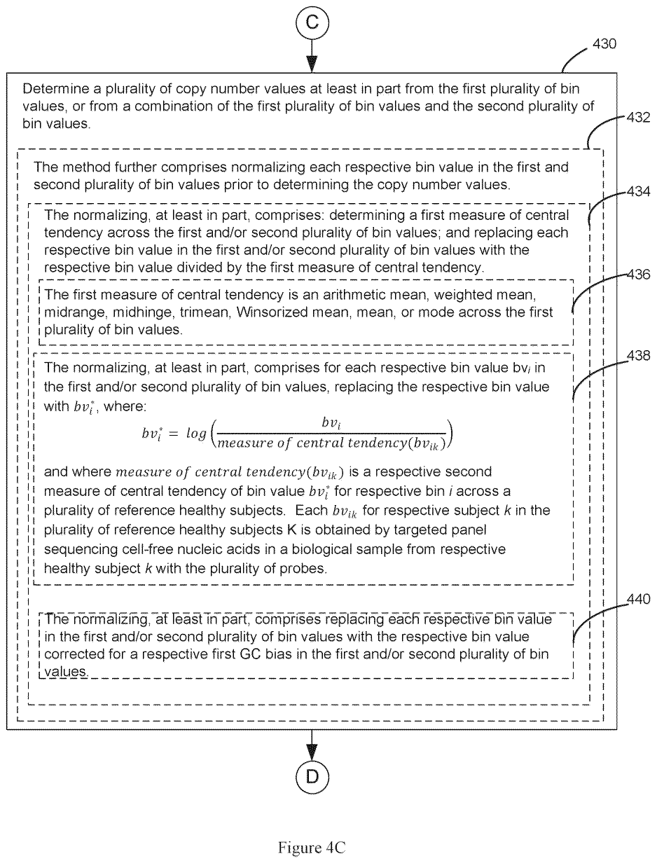

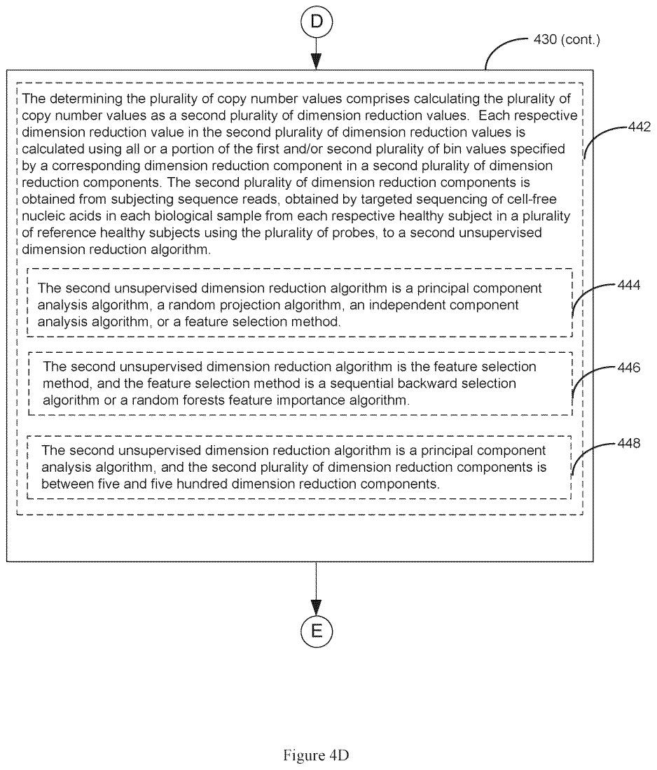

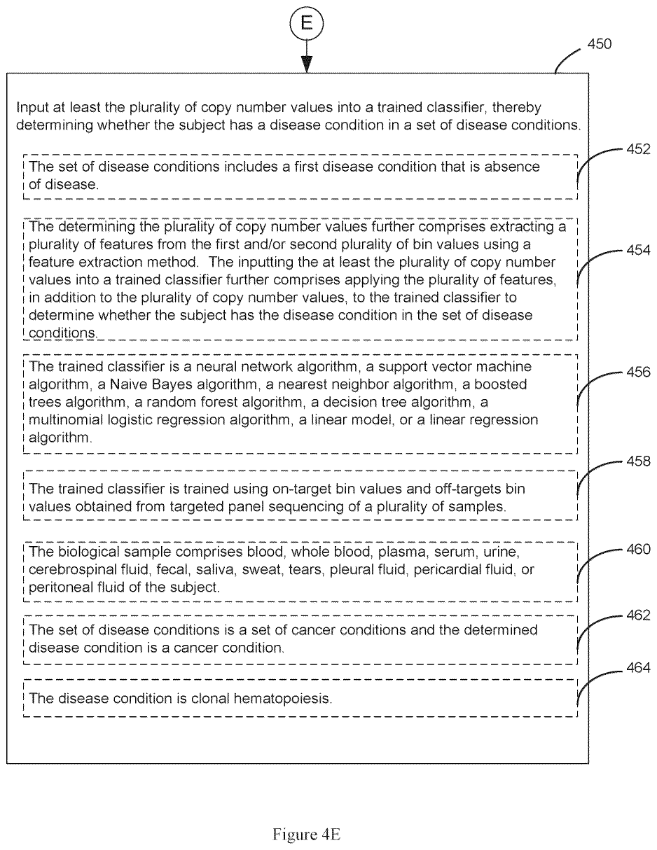

FIGS. 4A-4H illustrate an example of a method in accordance with some embodiments of the present disclosure.

### Blocks 400-416.

As shown at block 400, the method can be implemented by a computer system 100 for determining whether a subject of a species has a disease condition in a set of disease conditions. The computer system 100 comprises at least one processor 102 and a memory 111 storing at least one program for execution by the at least one processor. The at least one program can comprise instructions for performing the processing shown in FIGS. 4A-4H and described in detail below.

At block 402 of FIG. 4A, a test dataset is obtained, in electronic form, which comprises a first plurality of bin values, each respective bin value in the first plurality of bin values for a corresponding bin in a first plurality of bins. Each respective bin in the first plurality of bins can represent a corresponding region of a reference genome of the species. The first plurality of bins can collectively represent a first portion of the reference genome. The first plurality of bin values can be derived from a targeted sequencing of a plurality of nucleic acids from a biological sample of the subject. The plurality of nucleic acids can be enriched using a plurality of probes before the targeted sequencing. Each probe in the plurality of probes can include a nucleic acid sequence that corresponds to one or more bins in the first plurality of bins.

In some embodiments, a respective probe in the plurality of probes includes a corresponding nucleic acid sequence that is complementary or substantially complementary to the reference genome, or a portion thereof, as represented by a bin in the first plurality of bins with the exception of one or more nucleotide transitions. In some embodiments, each respective transition in the one or more transitions occurs at a respective un-methylated CpG dinucleotide site in the reference genome.

In some embodiments, a respective probe in the plurality of probes includes a corresponding nucleic acid sequence that is complementary or substantially complementary to the reference genome, or a portion thereof, as represented by a bin in the first plurality of bins with the exception of one or more nucleotide transitions. In some embodiments, each respective nucleotide transition in the one or more transitions occurs at a respective methylated CpG dinucleotide site in the reference genome.

In some embodiments, each probe in the plurality of probes includes a respective nucleic acid sequence that is complementary or substantially complementary to the reference genome, or a portion thereof, as represented by a bin in the first plurality of bins, with the exception that the probe includes an adenine to complement a thymine corresponding to a methylated or unmethylated cytosine in a selected cell-free nucleic acid (e.g., an original cell-free nucleic acid fragment).

In a reference genome, a significant percentage of CpG sites can be unmethylated (e.g., 95-97% of possible sites). In some embodiments, either methylated or unmethylated cytosines from CpG sites are converted (e.g., via a conversion treatment) to uracils in one or more target cell-free nucleic acid fragments (e.g., original cell-free nucleic acids). In such embodiments, after two or more rounds of PCR (e.g., performed as part of the sequencing analysis process), in the resulting sequence reads each such uracil from the original cell-free nucleic acid will be read as a thymine. In such embodiments, one or more probes in the plurality of probes may include an adenine as a complement to the resulting thymines.

In some embodiments, both on-target and off-target regions are used to determine whether or not a subject has a disease condition. Thus, as shown at block 466 of FIG. 4F, in some embodiments, a second plurality of bin values is also derived from the targeted sequencing of the plurality of nucleic acids from the biological sample of the subject. Each respective bin value in the second plurality of bin values can be for a corresponding bin in a second plurality of bins, each respective bin in the second plurality of bins can represent a corresponding region of the reference genome, and the second plurality of bins can collectively represent a second portion of the reference genome that does not overlap with the first portion.

As shown at block 404 of FIG. 4A, in some embodiments, the plurality of nucleic acids are cell-free nucleic acids from the biological sample. The plurality of nucleic acids can be DNA or RNA (block 406).

In some embodiments, the plurality of nucleic acids are obtained by whole genome sequencing or targeted panel sequencing of a biological sample from subjects. For example, the sequencing can be performed by whole genome sequencing with an average sequencing depth of at least 1×, 2×, 3×, 4×, 5×, 6×, 7×, 8×, 9×, 10×, at least 20×, at least 30×, or at least 40× across the genome of the test subject. In some embodiments, the sequencing depth for targeted panel sequencing can be much deeper, including but not limited to up to 1,000×, 2,000×, 3,000×, 5,000, 10,000×, 15,000×, 20,000×, or about 30,000×. In some embodiments, the sequencing depth can be deeper than 30,000×, e.g., at least 40,000× or 50,000×.

In some embodiments, the biological sample is blood. In some embodiments, the biological sample comprises whole blood, plasma, serum, urine, cerebrospinal fluid, fecal, saliva, sweat, tears, pleural fluid, pericardial fluid, or peritoneal fluid of the subject. In some embodiments, the biological sample consists of blood, whole blood, plasma, serum, urine, cerebrospinal fluid, fecal, saliva, sweat, tears, pleural fluid, pericardial fluid, or peritoneal fluid of the subject.

In some embodiments, the biological sample is processed to extract cell-free nucleic acids in preparation for sequencing analysis. In some embodiments, cell-free nucleic acid is extracted from a blood sample collected from a subject in K2 EDTA tubes. Samples can be processed within two hours of collection by double spinning of the blood first at ten minutes at 1000 g then plasma ten minutes at 2000 g. The plasma can then be stored in 1 ml aliquots at −80° C. In this way, a suitable amount of plasma (e.g., 1-5 ml) can be prepared from the biological sample for the purposes of cell-free nucleic acid extraction. In some such embodiments, cell-free nucleic acid is extracted using the QIAamp Circulating Nucleic Acid kit (Qiagen) and eluted into DNA Suspension Buffer (Sigma). In some embodiments, the purified cell-free nucleic acid is stored at −20° C. until use. See, for example, Swanton et al., 2017, “Phylogenetic ctDNA analysis depicts early stage lung cancer evolution,” Nature, 545(7655): 446-451, which is hereby incorporated herein by reference in its entirety. Other equivalent methods can be used to prepare cell-free nucleic acid using biological methods for the purpose of sequencing, and all such methods can be within the scope of the present disclosure.

In some embodiments, the cell-free nucleic acid that is obtained from the biological sample is in any form of nucleic acid, or a combination thereof. For example, in some embodiments, the cell-free nucleic acid that is obtained from a biological sample is a mixture of RNA and DNA.

The time between obtaining a biological sample and performing an assay, such as a sequence assay, can be optimized to improve the sensitivity and/or specificity of the assay or method. In some embodiments, a biological sample can be obtained immediately before performing an assay. In some embodiments, a biological sample can be obtained, and stored for a period of time (e.g., hours, days or weeks) before performing an assay. In some embodiments, an assay can be performed on a sample within 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, 1 year, or more than 1 year after obtaining the sample from a subject (e.g., a training subject).

In some embodiments, the nucleic acids are obtained by targeted panel sequencing in which the sequence reads taken from a biological sample of a subject in order to form a dataset comprising at least 50,000× sequencing depth for the portions of the genome to which the plurality of probes map, at least 55,000× sequencing depth for the portions of the genome to which the plurality of probes map, at least 60,000× sequencing depth for the portions of the genome to which the plurality of probes map, or at least 70,000× sequencing depth for the portions of the genome to which the plurality of probes map. In some such embodiments, the plurality of probes is between 50 and 5,000 probes, 50 and 4,000 probes, between 50 and 3,000 probes, between 50 and 2,000 probes, between 50 and 1,000 probes or between 50 and 500 probes. In some embodiments, each probe in the plurality of probes uniquely maps to a different gene. In some embodiments, a probe in the plurality of probes maps to a gene exon, a promoter region, or an enhancer region. In some embodiments, the plurality of probes is within a range of 500±5 probes, within a range of 500±10 probes, within a range of 500±25 probes or within a range of 500±100 probes.

In preferred embodiments, the first plurality of bin values and the second plurality of bin values are obtained from the same targeted panel sequencing process. That is, the same nucleic acids derived from the same sample can be used. As disclosed, a reference genome can be divided into on-target regions and off-target regions that are then used to group sequencing data accordingly: on-target sequencing data can be used to derive the first plurality of bin values while the off-target sequencing data can be used to derive the second plurality of bin values. As disclosed herein, the targeted panel sequencing can be non-methylation based or methylation-based. A non-limiting example of non-methylation based targeted panel sequencing is the ART sequencing assay that was performed on blood drawn from subjects in the CCGA study as described in Example 1.

In some embodiments, the second plurality of bin values can alternatively be obtained by a whole genome sequencing assay. A whole genome sequencing assay can refer to a physical assay that generates sequence reads for a whole genome or a substantial portion of the whole genome that can be used to determine large variations such as copy number variations or copy number aberrations. Such a physical assay may employ whole genome sequencing techniques or whole exome sequencing techniques.

In some embodiments, the second plurality of bin values can also be obtained by whole genome bisulfite sequencing. In some of such embodiments, the whole genome bisulfite sequencing identifies one or more methylation state vectors as described, for example, in U.S. patent application Ser. No. 16/352,602, entitled “Anomalous Fragment Detection and Classification,” filed Mar. 13, 2019, or in accordance with any of the techniques disclosed in U.S. Provisional Patent Application No. 62/847,223, entitled “Model-Based Featurization and Classification,” filed May 13, 2019, each of which is hereby incorporated by reference.

In some embodiments, bin values are determined from methylation sequencing information (e.g., bin values correspond to ratios of abnormally methylated fragments versus fragments having a methylation status matching the methylation status for a healthy control group); and in some such embodiments, bin values are determined using methylation state vectors as described in Example 5 in PCT/US2020/034317, entitled “Systems And Methods For Determining Whether A Subject Has A Cancer Condition Using Transfer Learning,” filed May 22, 2020, which is hereby incorporated by reference. In the present disclosure, the section below entitled “Protocol for obtaining methylation information from sequence reads of fragments in a biological sample” provides one example of first nucleic acid sequencing method in which methylation information is derived from the sequence reads and used to determine bin values.

In some embodiments, each bin value is a count of a number of cell-free nucleic acids from a biological sample that map to a bin. In some embodiments, this is determined through nucleic acid sequencing schemes that make use of a unique molecular identifier (UMI). That is, during the sequencing, each cell-free nucleic acid in a biological sample, and all the sequence reads that are derived from the cell-free nucleic acid, can be assigned the same UMI. Thus, all the sequence reads that have the same UMI can be considered to have been derived from a common cell-free nucleic acid (interchangeably referred to a “fragment”) and thus can be bagged into a single consensus sequence for the common cell-free nucleic acid. The term “bin value” can refer to any form of representation of the number of cell-free nucleic acids mapping to a given bin i. Such bin values can be in an un-normalized form (e.g., bvᵢ) or normalized form

$$( e . g . , {bv}_{i}^{*} , b {v}_{i}^{* *} , b {v}_{i}^{* * *} , b {v}_{i}^{* * * *} , etc . ) .$$

In some embodiments, unique cell-free nucleic acids (e.g., used for determining bin values) are determined by bagging PCR duplicates of sequence reads that have the same barcode (e.g., a UMI or unique molecular identifier). In some embodiments, when a cell-free nucleic acid overlaps multiple bins, it is assigned (contributes to the count) in each bin it overlaps. In some embodiments, when a cell-free nucleic acid overlaps multiple bins, it is assigned (contributes to the count) of the bin it overlaps the most.

In some embodiments, the first plurality of bins is derived from the sequences disclosed in Examples the sections below entitled “Example bins for methylation embodiments,” “Select human genomic regions used for bins,” Additional select human genomic regions used for bins, and/or “Additional Select human genomic regions used for bins.” In some such embodiments, adjacent and overlapping targets (genomic sequence targeted by a probe to a region disclosed in the sections below entitled “Example bins for methylation embodiments,” “Select human genomic regions used for bins,” Additional select human genomic regions used for bins, and/or “Additional Select human genomic regions used for bins”) are merged into contiguous genomic regions. In some embodiments, each of the resulting regions is used as-is as a corresponding bin in the first plurality of bins if smaller than a threshold number of base pairs (e.g., 1000 base pairs), or else subdivided into sub-regions (e.g., 1000 base pair regions).

In some embodiments, the first plurality of bins is derived such that each bin encompasses one, two, three, four, five, six, seven, or eight probes described in the section below entitled “Cancer assay probes and panels.” In some such embodiments, adjacent and overlapping targets (genomic sequence targeted by a probe in the section below entitled “Cancer assay probes and panels”) are merged into contiguous genomic regions. In some embodiments, each of the resulting regions is used as-is as a corresponding bin in the first plurality of bins if smaller than a threshold number of base pairs (e.g., 1000 base pairs), or else subdivided into sub-regions (e.g., 1000 base pair regions). Any positive integer value between 100 base pairs and 10 million base pairs can be used to define the first plurality of bins.

In some embodiments, the first plurality of bins is derived such that each bin encompasses a region of the genome described in the section below entitled “Example bins for methylation embodiments.” In some such embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps.

In some embodiments, the first plurality of bins is derived such that each bin encompasses a region of the genome described in the section below entitled “Select human genomic regions used for bins.” In some such embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps.

In some embodiments, the first plurality of bins is derived such that each bin encompasses a region of the genome described in the section below entitled “Additional select human genomic regions used for bins.” In some such embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps.

In some embodiments, the first plurality of bins is derived such that each bin encompasses a region of the genome described in the section below entitled “Additional Select human genomic regions used for bins.” In some such embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps.

In some embodiments, the first plurality of bins is derived from any combination of the bins disclosed in the sections entitled Example bins for methylation embodiments, “Select human genomic regions used for bins,” “Additional select human genomic regions used for bins,” or “Additional Select human genomic regions used for bins.” In some such embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps.

In some embodiments, each bin in the first plurality of bins represents all or a portion of an enhancer, promoter, 5′ UTR, exon, exon/inhibitor boundary, intron, intron/exon boundary, 3′ UTR region, CpG shelf, CpG shore, or CpG island in a reference genome. See, for example, Cavalcante and Santor, 2017, “annotatr: genomic regions in context,” Bioinformatics 33(15) 2381-2383, for suitable definitions of such regions and where such annotations are documented for a number of different species.

In some embodiments, each respective bin value is a measure of a frequency of abnormally methylated cell-free nucleic acids (e.g., cell-free nucleic acids including one or more abnormally methylated CpG sites) represented by the measured plurality of sequence reads that map to the genomic region represented by the corresponding bin.

In some embodiments, each respective bin value is determined from a methylation state vector derived from the first plurality of sequence reads that map to the genomic region represented by the corresponding bin. There are various ways to determine whether a specific cell-free nucleic acid (fragment) includes one or more abnormally methylated CpG sites. For example, U.S. patent application Ser. No. 16/719,902, entitled “Systems and Methods for Estimating Cell Source Fractions using Methylation Information,” filed Dec. 18, 2019, which is hereby incorporated by reference in its entirety, discloses methods for determining whether cell-free nucleic acids are abnormally methylated (e.g., by comparing methylation states for each respective cell-free nucleic acid to a reference dataset of methylation states—where the reference dataset is determined from the methylation states observed in a cohort of healthy reference subjects).

In some embodiments, each bin value indicates a respective copy number instability (CNI) for the corresponding bin. See Zhou et al. 2018 Bioinformatics 34(14), 2349-2355, which is hereby incorporated by reference, for an example method of how copy number score (e.g., here Z-score) may be calculated from bin count or bin value. In some embodiments, a bin value is in the form of a B-score, which is described, for example, in U.S. Patent Publication No. 2019-0287649, entitled “Method and System for Selecting, Managing, and Analyzing Data of High Dimensionality,” published Sep. 19, 2019, which is hereby incorporated by reference herein in its entirety.

In some embodiments, the plurality of nucleic acids are from training samples from the CCGA study, as described in Example 1 below. The plurality of nucleic acids can be processed to obtain copy number values, from on-target and off-target regions, that are used to train a classifier. A test dataset obtained from a biological sample from a subject can then be inputted into the trained classifier to determine whether the subject has a disease condition, and, in some embodiments, a type, stage and/or other characteristics of the disease condition.

In some embodiments, the sequencing method employs any form of targeted sequencing that can be used to obtain a number of sequence reads measured from cell-free nucleic acids. In some embodiments, such sequencing is performed on high-throughput sequencing systems such as the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used to obtain sequence reads 140 from the cell-free nucleic acid obtained from the biological sample.

In some embodiments, sequencing-by-synthesis and reversible terminator-based sequencing (e.g., Illumina's Genome Analyzer; Genome Analyzer II; HISEQ 2000; HISEQ 2500 (Illumina, San Diego Calif.)) are used to obtain sequence reads from the cell-free nucleic acid obtained from a biological sample of a subject, such as a training subject. In some such embodiments, millions of cell-free nucleic acid (e.g., DNA) fragments are sequenced in parallel. In one example of this type of sequencing technology, a flow cell is used that contains an optically transparent slide with eight individual lanes on the surfaces of which are bound oligonucleotide anchors (e.g., adaptor primers). A flow cell can be a solid support that is configured to retain and/or allow the orderly passage of reagent solutions over bound analytes. In some instances, flow cells are planar in shape, optically transparent, generally in the millimeter or sub-millimeter scale, and often have channels or lanes in which the analyte/reagent interaction occurs. In some embodiments, a cell-free nucleic acid sample can include a signal or tag that facilitates detection. In some such embodiments, the acquisition of sequence reads from the cell-free nucleic acid obtained from the biological sample includes obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

In some embodiments, where the sequencing assay is bisulfite sequencing, methylation state vectors are determined as disclosed in U.S. patent application Ser. No. 16/352,602, entitled “Anomalous Fragment Detection and Classification,” filed Mar. 13, 2019, or in accordance with any of the techniques disclosed in U.S. patent application Ser. No. 15/931,022, entitled “Model-Based Featurization and Classification,” filed May 13, 2020, each of which is hereby incorporated by reference. In such embodiments, a bin value reflects a number of fragments as represented by sequence reads that have a predetermined methylation state and that map onto the region of the reference genome corresponding to the respective bin. As an example, the bin value reflects methylation states based on the presence of CpG sites over a given length of nucleotide sequence.

In some embodiments, genomic regions with high variability or low mappability are excluded, for example, using the methods disclosed in Jensen et al, 2013, PLOS One 8; e57381. See also, Li and Freudenberg, 2014, Front. Genet. 5, p. 318, for analysis of mappability.

P-value filtering based on methylation vectors. In some embodiments, each cell-free nucleic acid in the plurality of cell-free nucleic acids used as part of determining bin counts has a corresponding p-value that is below a threshold value, where the p-value is determined by p-value filtering as described Example 5 in International Patent Application No. PCT/US2020/034317. The goal of such a filter condition can be to accept and use anomalously methylated cell-free nucleic acids for the determination of bin values based on their corresponding methylation state vectors. For example, for each cell-free nucleic acid (fragment) in a sample, a determination is made as to whether the fragment is anomalously methylated (e.g., via analysis of sequence reads derived therefrom), relative to an expected methylation state vector using the methylation state vector corresponding to the fragment (e.g., where the expected methylation state vector is determined from sequence analysis of a cohort (plurality) of healthy subjects). The generation of methylation state vectors for such cell-free nucleic acids (fragments) is disclosed, for example, in the section below entitled “Protocol for obtaining methylation information from sequence reads of fragments in a biological sample.” In some embodiments, the threshold value is 0.01 (e.g., p is <0.01 in such embodiments). In some embodiments, the threshold value is 0.001, 0,005, 0.01, 0.015, 0.02, 0.05, or 0.10. In some embodiments, the threshold value is between 0.0001 and 0.20. In such embodiments, those cell-free nucleic acids that have a p-value below the threshold value contribute to bin count. For example, in some embodiments, the plurality of cell-free nucleic acids is filtered by removing from the plurality of cell-free nucleic acids each respective cell-free nucleic acid whose corresponding methylation pattern (e.g. methylation state vector) across a corresponding plurality of CpG sites in the respective fragment has a p-value that fails to satisfy a p-value threshold.

In some embodiments, each cell-free nucleic acid (fragment) may have a bag-size greater than a threshold integer in order to contribute to a bin value. In other words, that each cell-free nucleic acid can be represented by more than the threshold integer of sequence reads in the plurality of sequence reads. For example, in the case where the threshold integer is one, each cell-free nucleic acid can be represented by more than one sequence read in the first plurality of sequence reads in order to contribute to a bin value. In some embodiments, the threshold integer is 1, 2, 3, 4, 5, 6, 7, 8, 9, 10, or an integer between 10 and 100.

In some embodiments, each cell-free nucleic acid covers a first threshold number of CpG sites and is less than a second threshold length in terms of base pairs in order to contribute to a bin value. For example, in the case where the first threshold is 1 CpG site and the second threshold 1000 base pairs, each cell-free nucleic acid can cover more than one CpG site and be less than 1000 base pairs in length in order to contribute to the bin that it maps to. In some embodiments, each cell-free nucleic acid can cover at least 1, 2, 3, 4, 5, 6, 7, 8, 9, 10, 11, 12, 13, 14, 15, 16, 17, 18, 19, or 20 CpG sites (e.g., within a particular nucleic acid length) in order to contribute to a bin value. In some embodiments, each cell-free nucleic acid can be less than 500, 1000, 2000, 3000, or 4000 contiguous base pairs in length in order to contribute to a bin value. In other words for example, in some embodiments, each cell-free nucleic acid that contributes to a bin count includes at least 1 CpG site, at least 2 CpG sites, at least 3 CpG sites, at least 4 CpG sites, at least 5 CpG sites, at least 6 CpG sites, at least 7 CpG sites, at least 8 CpG sites, at least 9 CpG sites, at least 10 CpG sites, at least 11 CpG sites, at least 12 CpG sites, at least 13 CpG sites, at least 14 CpG sites, or at least 15 CpG sites within less than 500 contiguous nucleotides of the reference genome in some embodiments.

In some embodiments, each fragment is hypermethylated in order to contribute to a bin value. In some embodiments, each cell-free nucleic acid is hypomethylated in order to contribute to a bin value. In some embodiments, the filter condition is bin dependent. For instance, International Patent Publication No. WO2019/195268, entitled “Methylation Markers and Targeted Methylation Probe Panels,” filed Apr. 2, 2019, which is hereby incorporated by reference, discloses a number of regions of the human genome that have a hypermethylated state that is associated with one or more cancer conditions as well as a number of regions of the human genome that have a hypomethylated that is associated with one or more cancer conditions. Accordingly, in some embodiments of the present disclosure one or more bins in the first plurality of bins each represent a corresponding genomic region in the regions disclosed in WO2019/19528 and the filter condition in the plurality of filter conditions (a) includes selection of cell-free nucleic acids that are hypermethylated when selecting cell-free nucleic acids that map to a bin representing a region of the human genome that has a hypermethylated state that is associated with one or more cancer conditions of CpG sites as indicated by WO2019/195268 and (b) includes selection of cell-free nucleic acids that are hypomethylated when selecting fragments that map to a bin representing a region of the human genome that has a hypomethylated state that is associated with one or more cancer conditions of CpG sites as indicated by WO2019/195268.

In some embodiments, bin counts are determined using any of the techniques disclosed in U.S. patent application Ser. No. 16/201,912 entitled “Models for Targeted Sequencing,” filed Nov. 27, 2018 or U.S. patent application Ser. No. 16/352,214 entitled “Identifying Copy Number Aberrations,” filed Mar. 13, 2019, each of which is hereby incorporated by reference in its entirety.

Referring back to FIG. 4A, in some embodiments, the targeted sequencing is targeted DNA methylation sequencing (block 408). The targeted DNA methylation sequencing can be performed in various ways. Different enzymatic treatments and combination with chemical treatment(s) can convert either methylated cytosines or unmethylated cytosines. For example, in some embodiments, the targeted DNA methylation sequencing detects one or more 5-methylcytosine (5mC) and/or 5-hydroxymethylcytosine (5hmC) in the plurality of nucleic acids (block 410). As another example, the targeted DNA methylation sequencing may comprise conversion of one or more unmethylated cytosines or one or more methylated cytosines, in the plurality of nucleic acids, to a corresponding one or more uracils (block 412). As another example, as shown at block 414, in some embodiments, the targeted DNA methylation sequencing may comprise conversion of one or more unmethylated cytosines, in the plurality of nucleic acids, to a corresponding one or more uracils, and the DNA methylation sequence reads out the one or more uracils as one or more corresponding thymines. In some embodiments, the targeted DNA methylation sequencing comprises conversion of one or more methylated cytosines, in the plurality of nucleic acids, to one or more corresponding uracils, and the DNA methylation sequence reads out the one or more 5mC or 5hmC as one or more corresponding thymines (block 416).

### Blocks 418-428.

In the described embodiments, a bin value for a bin, representing a portion of a reference genome, can be determined in various ways, e.g., based on sequence read counts, fragment lengths, fragment terminal positions, etc. For example, in some embodiments, a bin value can be determined based on a read count. For example, in some embodiments, as shown at block 418 of FIG. 4B, each respective bin value in the first plurality of bin values and the second plurality of bin values is representative of a respective number of sequence reads in the biological sample that align to the portion of the reference genome represented by the bin corresponding to the respective bin value as determined by the targeted sequencing.

In some embodiments, a number of unique cell-free nucleic acid fragments, which align to the portion of the reference genome represented by the bin, can be used. In such embodiments, each cell-free nucleic acid fragment in the respective number of unique cell-free nucleic acid fragments is represented by one or more sequence reads from the targeted sequencing that contribute to the respective bin value. Accordingly, in some embodiments, a unique molecular identifier (UMI) is added to each fragment of cell-free nucleic acid in a plurality of cell-free nucleic acids in the biological sample prior to sequencing to ensure that bin counts are counts of individual cell-free nucleic acids in the biological sample (termed “fragments”), rather than duplicates of such cell-free nucleic acids that arise during the sequencing. In some embodiments, each such UMI is a unique nucleic acid sequence.

In some embodiments, multiple bin values can be determined for a bin, each based on sequencing data that align to a region of a reference genome represented by the bin and correspond to nucleic acid fragments of a particular length or length range. For example, instead of a linear array, a multidimensional array can be used to represent sequencing data from the on-target regions and/or off-target regions. Alternatively, as shown at block 420, in some embodiments, each respective bin value in the first plurality of bin values or the second plurality of bin values can be representative of an average length of the unique cell-free nucleic acid fragments in the biological sample that align to the portion of the reference genome represented by the bin corresponding to the respective bin value as determined by the targeted sequencing.

In some embodiments, a bin value for a bin is determined based on a number of fragments with a terminal position falling within that bin. Such an example is shown with reference to block 422, in which each respective bin value in the first or second plurality of bin values may be representative of a number of unique cell-free nucleic acid fragments in the biological sample that have at least one terminal position within the portion of the reference genome represented by the bin corresponding to the respective bin value as determined by the targeted sequencing.

The bin value can be determined in various other ways. For example, with reference to block 424 of FIG. 4B, each respective bin value in the first or second plurality of bin values may be representative of a number of unique cell-free nucleic acid fragments in the biological sample that both (i) align to the first portion of the reference genome corresponding to the respective bin and (ii) have a predetermined methylation pattern. In such embodiments, each cell-free nucleic acid fragment in the number of unique cell-free nucleic acid fragments may be represented by one or more sequence reads from the targeted sequencing.

Further, in some embodiments, at shown at block 426, each respective bin value in the first or second plurality of bin values is representative of a number of unique cell-free nucleic acid fragments in the biological sample that both (i) align to the portion of the reference genome corresponding to the bin corresponding to the respective bin value and (ii) have a predetermined methylation pattern. Each cell-free nucleic acid fragment in the number of unique cell-free nucleic acid fragments may be represented by one or more sequence reads from the targeted sequencing with the plurality of probes that contribute to the respective bin value.

Regardless of the specific way in which a bin value is determined, in some embodiments, each corresponding region of the reference genome for a respective bin in the first plurality of bins is associated with one or more probes in the plurality of probes, as shown at block 428 of FIG. 4B. Thus, these regions are targeted regions that may correspond to one probe, a probe set, or more than one probe sets. In some embodiments, the probes may be designed such that they bind to sequences after cytosines in methylated CpG sites or un-methylated CpG sites are converted (e.g., in a chemical or enzymatic conversion process). In embodiments in which methylation sequencing is used, sequences of the probes may not be complementary to the corresponding genomic sequence but rather to the sequences of the converted DNA fragments.

In some embodiments, the first portion of the reference genome may collectively encompass between 0.5 megabase and 50 megabases of unique sequences in the reference genome. The first portion of the reference genome may encompass other ranges of the reference genome—for example, in some embodiments, the range may be between 1 megabase and 40 megabases, between 4 megabases and 30 megabases, between 15 megabases and 35 megabases, between 20 megabases and 30 megabases, between 25 megabases and 35 megabases, between 30 megabases and 40 megabases, etc. The sequences that fall within the first portion of the reference genome may not be contiguous.

In some embodiments, the second plurality of bins represents a second portion of the reference genome. In some embodiments, the second portion of the reference genome collectively encompasses between 1 megabase and 50 megabases of unique sequences in the reference genome. The second portion of the reference genome may encompass other ranges of the reference genome—for example, in some embodiments, the range may be between 5 megabases and 40 megabases, between 10 megabases and 30 megabases, between 15 megabases and 35 megabases, between 20 megabases and 30 megabases, between 25 megabases and 35 megabases, between 30 megabases and 40 megabases, etc.

In some embodiments, the plurality of probes consists of between 1,000 and 2,000,000 probes. In some embodiments, the plurality of probes consists of between 500 and 2,000,000 probes. In some embodiments, the plurality of probes comprises more than 2,000,000 probes. In some embodiments, the plurality of probes consists of between 1000 and 1,500,000 probes. In some embodiments, the plurality of probes consists of between 1000 and 1,400,000 probes. In some embodiments, the plurality of probes consists of between 1000 and 1,300,000 probes. In some embodiments, the plurality of probes consists of between 1000 and 1,200,000 probes. In some embodiments, the plurality of probes consists of between 1000 and 1,100,000 probes. In some embodiments, the plurality of probes consists of between 1000 and 1,000,000 probes. In some embodiments, the plurality of probes consists of between 1000 and 900,000 probes. In some embodiments, the plurality of probes consists of between 1000 and 800,000 probes. In some embodiments, the plurality of probes consists of between 1000 and 700,000 probes. In some embodiments, the plurality of probes consists of between 1000 and 600,000 probes. In some embodiments, the plurality of probes consists of between 1000 and 500,000 probes. In some embodiments, the plurality of probes consists of between 1000 and 400,000 probes. In some embodiments, the plurality of probes consists of between 1000 and 300,000 probes. In some embodiments, the plurality of probes consists of between 1000 and 200,000 probes. In some embodiments, the plurality of probes consists of between 1000 and 100,000 probes. In some embodiments, the plurality of probes consists of between 1000 and 90,000 probes. In some embodiments, the plurality of probes consists of between 1000 and 80,000 probes. In some embodiments, the plurality of probes consists of between 1000 and 70,000 probes. In some embodiments, the plurality of probes consists of between 1000 and 60,000 probes. In some embodiments, the plurality of probes consists of between 1000 and 50,000 probes. In some embodiments, the plurality of probes consists of between 1000 and 40,000 probes. In some embodiments, the plurality of probes consists of between 1000 and 30,000 probes. In some embodiments, the plurality of probes consists of between 1000 and 20,000 probes. In some embodiments, the plurality of probes consists of between 1000 and 10,000 probes. In some embodiments, the plurality of probes consists of between 1000 and 9,000 probes. In some embodiments, the plurality of probes consists of between 1000 and 8,000 probes. In some embodiments, the plurality of probes consists of between 1000 and 7,000 probes. In some embodiments, the plurality of probes consists of between 1000 and 6,000 probes or fewer. In some embodiments, the plurality of probes consists of between 1000 and 5,000 probes or fewer. In some embodiments, the plurality of probes consists of between 1000 and 4,000 probes or fewer. In some embodiments, the plurality of probes consists of between 1000 and 3,000 probes. In some embodiments, the plurality of probes consists of between 1000 and 2,000 probes. In some embodiments, the plurality of probes consists of between 100 and 900 probes.

In some embodiments, at least one probe is designed to bind and enrich nucleic acids in the biological sample that contain at least one predetermined CpG site. In some implementations, each probe can be designed to bind and enrich nucleic acids in the biological sample that contain at least one predetermined CpG site.

A probe can be designed for targeting nucleic acids that have a certain number of predetermined CpG sites. For example, in some embodiments, one or more probes in the plurality of probes are designed to bind and enrich nucleic acids in the biological sample that contain 50 or fewer predetermined CpG sites, 40 or fewer predetermined CpG sites, 30 or fewer predetermined CpG sites, 25 or fewer predetermined CpG sites, 22 or fewer predetermined CpG sites, 20 or fewer predetermined CpG sites, 18 or fewer predetermined CpG sites, 15 or fewer predetermined CpG sites, 12 or fewer predetermined CpG sites, 10 or fewer predetermined CpG sites, 5 or fewer predetermined CpG sites, 3 or fewer predetermined CpG sites.

The bins in the first plurality of bins (e.g., on-target bins) can cover various regions in the reference genome, including the regions that are not contiguous. In some embodiments, each bin in the first plurality of bins does not overlap with another bin in the first plurality of bins. The bins can have various sizes. For example, a bin in the first plurality of bins can have between about 10 and about 10,000 nucleotides (nt), between about 10 and about 5,000 nt, between about 10 and about 2,000 nt, between about 10 and about 1,000 nt, between about 50 and about 500 nt, or between about 100 and about 250 nt. In some embodiments, each bin has about 150 nt, or fewer than 150 nt.

### Blocks 430-444.

In some embodiments, with reference to block 430 of FIG. 4C, a plurality of copy number values is determined at least in part from the first plurality of bin values or from a combination of the first plurality of bin values and the second plurality of bin values.

In some embodiments, all of the copy number values are determined from a combination of the first and second plurality of bin values. In other embodiments, a first subset of the copy number values are determined from the first plurality of bin values and a second subset, other than the first subset, of the copy number values are determined from the second plurality of bin values. In still other embodiments, a first subset of the copy number values is determined from the first plurality of bin values, a second subset of the copy number values is determined from the second plurality of bin values, and a third subset of the copy number values is determined from a combination of the first and second plurality of bin values.

The plurality of copy number values can be determined in various ways. A copy number value can be derived from bin characteristics such as, for example, sequence read counts, an average length of fragments assigned to the bin, end positions of fragments assigned to the bin, as well as other fragment length metrics and fragment positioning metrics measured with respect to the bin. The plurality of copy number values can be determined using various mathematical transformations.

The plurality of bin values may include heterogeneous data such that some form of normalization may be useful to extract meaningful signals from the bin values. Accordingly, in some embodiments, each respective bin value in the first and second plurality of bin values is normalized prior to the determining the plurality of copy number values, as shown at block 432. The normalizing can be performed in various ways. For example, the normalizing can include centering the first and second plurality of bin values on a measure of central tendency within the biological sample, centering the first and second plurality of bin values on bin values obtained from a cohort of young healthy subjects, performing GC content correction, PCA (principal component analysis)-based adjustment, and/or performing any other type(s) of normalization.

More than one type of normalization can be applied, and the normalization techniques can be applied in any suitable order. Moreover, the normalization can be separately applied to the first and second plurality of bin values or it can be applied on a combination of the first and second bin values. For example, in some embodiments, the normalization may involve, in this order, centering the first and/or second plurality of bin values on a measure of central tendency within the sample, centering the first and/or second plurality of bin values on bin values obtained from a cohort of young healthy subjects, performing GC correction, and performing PCA correction.

Accordingly, in some embodiments, as shown at block 434, the normalizing, at least in part, comprises determining a first measure of central tendency across the first and/or second plurality of bin values, and replacing each respective bin value in the first and/or second plurality of bin values with the respective bin value divided by the first measure of central tendency. The measure of central tendency may be an arithmetic mean, weighted mean, midrange, midhinge, trimean, Winsorized mean, mean, or mode across the first plurality of bin values, as shown at block 436 of FIG. 4C.

In some embodiments, additionally or alternatively, the normalization includes centering the first and/or second plurality of bin values based on information obtained from a cohort of young healthy subjects. In this way, in an embodiment, the normalization can be performed such that a positive bin value indicates amplification relative to the healthy cohort, and a negative bin value indicates a deletion relative to the healthy cohort.

With reference to block 438 of FIG. 4C, in some embodiments, the normalizing, at least in part, may comprise, for each respective bin value bvᵢ in the first and/or second plurality of bin values, replacing the respective bin value with

$$b {v}_{i}^{*} ,$$

where:

$$b {v}_{i}^{*} = log ( \frac{b {v}_{i}}{measure of central tendency ( {bv}_{i k} )} )$$

and where measure of central tendency (bvᵢₖ), where k runs from 1 to K (K being number of subjects in the cohort of young healthy subjects), is a respective second measure of central tendency of bin value

$$b {v}_{i}^{*}$$

for respective bin i across a plurality of reference healthy subjects. Each bvᵢₖ for respective subject k in the plurality of reference healthy subjects can be obtained by targeted panel sequencing cell-free nucleic acids in a biological sample from respective healthy subject k with the plurality of probes. The respective second measure of central tendency can be an arithmetic mean, weighted mean, midrange, midhinge, trimean, Winsorized mean, mean, or mode of bin value

$$b {v}_{i}^{*}$$

for respective bin i across the plurality of reference healthy subjects.

In some embodiments, with reference to block 440, the normalizing, at least in part, may further comprise replacing each respective bin value in the first and/or second plurality of bin values with the respective bin value corrected for a respective first GC bias in the first and/or second plurality of bin values. The respective first GC bias may be defined by a first equation for a curve or line fitted to a first plurality of two-dimensional points. Each respective two-dimensional point in the first plurality of two-dimensional points may include (i) a first value that is the respective GC content of the corresponding portion of the reference genome of the species represented by the respective bin in the first and/or second plurality of bins corresponding to the respective two-dimensional point and (ii) a second value that is the bin value in the first and/or second plurality of bin values for the respective bin. The replacing each respective bin value in the first and/or second plurality of bin values with the respective bin value corrected for a respective first GC bias in the first and/or second plurality of bin values may comprise subtracting a predicted GC bias for the respective bin, derived by inputting the proportion of G and C bases of the corresponding portion of the reference genome represented by the respective bin into the first equation, from the respective bin value. The correction for GC content bias can be performed as described, for example, in WO2013052913, U.S. Pat. No. 10,095,831, US20160239604, and in Benjamini and Speed, 2012, “Summarizing and correcting the GC content bias in high-throughput sequencing,” Nucleic Acids Res. 40(10), each of which is incorporated by reference herein in its entirety.

In some embodiments, a normalization (or a standardization) of the first and/or second plurality of bin values may be performed by using an unsupervised dimension reduction algorithm, also referred to herein as a first unsupervised dimension reduction algorithm. For example, a PCA correction may be performed in such manner. In these embodiments, such normalizing, at least in part, comprises, for each respective bin value

$$b {v}_{i}^{* *}$$

in the first and/or second plurality of bin values, replacing the respective bin value with

$$b {v}_{i}^{* * *} ,$$

where:

$$b {v}_{i}^{* * *} = b {v}_{i}^{* *} - \hat{b} {v}_{i}^{* *}$$

and where

$$\hat{b} {v}_{i}^{* *}$$

is a linear function of PC₁, . . . , PC_{N}, obtained by fitting a linear model over top principal components, N is a positive integer between 2 and 50, and PC₁, . . . , PC_{N} are a top number of dimension reduction components in a first plurality of dimension reduction components derived from subjecting respective normalized bin values for the first and/or second plurality of bins to a first unsupervised dimension reduction algorithm.

The bin values for the first and/or second plurality of bins can be obtained from targeted sequencing of each respective biological sample from each respective healthy subject in a plurality of reference healthy subjects, and the nucleic acids from the respective biological sample may have been enriched using the plurality of probes before sequencing analysis. The normalization of the bin values may include a suitable technique, including a sample normalization, baseline normalization, GC correction, or any combination thereof. In some embodiments, N is between three and ten. N can be a positive integer within any other range.

In some embodiments, the first and/or second plurality of bin values are normalized PCA to remove higher-order artifacts for a population-based correction. See, for example, Price et al., 2006, Nat Genet 38, pp. 904-909; Leek and Storey, 2007, PLOS Genet 3, pp. 1724-1735; and Zhao et al., 2015, Clinical Chemistry 61(4), pp. 608-616. Such normalization can be in addition to or instead of any of the above-identified normalization techniques. In some such embodiments, to train the PCA normalization, a data matrix comprising LOESS normalized bin counts

$$b {v}_{i}^{* * *}$$

from young healthy subjects in the plurality of reference healthy subjects (or another cohort that was sequenced in the same manner as the subject whose disease or condition is to be determined) is used and the data matrix is transformed into principal component space thereby obtaining the top N number of principal components across the training set. In some embodiments, the top 2, the top 3, the top 4, the top 5, the top 6, the top 7, the top 8, the top 9, the top 10, or more than the top 10 such principal components are used to build a linear regression model. The top principal components represent a common bias that can be modeled using samples from healthy controls (or a healthy cohort), and therefore removing such common bias (in the form of the top principal components derived from the healthy cohort) from the bin values

$$b {v}_{i}^{* * *}$$

can effectively improve normalization. See Zhao et al., 2015, Clinical Chemistry 61(4), pp. 608-616 for further disclosure on PCA normalization of sequence reads using a health population. Regarding the above normalization, variables may be standardized (e.g., by subtracting their means and dividing by their standard deviations).

Throughout the present disclosure, the term “bin value” can refer to any form of representation of the number of nucleic fragments mapping to a given bin i, and that such bin value can be in un-normalized (e.g., bvᵢ) or normalized form

$$( e . g . , b {v}_{i}^{*} , b {v}_{i}^{* *} , b {v}_{i}^{* * *} , b {v}_{i}^{* * * *} , etc . ) .$$

Accordingly, the first unsupervised dimension reduction algorithm may be a PCA algorithm, a random projection algorithm, an independent component analysis algorithm, or a feature selection method. In embodiments in which a PCA is used as a dimension reduction algorithm, a first plurality of dimension reduction components may be in the form of principal components. A certain number of principal components can be retained for further analysis. In some embodiments, the first unsupervised dimension reduction algorithm is the feature selection method, and the feature selection method is a sequential forward or backward selection algorithm.

As mentioned above, a probe (which can be referred to as an enrichment probe) used in a targeted panel sequencing, employed in accordance with the present disclosure, can include a respective nucleic acid sequence that is identical, nearly identical, or substantially identical to a portion of the reference genome or its reverse complement.

Thus, in some embodiments in accordance with the present disclosure, each respective probe in the plurality of probes includes a respective nucleic acid sequence that is identical, nearly identical, or substantially identical to a portion of the reference genome or its reverse complement, as represented by a bin in the first plurality of bins. The probe can be defined as “nearly identical” to a portion of the reference genome or its reverse complement when the probe is at least 98% identical to the portion of the reference genome or its reverse complement. The probe can be defined as “substantially identical” to a portion of the reference genome or its reverse complement when the probe is at least 85% identical to the portion of the reference genome or its reverse complement.

In some embodiments, a respective probe in the plurality of probes includes a respective nucleic acid sequence that is identical, nearly identical, or substantially identical to a portion of the reference genome or its reverse complement, as represented by a bin in the first plurality of bins with the exception of one or more transitions. Each respective transition in the one or more transitions may occur at a respective un-methylated CpG dinucleotide site in the reference genome.

As another example, in some embodiments, a respective probe in the plurality of probes includes a respective nucleic acid sequence that is identical, nearly identical, or substantially identical to a portion of the reference genome or its reverse complement, as represented by a bin in the first plurality of bins with the exception of one or more transitions, and each respective transition in the one or more transitions occurs at a respective methylated CpG dinucleotide site in the reference genome.

In some embodiments, the described techniques involve subjecting the plurality of nucleic acids from a biological sample of the subject to a conversion treatment, prior to obtaining the test dataset at block 402 of FIG. 4A. The conversion treatment may cause one or more unmethylated cytosines in the plurality of nucleic acids to be converted to one or more corresponding bases, or the conversion treatment may cause one or more methylated cytosines in the plurality of nucleic acids to be converted to one or more corresponding bases. For example, in some embodiments, as described in more detail below, the plurality of nucleic acids from a biological sample of the subject are subjected to a conversion treatment, prior to obtaining the test dataset comprising plurality of bin values. In such embodiments, the probes are designed to be complementary to the converted sequences, and the probes therefore may be partially complementary to the reference genome. As an illustrative example, for an original DNA molecule (1) ATCGATCGCTAGATCCATCG (SEQ ID.: No. 1) including three CpG sites, one may be methylated (e.g., 95% of the cytosines in the genome sites are not methylated). Accordingly, after bisulfite treatment, the sequence is read out as (2) ATCGATIGCTAGATCCATTG (SEQ ID.: No. 2), such that the methylated C is read out as C, whereas the other Cs are read out as T; e.g., the underlined nucleotides in sequence (2). In this example, an enrichment probe may have a sequence that is complementary to the sequence (2) rather than to the sequence (1).

In some embodiments, the described method for determining whether a subject of a species has a disease condition in a set of disease condition further comprises, prior to the step of obtaining the test dataset (block 402 of FIG. 4A), subjecting the plurality of nucleic acids to a bisulfite conversion treatment, thereby causing one or more unmethylated cytosines in the plurality of nucleic acids to be converted to one or more corresponding uracils. In these embodiments, the targeted sequencing of the plurality of nucleic acids reads out the one or more corresponding uracils as one or more corresponding thymidines. In some embodiments, the described method further comprises subjecting the plurality of nucleic acids to one or more enzymatic conversion treatment, prior to the step of obtaining the test dataset, thereby causing one or more methylated cytosines in the plurality of nucleic acids to be converted to one or more corresponding uracils, and the targeted sequencing of the plurality of nucleic acids reads out the one or more corresponding uracils as one or more corresponding thymidines.

In some embodiments, a probe in the plurality of probes includes a respective nucleic acid sequence that is complementary or substantially complementary to the reference genome, or a portion thereof, as represented by a bin in the first plurality of bins, with the exception that the probe includes an adenosine to complement a thymidine in the one or more corresponding thymidines.

In some embodiments, a disease condition in the set of disease conditions exhibits a methylation pattern in which methylation of a first cytosine but not a second cytosine in the genome of the species is characteristic of the disease condition, and absence of methylation of both the first cytosine and the second cytosine is characteristic of an absence of the disease condition. In such embodiments, the method in accordance with some aspects of the present disclosure comprises, prior to the step of obtaining the test dataset, subjecting the plurality of nucleic acids to a bisulfite conversion, thereby causing a plurality of unmethylated cytosines in the plurality of nucleic acids to be converted to a plurality of corresponding uracils. A probe in the plurality of probes may include a respective nucleic acid sequence that is complementary or substantially complementary to the reference genome, or a portion thereof, that includes the first cytosine and the second cytosine, the probe including a first guanosine for the first cytosine, and with the exception that the probe further includes an adenosine for the second cytosine thereby causing the targeted sequencing to selectively read for the disease condition over the absence of the disease condition.

Bisulfite conversion can involve converting cytosine to uracil while leaving methylated cytosines-5-methylcytosine (5-mC)-intact. In some DNA, about 95% of cytosines may not be methylated in the DNA, and the resulting DNA fragments may include many uracils which, in the final sequence reads, are represented by thymines. To address this, in some embodiments, enzymatic conversion processes may be used to treat the nucleic acids prior to sequencing, which can be performed in various ways. An example of a bisulfite-free conversion is described in Liu et at. that describe a bisulfite-free and base-resolution sequencing method, TET-assisted pyridine borane sequencing (TAPS), for non-destructive and direct detection of 5-methylcytosine and 5 -hydroxymethylcytosine without affecting unmodified cytosines. See, Liu et al., “Bisulfite-free direct detection of 5-methylcytosine and 5-hydroxymethylcytosine at base resolution,” Nat Biotechnol. [doi: 10.1038/s41587-019-0041-2. Regardless of the specific enzymatic conversion approach, the methylated cytosines can be converted.

Accordingly, in some embodiments, a first disease condition in the set of disease conditions is characterized by a first epigenetic cytosine methylation pattern in which a first cytosine methylation pattern at a first genomic locus of the species is characteristic of the first disease condition, and a second cytosine methylation pattern, different from the first cytosine methylation pattern, at the first genomic locus is characteristic of an absence of the first disease condition. In these embodiments, the described techniques involve, prior to the step of obtaining the test dataset (block 402 of FIG. 4A), subjecting the plurality of nucleic acids to an enzymatic treatment, thereby causing a plurality of unmethylated cytosines in the plurality of nucleic acids to be converted to a plurality of corresponding modified bases. A first probe in the plurality of probes may include a respective nucleic acid sequence that is complementary or substantially complementary to the first genomic locus, with the exception that the first probe is complementary to the first genomic locus upon conversion of methylated cytosines of the first methylation pattern by the epigenetic enzymatic treatment, thereby causing the targeted sequencing to selectively read, through the first probe, for the first disease condition over the absence of the first disease condition. In some embodiments, the plurality of corresponding modified bases are a plurality of uracils, and the epigenetic enzymatic treatment comprises (i) exposing the plurality of nucleic acids to a ten-eleven translocation (TET) dioxygenase, and (ii) exposing of the plurality of nucleic acids to a borane based reducing agent after exposure to the TET dioxygenase.

Further, in some embodiments, prior to the step of exposing the plurality of nucleic acids to the TET dioxygenase, the plurality of nucleic acids are exposed to β-glucosyltransferase or to KRuO₄. The borane based reducing agent may comprise pyridine borane or 2-picoline borane.

In some embodiments, a second disease condition in the set of disease conditions is characterized by a second epigenetic cytosine methylation pattern in which a third cytosine methylation pattern at a second genomic locus of the species, other than the first genomic locus, is characteristic of the second disease condition; and a fourth cytosine methylation pattern, different from the third cytosine methylation pattern, at the second genomic locus is characteristic of an absence of the second disease condition. A second probe in the plurality of probes can include a respective nucleic acid sequence that is complementary or substantially complementary to the second genomic locus, with the exception that the second probe is complementary to the second genomic locus upon conversion of methylated cytosines of the third methylation pattern by the epigenetic enzymatic treatment, thereby causing the targeted sequencing to selectively read, through the second probe, for the second disease condition over the absence of the second disease condition.

### Blocks 442-448.

In some embodiments, as mentioned above, the plurality of copy number values are in the form of dimension reduction values. Referring to FIG. 4D, in some such embodiments, the step of determining the plurality of copy number values in the form of dimension reduction values comprises calculating the plurality of copy number values as a second plurality of dimension reduction values (e.g., second plurality of dimension reduction values 130 of FIG. 1), as shown at block 442.

In some embodiments, each respective dimension reduction value in the second plurality of dimension reduction values is calculated using all or a portion of the first and/or second plurality of bin values that is specified (e.g., in the form of a weighted linear or nonlinear combination of such bin values) by a corresponding dimension reduction component in a second plurality of dimension reduction components.

In some embodiments, the second plurality of dimension reduction components is obtained from subjecting sequence reads, obtained by targeted sequencing of cell-free nucleic acids in each biological sample from each respective healthy subject in a plurality of reference healthy subjects using the plurality of probes, to a second unsupervised dimension reduction algorithm. More particularly, the second plurality of dimension reduction components can be obtained from subjecting corresponding reference pluralities of bin counts, obtained for the first and/or second plurality of bins across a plurality of reference healthy subjects, to an unsupervised dimension reduction algorithm. For each respective healthy subject in the plurality of reference healthy subjects, sequence reads can be obtained by targeted sequencing of cell-free nucleic acids in a biological sample obtained from the respective reference healthy subject using the same plurality of probes described above for the test subject. In some embodiments, the plurality of reference healthy subjects comprises two or more, three or more, five or more, ten or more, 15 or more, 20 or more, 30 or more, 50 or more, 100 or more, 500 or more, or 1000 or more healthy subjects. In some embodiments, the sequence reads are mapped to the first plurality of bins to arrive at bin counts for the first plurality of bins for each of the reference healthy subjects. In some embodiments, the sequence reads are also mapped to the second plurality of bins to arrive at bin counts for the second plurality of bins for each of the reference healthy subjects. In some embodiments, such bin counts represent unique nucleic fragments that map to the bins. Each reference subject in the plurality of reference subjects therefore can have a corresponding first and/or second plurality of reference bin values. The corresponding first and/or second plurality of reference bin values for each reference healthy subject in the plurality of reference healthy subjects can be subjected to the second unsupervised dimension reduction algorithm in order to arrive at the second plurality of dimension reduction components.

Thus, in embodiments in which each respective dimension reduction value in the second plurality of dimension reduction values is calculated using a weighted linear or non-linear combination of all or a portion of the first and/or second plurality of bin values that is specified by a corresponding dimension reduction component in the second plurality of dimension reduction components, consider the case in which a first dimension reduction value is calculated using a corresponding dimension reduction component in the second plurality of dimension reduction components. Further consider an embodiment in which the first dimension reduction component has the linear form

$${\sum }_{i = 1}^{n} {w}_{i} {x}_{i} ,$$

where i is a positive integer in the set {1, . . . , n}, n is the number of bins in the combination of the first and/or second plurality of bins, each wᵢ is a weight specified by the first dimension reduction component and each xᵢ is the bin value for the i^{th} bin. Here, the weights wᵢ, . . . , wₙ can be determined by unsupervised dimension reduction (second unsupervised dimension reduction algorithm) of the bin values across the plurality of reference healthy subjects whereas the values xᵢ are the bin values of the test subject. Moreover, some of the weights may be zero meaning that not all bin values for the first and/or second plurality of bins contribute to the value of the first dimension reduction component. In some embodiments, the second plurality of dimension reduction components comprises 10 or more, twenty or more, thirty or more, forty or more, fifty or more, 75 or more or 100 or more dimension reduction components.

As shown at block 444, in some embodiments, the second unsupervised dimension reduction algorithm may be a principal component analysis algorithm, a random projection algorithm, an independent component analysis algorithm, or any feature selection method. The feature selection method can be, for example, a sequential backward selection algorithm (block 446). In some embodiments, the second unsupervised dimension reduction algorithm is a principal component analysis (PCA) algorithm, and the second plurality of dimension reduction components is between five and five hundred dimension reduction components (block 448).

PCA can reduce the dimensionality of the bin values by transforming them into a new set of variables (principal components, second plurality of dimension reduction components) that summarize the features of the training set. Principal components (PCs), the form of dimension reduction components obtained using PCA, can be uncorrelated and can be ordered such that the k^{th} PC has the k^{th} largest variance among PCs. The k^{th} PC can be interpreted as the direction that maximizes the variation of the projections of the data points such that it is orthogonal to the first k−1 PCs. The first few PCs can capture most of the variation in the bin values across the plurality of reference healthy subjects. The last few PCs can capture the residual ‘noise’ across the plurality of reference healthy subjects. For further information on principal component analysis and other suitable dimension reduction techniques, see, for example, Fodor, 2002, “A survey of dimension reduction techniques,” Center for Applied Scientific Computing, Lawrence Livermore National, Technical Report UCRL-ID-148494; Cunningham, 2007, “Dimension Reduction,” University College Dublin, Technical Report UCD-CSI-2007-7, Zahorian et al., 2011, “Nonlinear Dimensionality Reduction Methods for Use with Automatic Speech Recognition,” Speech Technologies. doi: 10.5772/16863. ISBN 978-953-307-996-7; and Lakshmi et al., 2016, “2016 IEEE 6th International Conference on Advanced Computing (IACC),” pp. 31-34. doi: 10.1109/IACC.2016.16, ISBN 978-1-4673-8286-1, each of which is hereby incorporated by reference.

Random projection algorithms can be based on the Johnson-Lindenstrauss lemma which states that if points in a vector space are of sufficiently high dimension, then they may be projected into a suitable lower-dimensional space in a way which approximately preserves the distances between the points. In random projection, the original d-dimensional data (the plurality of bin values for each reference healthy subject in the plurality of reference healthy subjects) can be projected to a k-dimensional (k<<d), subspace, using a random k×d—dimensional matrix R whose columns have unit lengths. Here, k can be 10 or more, twenty or more, thirty or more, forty or more, fifty or more, 75 or more or 100 while d is the number of bin values in the first plurality of bin values. Using matrix notation, if X_{d×N} is the original set of N d-dimensional observations, then

$${X}_{k \times N}^{R P} = {R}_{k \times d} {X}_{d \times N}$$

can be the projection of the data onto a lower k-dimensional subspace. Random projection can involve forming the random matrix “R” and projecting the d×N data matrix×onto K dimensions of order O(dkN). In some embodiments, the matrix “R” is generated using a Gaussian distribution. In such embodiments, the first row is a random unit vector uniformly chosen from S^{d-1}. The second row can be a random unit vector from the space orthogonal to the first row, the third row can be a random unit vector from the space orthogonal to the first two rows, and so on. In this way of choosing R, R can be an orthogonal matrix (the inverse of its transpose), and the following properties can be satisfied (i) (spherical symmetry) for any orthogonal matrix A∈O(d), RA and R have the same distribution, (ii) (orthogonality) the rows of R are orthogonal to each other, and (iii) (normality) the rows of R are unit-length vectors. In some embodiments, the Gaussian distribution is replaced with other simpler forms of distribution.

Independent component analysis (ICA) algorithms can include computational methods for separating a multivariate signal into additive subcomponents. This can assume that the subcomponents are non-Gaussian signals (e.g., variations in the first plurality of bin values across the plurality of reference healthy subjects) and that they are statistically independent from each other. ICA can find the independent components (also called factors, latent variables or sources) by maximizing the statistical independence of the estimated components. Many different ways can be used to define a proxy for independence, and this choice can govern the form of the ICA algorithm. Definitions of independence for ICA can include (i) minimization of mutual information (MMI) and (ii) maximization of non-Gaussianity. The MMI family of ICA algorithms can use measures like Kullback-Leibler Divergence and maximum entropy. The non-Gaussianity family of ICA algorithms, motivated by the central limit theorem, can use kurtosis and negentropy. Algorithms for ICA can use centering (subtract the mean to create a zero mean signal), and whitening (usually with the eigenvalue decomposition), and dimensionality reduction (e.g. PCA) as preprocessing steps in order to simplify and reduce the complexity of the problem for the actual iterative algorithm. Whitening and dimension reduction can be achieved with principal component analysis or singular value decomposition. Whitening can ensure that all dimensions are treated equally a priori before the algorithm is run. Well-known algorithms for ICA include infomax, FastICA, JADE, and kernel-independent component analysis, among others.

In some embodiments, the dimension reduction algorithm is a feature selection algorithm. In such embodiments, a corresponding first and/or second plurality of bin values from both subjects with cancer and subjects without cancer are typically used for the training population. That is, the bin values are, for example, regressed against the status (e.g., cancer, no cancer, estimated tumor fraction, etc.) of each training subject. In some embodiments, the feature selection method comprises regularization (e.g., is Lasso, least-angle-regression, or Elastic net) for the first plurality of bin values across the plurality of reference subjects. In some embodiments, the feature selection method comprises application of a decision tree to the first plurality of bin values across the training population. Tree-based methods can partition the feature space into a set of rectangles, and then fit a model (like a constant) in each one. In some embodiments, the decision tree is random forest regression. One specific algorithm that can be used in the present disclosure is a classification and regression tree (CART). Other specific decision tree algorithms can include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. The aim of a decision tree can be to induce a classifier (a tree) from real-world example data. This tree can be used to classify unseen entities that have not been used to derive the decision tree. As such, a decision tree can be derived from the training set (the first bin values across the training population). As discussed above, the training set can contain data for a plurality of reference subjects) the training population). For each respective reference training subject there can be a plurality of first features (bin values) and a class or scalar value for a second feature (cancer, cancer-free, tumor burden, cancer stage) that represents the class of the reference subject.

Another feature selection method that can be used in the systems and methods of the present disclosure can be multivariate adaptive regression splines (MARS). MARS can be an adaptive procedure for regression, and can be well suited for the high-dimensional problems addressed by the present disclosure. MARS can be viewed as a generalization of stepwise linear regression or a modification of the CART method to improve the performance of CART in the regression setting.

In some embodiments, the feature selection method comprises application of Gaussian process regression to the training set (the first bin values across the training population) using the N-dimensional feature space and a single second feature, such as a class or scalar value that represents the class of the reference subject (e.g., cancer, cancer-free, tumor burden, cancer stage, etc.).

### Blocks 450-464.

In some embodiments, with reference to block 450 of FIG. 4E, the plurality of copy number values, are inputted into a trained classifier, thereby determining whether the subject has a disease condition in a set of disease conditions. In some embodiments, as discussed above, the plurality of copy number values are in the form of a second plurality of dimension reduction values.

In some embodiments, the step of determining whether the subject has a disease condition deems the subject to have a particular disease condition in the set of disease conditions. In some embodiments, the described approach may determine that the subject has more than one disease or condition (e.g., two, three, or more than three), and each of the diseases or conditions may be predicted with a probability. The subject may be deemed to have the particular disease condition when the trained classifier predicts the particular disease condition with a higher probability than all other disease conditions in the set of disease conditions. Furthermore, in some embodiments, the set of disease conditions includes a first disease condition that is absence of disease, as shown at block 452 of FIG. 4E.

In some embodiments, as shown at block 454, the step of determining the plurality of copy number values further comprises extracting a plurality of features from the first and/or second plurality of bin values using a feature extraction method. The features can be selected in various ways and they can be based on a type of elements forming the bin values such as copy number values. For example, the features can be based on a length of fragments assigned to a bin, a number of fragments with their terminal ends assigned to a bin, endpoint based copy number determination, allelic imbalance, etc.

The inputting the at least the plurality of copy number values into a trained classifier, in such embodiments, further comprises applying the plurality of features, in addition to the plurality of copy number values, to the trained classifier to determine whether the subject has the disease condition in the set of disease conditions.

The trained classifier used to predict a subject's condition can be a classifier of any suitable type. For example, as shown at block 456, in some embodiments the trained classifier is a neural network algorithm (e.g., a convolutional neural network), a support vector machine algorithm, a Naive Bayes algorithm, a nearest neighbor algorithm, a boosted trees algorithm, a random forest algorithm, a decision tree algorithm, a multi-category logistic regression algorithm, a linear model, or a linear regression algorithm. In some embodiments, the trained classifier is trained using on-target bin values and off-targets bin values obtained from targeted panel sequencing of a plurality of samples (block 458). In some embodiments, the on-target (first plurality) bin values or the off-target (second plurality) bin values across a training population, together with the disease condition of each subject in the training population, are used for training the classifier.

In the described embodiments, the biological sample comprises blood, whole blood, plasma, serum, urine, cerebrospinal fluid, fecal, saliva, sweat, tears, pleural fluid, pericardial fluid, or peritoneal fluid of the subject (block 460). For example, in some embodiments, the biological sample is a blood sample.

The disease condition can be of any type. In some embodiments, as shown at block 462, the set of disease conditions is a set of cancer conditions and the determined disease condition is a cancer condition. In some embodiments, the determined cancer condition is breast cancer, lung cancer, prostate cancer, colorectal cancer, renal cancer, uterine cancer, pancreatic cancer, cancer of the esophagus, lymphoma, head/neck cancer, ovarian cancer, hepatobiliary cancer, melanoma, cervical cancer, multiple myeloma, leukemia, thyroid cancer, bladder cancer, gastric cancer, or a combination thereof. Also, the determined cancer condition can be a predetermined stage of a breast cancer, a lung cancer, a prostate cancer, a colorectal cancer, a renal cancer, a uterine cancer, a pancreatic cancer, a cancer of the esophagus, a lymphoma, a head/neck cancer, an ovarian cancer, a hepatobiliary cancer, a melanoma, a cervical cancer, a multiple myeloma, a leukemia, a thyroid cancer, a bladder cancer, or a gastric cancer.

In some embodiments, the disease condition is clonal hematopoiesis (block 464). The clonal hematopoiesis can be defined as a condition when hematopoietic stem cells (HSCs) or other early blood cell progenitors contribute to the formation of a genetically distinct subpopulation of blood cells. A driver of a clonal population can be thought to be somatic mutations. For example, a clonal population may occur when a stem or progenitor cell acquires one or more somatic mutations that give it a competitive advantage in hematopoiesis over the stem/progenitor cells without these mutations.

As discussed above, the first plurality of bins and the second plurality of bins can represent different portions of a reference genome. For example, in some embodiments, each region of the reference genome that corresponds to a respective bin in the second plurality of bins is different from each region of the reference genome that corresponds to a respective bin in the first plurality of bins. In some embodiments, each region of the reference genome that corresponds to a respective bin in the second plurality of bins comprises an off-target region. As mentioned above, sequence reads corresponding to off-target regions can be acquired as a result of accidental sequencing, and these genomic regions cannot be defined by probes.

In some embodiments, the corresponding region of each respective bin in the first plurality of bins is an on-target region in a plurality of on-target regions, and the off-target region is defined as a region of the reference genome that does not overlap with an on-target region in the plurality of on-target regions.

In various embodiments in accordance with the present disclosure, the bins can have various sizes. For example, in some embodiments, each bin in the second plurality of bins has a size between about 10,000 base pairs and about 250,000 base pairs. In some embodiments, each bin in the second plurality of bins has a size selected from the group consisting of between about 10,000 and about 500,000 nt, between about 50,000 and about 250,000 nt, and between about 100,000 and about 150,000 nt.

In some embodiments, each bin in the second plurality of bins may have the same length. Further, in some embodiments, each bin in the first plurality of bins has a first length, each bin in the first plurality of bins has a second length, the first length is other than the second length, the first length is between about 100 base pairs and about 250,000 base pairs, and the second length is between about 10,000 base pairs and about 250,000 base pairs. In some embodiments, each bin in the first plurality of bins and the second plurality of bins has the same or different length.

In some embodiments, as shown in FIG. 3, each bin in the first plurality of bins is flanked by a respective pair of buffer regions. Each respective pair of buffer regions can be excluded from the second portion of the reference genome collectively represented by the second plurality of bins. Each buffer region in a respective pair of buffer regions can have a length from about 100 base pairs to about 1000 base pairs. For example, in some embodiments, each buffer region in a respective pair of buffer regions has a length of about 200 base pairs.

In some embodiments, the first plurality of bin values and the second plurality of bin values are generated from counts of sequence reads from the targeted sequencing with the plurality of probes. In such embodiments, sequence reads for the second plurality of bin values can be sequenced even though there can be no specific probes for the genomic regions corresponding to the second plurality of probes.

### Training a Classifier.

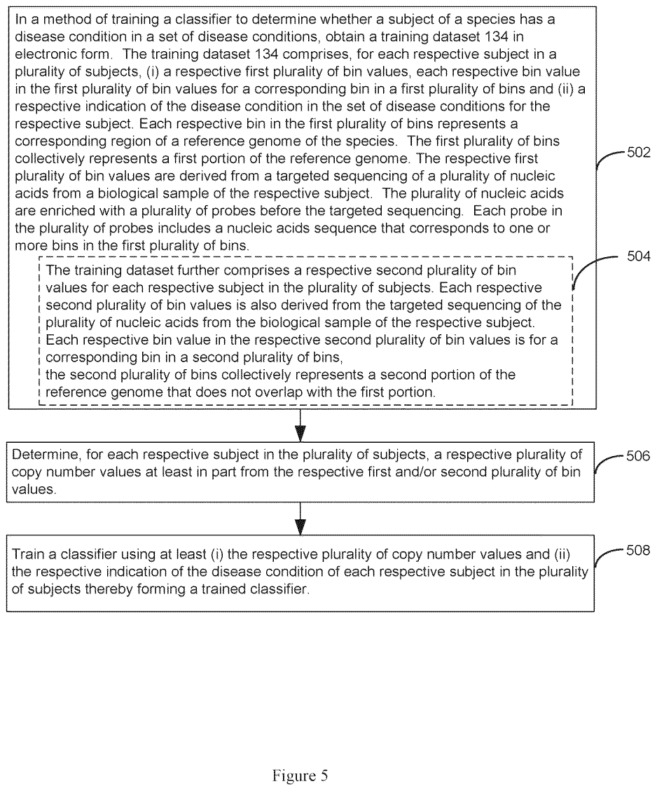

In embodiments discussed above, nucleic acids obtained from a subject are processed to obtain a test dataset that is, in turn, processed to determine copy number values that are inputted into a trained classifier. FIG. 5 illustrates generally a method of training a classifier to determine whether a subject of a species has a disease condition in a set of disease conditions.

Block 502. As shown at block 502 of FIG. 5, the method of training the classifier is provided. The method can be performed in a computer system comprising at least one processor and a memory storing at least one program for execution by the at least one processor, the at least one program comprising instructions for performing the method.

The method can include obtaining a training dataset, in electronic form, that comprises, for each respective subject in a plurality of subjects, (i) a respective first plurality of bin values, each respective bin value in the first plurality of bin values for a corresponding bin in a first plurality of bins and (ii) a respective indication of the disease condition in the set of disease conditions for the respective subject. Each respective bin in the first plurality of bins can represent a corresponding region of a reference genome of the species. The first plurality of bins can collectively represent a first portion of the reference genome. The respective first plurality of bin values can be derived from a targeted sequencing of a plurality of nucleic acids from a biological sample of the respective subject. The plurality of nucleic acids can be enriched using a plurality of probes before the targeted sequencing. Each probe in the plurality of probes can include a nucleic acid sequence that corresponds to one or more bins in the first plurality of bins. A probe may align or substantially align to one or more bins in the first plurality of bins. In some embodiments, the targeted sequencing comprises targeted DNA methylation sequencing.

As described above, the targeted sequencing can be targeted DNA methylation sequencing, which may detect one or more 5-methylcytosine (5mC) and/or 5-hydroxymethylcytosine (5hmC) in the plurality of nucleic acids. In some embodiments, the targeted DNA methylation sequencing comprises bisulfite conversion or enzymatic conversion of one or more unmethylated cytosines, in the plurality of nucleic acids, to a corresponding one or more uracils. The DNA methylation sequencing may read out the one or more uracils as one or more corresponding thymines, and the DNA methylation sequencing may read out the one or more 5mC or 5hmC as one or more corresponding cytosines.

Block 504. In some embodiments, with reference to block 504, the training dataset further comprises a respective second plurality of bin values for each respective subject in the plurality of subjects. Each respective second plurality of bin values can also be derived from the targeted sequencing of the plurality of nucleic acids from the biological sample of the respective subject. Each respective bin value in the respective second plurality of bin values can be for a corresponding bin in a second plurality of bins, and the second plurality of bins collectively can represent a second portion of the reference genome that does not overlap with the first portion. In some embodiments, the probes do not align to the bins in the second plurality of bins, and the second plurality of bins thus represent off-target regions of the reference genome. In some instances, one or more bins in the first plurality of bins overlap with one or more bins in the second plurality of bins. However, in some instances, there is no overlap between bins in the first plurality of bins and bins in the second plurality of bins.

In some embodiments, each respective bin value in the first plurality of bin values or the second plurality of bin values of a respective subject is representative of a number of unique cell-free nucleic acid fragments in the biological sample that both (i) align to the portion of the reference genome corresponding to the bin corresponding to the respective bin value and (ii) have a predetermined methylation pattern, and each cell-free nucleic acid fragment in the number of unique cell-free nucleic acid fragments is represented by one or more sequence reads from the respective targeted sequencing with the plurality of probes that contributed to the respective bin value.

Similar to the way in which bin values for a test dataset are normalized, as discussed in detail above, bin values that are processed for creating copy number values for training the classifier can also be normalized prior to determining, for each respective subject in the plurality of subjects, the respective plurality of copy number values. The bin values, which can be obtained for on-target and/or off-target regions (e.g., the first plurality of bin values and the second plurality of bin values), can be normalized using any of the approaches described herein, or any alternative approaches. Accordingly, the normalization may include bin normalization, correction for GC content, and PCA correction. These can be performed in this order or in any other order.

For example, normalization of bin values can involve determining a respective first measure of central tendency across the respective (first and/or second) plurality of bin values of a respective subject; and replacing each respective bin value in the respective plurality of bin values with the respective bin value divided by the respective first measure of central tendency. The first measure of central tendency may be an arithmetic mean, weighted mean, midrange, midhinge, trimean, Winsorized mean, mean, or mode across the first plurality of bin values.

The normalizing can also include the processing as shown, for instance, in connection with blocks 438 and 440 of FIG. 4C. The correction of bin values for CG content and PCA correction may be performed using any of the approaches described herein. For instance, in some embodiments, normalized bin values (which may or may not be corrected for CG content) can be subjected to an unsupervised dimension reduction algorithm, which results in a certain number of dimension reduction components. A top number (e.g., a positive integer between 2 and 50) of the dimension reduction components can then be used to train the classifier. The first unsupervised dimension reduction algorithm can be a principal component analysis algorithm, a random projection algorithm, an independent component analysis algorithm, or a feature selection method.

The first plurality of bin values and/or the second plurality of bin values can be filtered in various ways. For example, bin value associated with at least one of a germline mutation, high variability, or low mappability can be removed.

The bins for the on-target and off-target regions may not overlap, such that each region of the reference genome that corresponds to a respective bin in the second plurality of bins is different from each region of the reference genome that corresponds to a respective bin in the first plurality of bins. However, in some embodiments, there may be an overlap between the bins for the on-target and off-target regions.

The bins for the on-target and off-target regions may have different sizes, and a size of on-target bins may be smaller. For example, each bin in the first plurality of bins may have a size selected from the group consisting of between about 10 and about 1,000 nt, between about 50 and about 500 nt, and between about 100 and about 250 nt. At the same time, each bin in the second plurality of bins can have a size between about 10,000 base pairs and about 250,000 base pairs. The bins among the first plurality of bins and the second plurality of bins may or may not have the same length. In some embodiments, a bin in the first plurality of bins is flanked by a respective pair of buffer regions, and each respective pair of buffer regions is excluded from a second portion of the reference genome collectively represented by the second plurality of bins. Each buffer region in a respective pair of buffer regions may have a length from about 100 base pairs to about 1000 base pairs (e.g., about 200 base pairs, in some embodiments).

Block 506. The method of training the classifier further can comprise determining, for each respective subject in the plurality of subjects, a respective plurality of copy number values at least in part from the respective first and/or second plurality of bin values (block 506).

Block 508. With reference to block 508, the classifier can then be trained using at least (i) the respective plurality of copy number values and (ii) the respective indication of the disease condition of each respective subject in the plurality of subjects thereby forming a trained classifier. In the described embodiments, a bin value for a bin, representing a portion of a reference genome, can be determined in various ways, e.g., based on sequence read counts, fragment lengths, fragment terminal positions, etc.

The classifier can be trained to determine whether a test subject has one or more disease conditions in the set of disease conditions. Furthermore, the set of disease conditions may include a disease condition that is absence of disease.

In some embodiments, the classifier is trained to predict a disease condition such as, for example, a cancer condition (e.g., absence or presence of cancer) and/or a stage of a cancer condition from any of the cancer conditions described herein.

For training the classifier in accordance with embodiments of the present disclosure, each respective bin value in a respective first plurality of bin values of a respective subject can be representative of a respective number of unique cell-free nucleic acid fragments in the respective biological sample that align to the portion of the reference genome represented by the bin corresponding to the respective bin value as determined by the targeted sequencing. Each cell-free nucleic acid fragment in the respective number of unique cell-free nucleic acid fragments may be represented by one or more sequence reads of the targeted sequencing with the plurality of probes that contribute to the respective bin value.

Any of a variety of classifiers may be suitable for use in processing the plurality of copy number values. In particular, supervised learning algorithms can be of particular use as a classifier in the present disclosure. In the context of the present disclosure, supervised learning algorithms can be algorithms that rely on a set of labeled paired training data examples (e.g., sets of copy number values paired with the cancer condition of the subjects corresponding to the sets of copy number values) to infer a relationship between the copy number values and cancer condition. Nonlimiting examples of supervised learning algorithm can include, but are not limited to neural network algorithms (e.g., convolutional neural networks, deep learning algorithms), support vector machine algorithms (SVM), a Naive Bayes algorithms, nearest neighbor algorithms, random forest algorithms, decision tree algorithms, boosted trees algorithms, regression algorithms, logistic regression algorithms, multi-category logistic regression algorithms, and linear discriminant analysis algorithms.

In some embodiments, the classifier is an unsupervised learning algorithm. In the context of the present disclosure, unsupervised learning algorithms can be algorithms used to draw interferences from training data comprising copy number values that are not paired with their cancer condition. One example of an unsupervised learning algorithm is cluster analysis.

In some embodiments, the classifier is a semi-supervised classifier. In the context of the present disclosure, semi-supervised learning algorithms can be algorithms that make use of both labeled and unlabeled data for training (typically using a relatively small amount of labeled data with a large amount of unlabeled data).

Neural Networks. Neural network algorithms, or artificial neural networks (ANNs), and further including convolutional neural network algorithms (deep learning algorithms), are disclosed in Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Neural networks can be machine learning algorithms that may be trained to map an input data set (e.g., copy number values) to an output data set (e.g., cancer condition, etc.), where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data (e.g., copy number values) or the output of nodes in previous layers, and perform a specific operation, e.g., a summation operation. In some embodiments, a connection from an input to a node is associated with a weight (or weighting factor). In some embodiments, the node may sum up the products of all pairs of inputs, xᵢ, and their associated weights. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLu activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) (e.g., a determination of cancer condition) that the ANN computes are consistent with the examples included in the training data set. The parameters may be obtained from a back propagation neural network training process that may or may not be performed using the same computer system hardware as that used for performing the cell-based sensor signal processing methods disclosed herein.

Any of a variety of neural networks may be suitable for use in processing the sensor signals generated by the cell-based sensor devices and systems of the present disclosure. Examples include, but are not limited to, feedforward neural networks, radial basis function networks, recurrent neural networks, convolutional neural networks, and the like. In some embodiments, the disclosed classifier is a pre-trained ANN or deep learning architecture.

In general, the number of nodes used in the input layer of the ANN or DNN may range from about 10 to about 100,000 nodes. In some embodiments, the number of nodes used in the input layer is at least 10, at least 50, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10,000, at least 20,000, at least 30,000, at least 40,000, at least 50,000, at least 60,000, at least 70,000, at least 80,000, at least 90,000, or at least 100,000. In some embodiments, the number of nodes used in the input layer may be at most 100,000, at most 90,000, at most 80,000, at most 70,000, at most 60,000, at most 50,000, at most 40,000, at most 30,000, at most 20,000, at most 10,000, at most 9000, at most 8000, at most 7000, at most 6000, at most 5000, at most 4000, at most 3000, at most 2000, at most 1000, at most 900, at most 800, at most 700, at most 600, at most 500, at most 400, at most 300, at most 200, at most 100, at most 50, or at most 10. The number of nodes used in the input layer may have any value within this range, for example, about 512 nodes.

In some embodiments, the total number of layers used in the ANN or DNN (including input and output layers) ranges from about 3 to about 20. In some embodiments, the total number of layers is at least 3, at least 4, at least 5, at least 10, at least 15, or at least 20. In some embodiments, the total number of layers is at most 20, at most 15, at most 10, at most 5, at most 4, or at most 3. The total number of layers used in the ANN may have any value within this range, for example, 8 layers.

In some embodiments, the total number of learnable or trainable parameters, e.g., weighting factors, biases, or threshold values, used in the ANN or DNN ranges from about 1 to about 10,000. In some embodiments, the total number of learnable parameters is at least 1, at least 10, at least 100, at least 500, at least 1,000, at least 2,000, at least 3,000, at least 4,000, at least 5,000, at least 6,000, at least 7,000, at least 8,000, at least 9,000, or at least 10,000. Alternatively, the total number of learnable parameters is any number less than 100, any number between 100 and 10,000, or a number greater than 10,000. In some embodiments, the total number of learnable parameters is at most 10,000, at most 9,000, at most 8,000, at most 7,000, at most 6,000, at most 5,000, at most 4,000, at most 3,000, at most 2,000, at most 1,000, at most 500, at most 100 at most 10, or at most 1. The total number of learnable parameters used may have any value within this range, for example, about 2,200 parameters.

SVMs. SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs can separate a given set of binary labeled data training set (e.g., the copy number values provided with a binary label of either possessing or not possessing cancer) with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space.

Naïve Bayes algorithms. Naïve Bayes classifiers can be a family of “probabilistic classifiers” based on applying Bayes' theorem with strong (naïve) independence assumptions between the features. In some embodiments, they are coupled with Kernel density estimation. See, Hastie, Trevor, 2001, The elements of statistical learning: data mining, inference, and prediction, Tibshirani, Robert, Friedman, J. H. (Jerome H.), New York: Springer, which is hereby incorporated by reference.

Nearest neighbor algorithms. Nearest neighbor classifiers can be memory-based and include no classifier to be fit. Given a query point x₀, the k training points x₍ᵣ₎, r, . . . , k closest in distance to x₀ are identified and then the point x₀ can be classified using the k nearest neighbors. Ties can be broken at random. In some embodiments, Euclidean distance in feature space is used to determine distance as:

$${d}_{( i )} = ‖ {x}_{( i )} - {x}_{( 0 )} ‖$$

In some embodiments, when the nearest neighbor algorithm is used, the bin values for the training set are standardized to have mean zero and variance 1. In some embodiments, the nearest neighbor analysis is refined to address issues of unequal class priors, differential misclassification costs, and feature selection. Many of these refinements can involve some form of weighted voting for the neighbors. For more disclosure on nearest neighbor analysis, see Duda, Pattern Classification, Second Edition, 2001, John Wiley & Sons, Inc.; and Hastie, 2001, The Elements of Statistical Learning, Springer, New York, each of which is hereby incorporated by reference in its entirety.

Random forest, Decision Tree, and boosted tree algorithms. Decision trees are described generally by Duda, 2001, Pattern Classification, John Wiley & Sons, Inc., New York, pp. 395-396, which is hereby incorporated by reference. Tree-based methods can partition the feature space into a set of rectangles, and then fit a model (like a constant) in each one. In some embodiments, the decision tree is random forest regression. One specific algorithm that can be used is a classification and regression tree (CART). Other specific decision tree algorithms can include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

Regression, logistic regression, and multi-category logistic regression. The regression algorithm can be any type of regression. For example, in some embodiments, the regression algorithm is logistic regression. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the regression algorithm is logistic regression with lasso, L2 or elastic net regularization. In some embodiments, those extracted features (copy number values) that have a corresponding regression coefficient that fails to satisfy a threshold value are pruned (removed from) the plurality of copy number vale. In some embodiments, this threshold value is zero. Thus, in such embodiments, those copy number values that have a corresponding regression coefficient that is zero from the above-described regression are not considered by the classifier. In some embodiments, for instance, in which L2 regularization is employed, the threshold value is 0.1. Thus, in such embodiments, those copy number values that have a corresponding regression coefficient whose absolute value is less than 0.1 from the above-described regression are removed from the plurality of copy number values and are not considered by the classifier. In some embodiments, the threshold value is a value between 0.1 and 0.3. An example of such embodiments is the case where the threshold value is 0.2. In such embodiments, those copy number values that have a corresponding regression coefficient whose absolute value is less than 0.2 from the above-described regression are not considered by the classifier. In some embodiments, a generalization of the logistic regression model that handles multicategory responses serves as the classifier. A number of such multi-category logit models described in Agresti, An Introduction to Categorical Data Analysis, 1996, John Wiley & Sons, Inc., New York, Chapter 8, hereby incorporated by reference in its entirety.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the classifier (linear classifier) in some embodiments of the present disclosure.

Ensembles of classifiers and boosting. In some embodiments, an ensemble (two or more) of classifiers is used. In some embodiments, a boosting technique such as AdaBoost is used in conjunction with many other types of learning algorithms to improve the performance of the classifier. In this approach, the output of any of the classifiers disclosed herein, or their equivalents, is combined into a weighted sum that represents the final output of the boosted classifier.

Clustering algorithm. In some embodiments, the classifier is a clustering applied to the plurality of copy number values In such embodiments, the inputting the plurality of copy number values into the model comprises determining whether the plurality of copy number values of the test subject co-clusters with the plurality of copy number values from a training set. In some such embodiments, this clustering comprises unsupervised clustering. To illustrate how the plurality of copy number values are used in clustering, consider the case in which ten copy number values are used. In some embodiments, each reference subject of a training set can have values for each of the ten copy number values. In some embodiments, each reference subject of the training set has measurement values for some of the ten copy number values and the missing values are either filled in using imputation techniques or ignored (marginalized). In some embodiments, each subject of the training set has values for some of the ten copy number values and the missing values are filled in using constraints. The values from a reference subject in the training set define the vector: X₁, X₂, X₃, X₄, X₅, X₆, X₇, X₈, X₉, X₁₀ where Xᵢ can be the value of the i^{th} copy number value for a particular reference subject. If there are Q reference subject in the training set, selection of the 10 copy number values can define Q vectors. Note that, as discussed above, the systems and methods of the present disclosure cannot include that each copy number value used in the vectors be represented in every single vector Q. In some embodiments, data from a reference subject in which one of the i^{th} copy number values has not been determined can still be used for clustering by assigning the missing copy number value a value of either “zero” or some other normalized value. In some embodiments, prior to clustering, the copy number value in the vectors are normalized to have a mean value of zero (or some other predetermined mean value) and unit variance (or some other predetermined variance value). Those members of the training set that exhibit similar measurement patterns across their respective vectors can tend to cluster together. A particular combination of set of copy number values can be considered to be a good classifier in this aspect of the present disclosure when the vectors cluster into identifiable groups found in the training set with respect to a target feature (e.g., cancer, absence of cancer, stage of cancer, etc.). For instance, if the training set includes class a: reference subjects that have cancer, and class 2: reference subjects that do not have cancer, an ideal clustering model can cluster the training set and, in fact, the test subject, into two groups, with one cluster group uniquely representing class 1 and the other cluster group uniquely representing class 2.

The clustering can find natural groupings in a dataset. To identify natural groupings, two issues can be addressed. First, a way to measure similarity (or dissimilarity) between two samples can be determined. This metric (similarity measure) can be used to ensure that the samples in one cluster are more like one another than they are to samples in other clusters. Second, a mechanism for partitioning the data into clusters using the similarity measure can be determined.

One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. In some embodiments, clustering cannot include the use of a distance metric. For example, a nonmetric similarity function s(x, x′) can be used to compare two vectors x and x′. s(x, x′) can be a symmetric function whose value is large when x and x′ are somehow “similar.”

Once a method for measuring “similarity” or “dissimilarity” between points in a dataset has been selected, clustering can include a criterion function that measures the clustering quality of any partition of the data. Partitions of the data set that extremize the criterion function can be used to cluster the data.

Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. Such clustering can be on the set of first features {p₁, . . . , p_{N-K}} (or the principal components derived from the set of first features). In some embodiments, the clustering comprises unsupervised clustering (block 490) where no preconceived notion of what clusters can form when the training set is clustered are imposed.

Using cross-validation to train a classifier. In some embodiments, k-fold cross-validation is used to train a classifier. When a specific value for k is chosen, it may be used in place of k in the reference to the model, such as k=10 becoming 10-fold cross-validation. Cross-validation can be used in applied machine learning to estimate a machine learning model on unseen data. Cross-validation can use a limited sample in order to estimate how the model is expected to perform in general when used to make predictions on data not used during the training of the model. The process of k-fold cross-validation can comprise:
(i) shuffling the training dataset randomly;
(ii) splitting the training dataset into k groups; and
(iii) For each unique group (for each of the k groups):
   Taking the group as a hold out or test data set
   Taking the remaining groups as a training data set
   Fitting a model on the training set and evaluate it on the test set
   Retaining the evaluation score and discard the model
   Summarizing the characteristics of the model (e.g. sensitivity, specificity, etc.) using the sample of model evaluation scores.

Each observation in the data sample (each subject in the training set) can be assigned to an individual group and stay in that group for the duration of the procedure. Each person in the training set can be given the opportunity to be used in the hold out set 1 time and used to train the model k−1 times.

Optional feature extraction. In some embodiments, the step of determining, for each respective subject in the plurality of subjects, a respective plurality of copy number values (block 506), further comprises extracting a plurality of features from the respective first plurality of bin values using a feature extraction method. In such embodiments, the training the classifier (block 508) further comprises using the plurality of features, in addition to the respective plurality of copy number values and the respective indication of the disease condition, to train the classifier.

The feature extraction method can involve any suitable technique. For example, in some embodiments, the feature extraction method may be a dimension reduction algorithm such as, e.g., a principal component analysis algorithm, a factor analysis algorithm, Sammon mapping, curvilinear components analysis, a stochastic neighbor embedding (SNE) algorithm, an Isomap algorithm, a maximum variance unfolding algorithm, a locally linear embedding algorithm, a t-SNE algorithm, a non-negative matrix factorization algorithm, a kernel principal component analysis algorithm, a graph-based kernel principal component analysis algorithm, a linear discriminant analysis algorithm, a generalized discriminant analysis algorithm, a uniform manifold approximation and projection (UMAP) algorithm, a LargeVis algorithm, a Laplacian Eigenmap algorithm, or a Fisher's linear discriminant analysis algorithm. See, for example, Fodor, 2002, “A survey of dimension reduction techniques,” Center for Applied Scientific Computing, Lawrence Livermore National, Technical Report UCRL-ID-148494; Cunningham, 2007, “Dimension Reduction,” University College Dublin, Technical Report UCD-CSI-2007-7, Zahorian et al., 2011, “Nonlinear Dimensionality Reduction Methods for Use with Automatic Speech Recognition,” Speech Technologies. doi: 10.5772/16863. ISBN 978-953-307-996-7; and Lakshmi et al., 2016, “2016 IEEE 6th International Conference on Advanced Computing (IACC),” pp. 31-34. doi: 10.1109/IACC.2016.16, ISBN 978-1-4673-8286-1, each of which is hereby incorporated by reference. Accordingly, in some embodiments, the dimension reduction algorithm is a principal component analysis (PCA) algorithm, and each respective extracted feature comprises a respective principal component derived by the PCA. In such embodiments, the corresponding subset of the first plurality of extracted features can be limited to a threshold number of principal components calculated by the PCA algorithm. The threshold number of principal components can be, for example, 5, 10, 20, 50, 100, 1000, 1500, or any other number. In some embodiments, each principal component calculated by the PCA algorithm is assigned an eigenvalue by the PCA algorithm, and the corresponding subset of the first plurality of extracted features is limited to the threshold number of principal components assigned the highest eigenvalues.

### Select Human Genomic Regions Used for the First Plurality (On-Target) Bins.

In various embodiments, the selected target genomic regions used for the first plurality (on-target) bins can be located in various positions in a genome, including but not limited to exons, introns, intergenic regions, and other parts. In some embodiments, probes targeting non-human genomic regions, such as those targeting viral genomic regions, can be added.

In some embodiments of the present disclosure, each bin in the first plurality of bins is drawn from a panel of genomic regions that is designed for targeted selection of cancer-specific methylation patterns. In some embodiments, each such genomic region is drawn from Table 2 of International Patent Application No. PCT/US2020/015082, entitled “Detecting Cancer, Cancer Tissue or Origin, or Cancer Type,” filed Jan. 24, 2020, which is hereby incorporated by reference, including the Sequence Listing referenced therein.

SEQ ID NOs 452,706-483,478 of PCT/US2020/015082 provide further information about certain hypermethylated or hypomethylated target genomic regions. These SEQ ID NO. records identify target genomic regions that can be differentially methylated in samples from specified pairs of cancer types. The target genomic regions of SEQ ID NOs 452,706-483,478 of PCT/US2020/015082 are drawn from list 6 of PCT/US2020/015082. Many of the same target genomic regions are also found in lists 1-5 and 7-16 of PCT/US2020/015082. The entry for each SEQ ID can indicate the chromosomal location of the target genomic region relative to hg19, whether cfDNA fragments to be enriched from the region are hypermethylated or hypomethylated, the sequence of one DNA strand of the target genomic region, and the pair or pairs of cancer types that are differentially methylated in that genomic region. As the methylation status of some target genomic regions distinguish more than one pair of cancer types, each entry can identify a first cancer type as indicated in TABLE 3 of PCT/US2020/015082, including the Sequence Listing referenced therein and one or more second cancer types.

In some embodiments, the first plurality of bins (on-target bins) of the present disclosure includes a separate bin for each of at least 200, 500, 1,000, 5,000, 10,000, 15,000, 20,000, 30,000, 40,000, or 50,000 target genomic regions in any one of lists 1-16, lists 1-3, lists 13-16, list 12, list 4, or lists 8-11 of PCT/US2020/015082. In some embodiments, the first plurality of bins (on-target bins) of the present disclosure includes a separate bin for each of at least 200, 500, 1,000, 5,000, 10,000, 15,000, 20,000, 30,000, 40,000, or 50,000 target genomic regions in any combination of one or more lists 1-16 of PCT/US2020/015082 (e.g., such as lists 1-3, lists 13-16, list 12, list 4, or lists 8-11).

In some embodiments, the first plurality of bins (on-target bins) of the present disclosure includes a separate bin for each of at least 20%, 30%, 40%, 50%, 60%, 70%, 80%, 90%, or 95% of the target genomic regions in any one of lists 1-16 of PCT/US2020/015082. In some embodiments, the first plurality of bins (on-target bins) of the present disclosure includes a separate bin for each of at least 20%, 30%, 40%, 50%, 60%, 70%, 80%, 90% or 95% of the target genomic regions in any combination of one or more lists 1-16 of PCT/US2020/015082 (e.g., such as lists 1-3, lists 13-16, list 12, list 4, or lists 8-11).

### Additional Select Human Genomic Regions Used for the First Plurality of Bins (On-Target Bins).

In some embodiments of the present disclosure, each bin in the first plurality of bins (on-target bins) is drawn from a panel of genomic regions that is designed for targeted selection of cancer-specific methylation patterns. In some embodiments, each such genomic region is drawn from Table 2 of International Patent Application No. PCT/US2019/053509, published as WO2020/669350A1, entitled “Methylated Markers and Targeted Methylation Probe Panel,” filed Sep. 27, 2019, which is hereby incorporated by reference, including the Sequence Listing referenced therein.

The sequence listing of WO2020/669350A1 includes the following information: (1) SEQ ID NO, (2) a sequence identifier that identifies (a) a chromosome or contig on which the CpG site is located and (b) a start and stop position of the region, (3) the sequence corresponding to (2) and (4) whether the region was included based on its hypermethylation or hypomethylation score. The chromosome numbers and the start and stop positions are provided relative to a known human reference genome, GRCh37/hgl9. The sequence of GRCh37/hgl9 is available from the National Center for Biotechnology Information (NCBI), the Genome Reference Consortium, and the Genome Browser provided by Santa Cruz Genomics Institute.

Generally, a bin in the first plurality of bins (on-target bins) can encompass any of the CpG sites included within the start/stop ranges of any of the targeted regions included in lists 1-8 of WO2020/069350.

In some embodiments, the first plurality of bins (on-target bins) of the present disclosure includes a separate bin for each of at least 200, 500, 1,000, 5,000, 10,000, 15,000, 20,000, 30,000, 40,000, or 50,000 target genomic regions in any one of lists 1-8 of WO2020/069350. In some embodiments, the first plurality of bins (on-target bins) of the present disclosure includes a separate bin for each of at least 200, 500, 1,000, 5,000, 10,000, 15,000, 20,000, 30,000, 40,000, or 50,000 target genomic regions in any combination of lists 1-8 of WO2020/069350.

In some embodiments, the first plurality of bins (on-target bins) of the present disclosure includes a separate bin for each of at least 20%, 30%, 40%, 50%, 60%, 70%, 80%, 90%, or 95% of the target genomic regions in any one of lists 1-8 of WO2020/069350. In some embodiments, the first plurality of bins (on-target bins) of the present disclosure includes a separate bin for each of at least 20%, 30%, 40%, 50%, 60%, 70%, 80%, 90% or 95% of the target genomic regions in any combination of lists 1-8 of WO2020/069350.

### Additional Select Human Genomic Regions Used for the First Plurality of Bins (On-Target Bins).

In some embodiments of the present disclosure, each bin in the first plurality of bins (on-target bins) is drawn from a panel of genomic regions that is designed for targeted selection of cancer-specific methylation patterns. In some embodiments, each such bin corresponds to a genomic region in any of Tables 1-24 of International Patent Application No. PCT/US2019/025358, published as WO2019/195268A2, entitled “Methylated Markers and Targeted Methylation Probe Panels,” filed Apr. 2, 2019, which is hereby incorporated by reference.

In some embodiments, each bin in the first plurality of bins (on-target bins) of the present disclosure maps to a genomic region listed in one or more of Table 1, 2, 3, 4, 5, 6, 7, 8, 9, 10, 11, 12, 13, 14, 15, 16, 17, 18, 19, 20, 21, 22, 23 and/or 24 of WO2019/195268A2.

In some embodiments, an entirety of plurality of the bins of the present disclosure together are configured to map to at least 30%, 40%, 50%, 60%, 70%, 80%, 90% or 95% of the genomic regions in one or more of Tables 1-24 of WO2019/195268A2. In some such embodiments, each bin in the plurality of bins maps to a single unique corresponding genomic region in any of Tables 1-24 of WO2019/195268A2. In some such embodiments, a bin in the plurality of bins maps of the present disclosure map to one, two, three, four, five, six, seven, eight, nine or ten unique corresponding genomic region in any combination of Tables 1-24 of WO2019/195268A2.

In some such embodiments, each bin in the plurality of bins (on-target bins) of the present disclosure maps to a single unique corresponding genomic region in any of Tables 2-10 or 16-24 of WO2019/195268A2. In some such embodiments, a bin in the first plurality of bins (on-target bins) maps to one, two, three, four, five, six, seven, eight, nine or ten unique corresponding genomic region in any combination of Tables 2-10 or 16-24 of WO2019/195268A2.

In some embodiments, the first plurality of bins (on-target bins) of the present disclosure together are configured to map to at least 30%, 40%, 50%, 60%, 70%, 80%, 90% or 95% of the genomic regions in Table 1, 2, 3, 4, 5, 6, 7, 8, 9, 10, 11, 12, 13, 14, 15, 16, 17, 18, 19, 20, 21, 22, 23, and/or 24 of WO2019/195268A2.

Protocol for Obtaining Methylation Information from Sequence Reads of Fragments in a Biological Sample.

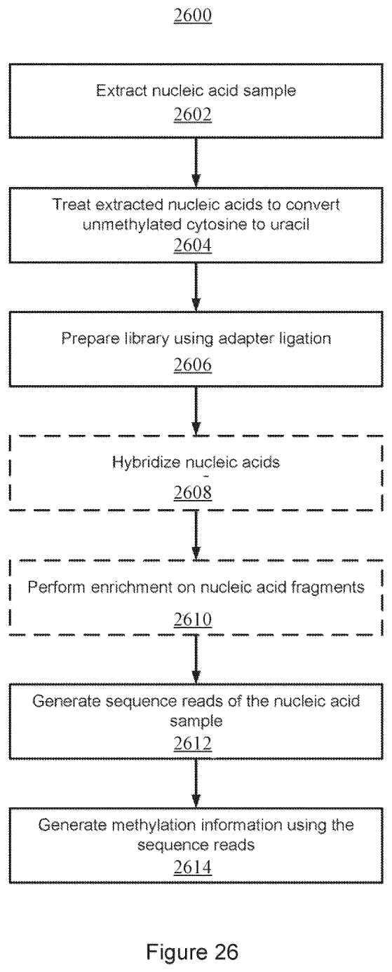

FIG. 26 is a flowchart describing a process 2600 of sequencing fragments (cell-free nucleic acids) and determining methylation states for one or more CpG sites in sequenced fragments, according to some embodiments of the present disclosure. In some embodiments, a methylation state vector is identified for each fragment (cell-free nucleic acid).

In step 2602, nucleic acid (e.g., DNA or RNA) is extracted from a corresponding biological sample of a respective subject. In the present disclosure, DNA and RNA can be used interchangeably unless otherwise indicated. However, the examples described herein can focus on DNA for purposes of clarity and explanation. The biological sample can include nucleic acid molecules derived from any subset of the human genome, including the whole genome. The biological sample can include blood, plasma, serum, urine, fecal, saliva, other types of bodily fluids, or any combination thereof. In some embodiments, methods for drawing a blood sample (e.g., syringe or finger prick) can be less invasive than procedures for obtaining a tissue biopsy obtained via surgery. The extracted sample can comprise cfDNA and/or ctDNA. If a subject has a disease state, such as cancer, cell free nucleic acids (e.g., cfDNA) in an extracted sample from the subject generally includes detectable level of the nucleic acids that can be used to assess a disease state.

In step 2604, the extracted nucleic acids (e.g., including cfDNA fragments) are treated to convert unmethylated cytosines to uracils. In some embodiments, the method 2600 uses a bisulfite treatment of the samples that converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, CA)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, e.g., APOBEC-Seq (NEBiolabs, Ipswich, MA).

In step 2606, a sequencing library is prepared. In some embodiments, the preparation includes at least two steps. In a first step, an ssDNA adapter is added to the 3′-OH end of a bisulfite-converted ssDNA molecule using an ssDNA ligation reaction. In some embodiments, the ssDNA ligation reaction uses CircLigase II (Epicentre) to ligate the ssDNA adapter to the 3′-OH end of a bisulfite-converted ssDNA molecule, where the 5′-end of the adapter is phosphorylated and the bisulfite-converted ssDNA has been dephosphorylated (e.g., the 3′ end has a hydroxyl group). In another embodiment, the ssDNA ligation reaction uses Thermostable 5′ AppDNA/RNA ligase (available from New England BioLabs (Ipswich, MA)) to ligate the ssDNA adapter to the 3′-OH end of a bisulfite-converted ssDNA molecule. In this example, the first UMI adapter is adenylated at the 5′-end and blocked at the 3′-end. In another embodiment, the ssDNA ligation reaction uses a T4 RNA ligase (available from New England BioLabs) to ligate the ssDNA adapter to the 3′-OH end of a bisulfite-converted ssDNA molecule.

In a second step, a second strand DNA can be synthesized in an extension reaction. For example, an extension primer, which hybridizes to a primer sequence included in the ssDNA adapter, is used in a primer extension reaction to form a double-stranded bisulfite-converted DNA molecule. Optionally, in some embodiments, the extension reaction uses an enzyme that is able to read through uracil residues in the bisulfite-converted template strand.

Optionally, in a third step, a dsDNA adapter can be added to the double-stranded bisulfite-converted DNA molecule. Then, the double-stranded bisulfite-converted DNA can be amplified to add sequencing adapters. For example, PCR amplification using a forward primer that includes a P5 sequence and a reverse primer that includes a P7 sequence is used to add P5 and P7 sequences to the bisulfite-converted DNA. Optionally, during library preparation, unique molecular identifiers (UMI) can be added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis.

In an optional step 2608, the nucleic acids (e.g., fragments) can be hybridized. Hybridization probes (also referred to herein as “probes”) may be used to target, and pull down, nucleic acid fragments informative for disease states. For a given workflow, the probes can be designed to anneal (or hybridize) to a target (complementary) strand of DNA or RNA. The target strand can be the “positive” strand (e.g., the strand transcribed into mRNA, and subsequently translated into a protein) or the complementary “negative” strand. The probes can range in length from 10s, 100s, or 1000s of base pairs. Moreover, the probes can cover overlapping portions of a target region.

In an optional step 2610, the hybridized nucleic acid fragments can be captured and enriched, e.g., amplified using PCR. In some embodiments, targeted DNA sequences can be enriched from the library. This is used, for example, where a targeted panel assay is being performed on the samples. For example, the target sequences can be enriched to obtain enriched sequences that can be subsequently sequenced. In general, any method can be used to isolate, and enrich for, probe-hybridized target nucleic acids. For example, a biotin moiety can be added to the 5′-end of the probes (i.e., biotinylated) to facilitate isolation of target nucleic acids hybridized to probes using a streptavidin-coated surface (e.g., streptavidin-coated beads).

In step 2612, sequence reads are generated from the nucleic acid sample, e.g., enriched sequences. Sequencing data can be acquired from the enriched DNA sequences by any method. For example, the method can include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLID sequencing), nanopore sequencing (Oxford Nanopore Technologies), or paired-end sequencing. In some embodiments, massively parallel sequencing is performed using sequencing-by-synthesis with reversible dye terminators.

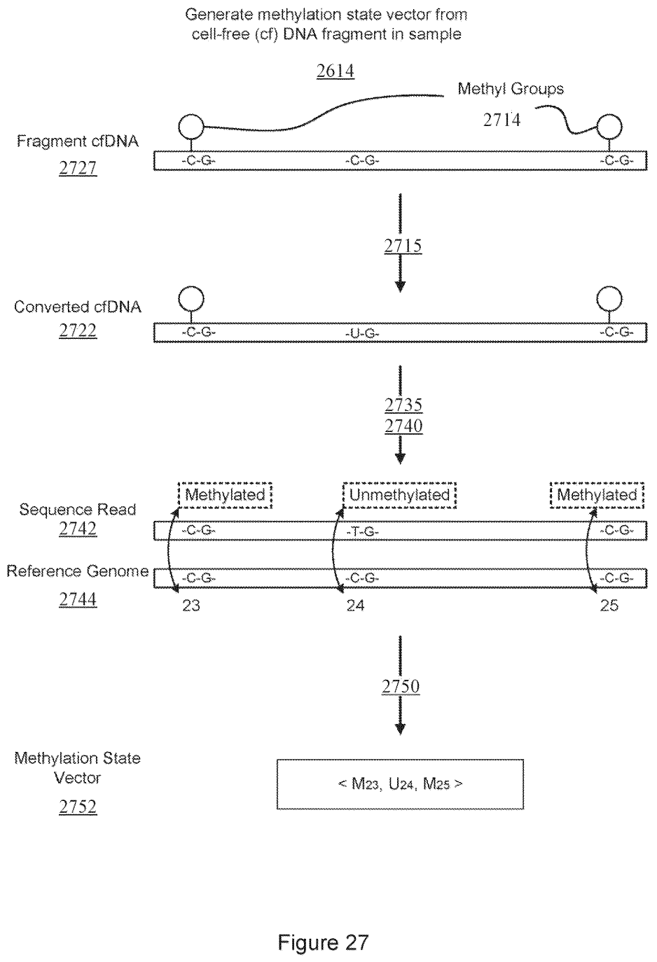

In step 2614, a sequence processor can generate methylation information using the sequence reads. A methylation state vector can then be generated using the methylation information determined from the sequence reads. FIG. 27 is an illustration of the process 2600 of sequencing a cfDNA molecule to obtain a methylation state vector 2752, according to some embodiments of the present disclosure. As an example, a cfDNA fragment is 2712 received that, in this example, contains three CpG sites. As shown, the first and third CpG sites of the cfDNA fragment (molecule) 2712 are methylated 2714. During the treatment step 2715, the cfDNA molecule 2712 is converted to generate a converted cfDNA molecule 2722. During the treatment 2715, the second CpG site, which was unmethylated, has its cytosine converted to uracil. However, during the treatment 2715, the first and third CpG sites were not converted.

After conversion, a sequencing library is prepared 2735 and sequenced 2740, thereby generating a sequence read 2742. The sequence read 2742 is aligned to a reference genome 2744. The reference genome 2744 provides the context as to what position in a human genome the fragment cfDNA originates from. In this simplified example, the analytics system aligns the sequence read 2742 such that the three CpG sites correlate to CpG sites 23, 24, and 25 (arbitrary reference identifiers used for convenience of description). The disclosed systems and methods thus generate information both on methylation status of all CpG sites on the cfDNA fragment (molecule) 2612 and the position in the human genome that the CpG sites map to. As shown, the CpG sites on sequence read 2742, which were methylated, are read as cytosines. In this example, the cytosines appear in the sequence read 2742 in the first and third CpG site, which allows one to infer that the first and third CpG sites in the original cfDNA molecule were methylated. Whereas, the second CpG site is read as a thymine (U is converted to T during the sequencing process), and thus, one can infer that the second CpG site was unmethylated in the original cfDNA molecule. With these two pieces of information, the methylation status and location, the disclosed systems and methods generate a methylation state vector 2752 for the fragment cfDNA 2612. In this example, the resulting methylation state vector 2752 is <M₂₃, U₂₄, M₂₅>, where M corresponds to a methylated CpG site, U corresponds to an unmethylated CpG site, and the subscript number corresponds to a position of each CpG site in the reference genome.

### Example 1—Analysis of CCGA

The inventors conducted experiments demonstrating efficacy of cancer detection using the on-target regions, off-target regions, or a combination of on-target and off-target regions. The experiments were conducted using samples from the Circulating Cell-Free Genome Atlas Study (CCGA) (NCT02889978). The CCGA study was designed for developing a plasma cell-free DNA (cfDNA)-based multi-cancer detection assay. A number of sequencing processes were implemented for the CCGA study. Subjects from the CCGA were used in the present disclosure. CCGA is a prospective, multi-center, observational cfDNA-based early cancer detection study that has enrolled 9,977 of 15,000 demographically-balanced participants at 141 sites. Blood was collected from 1,785 participants-984 participants with newly diagnosed, untreated cancer (20 tumor types, all stages) and 749 participants with no cancer diagnosis (controls) for plasma cfDNA extraction. Three sequencing assays were performed on the blood drawn from each participant: paired cfDNA and white blood cell (WBC) targeted sequencing (507 genes, 60,000×) for single nucleotide variants/indels (the ART sequencing assay), paired cfDNA and WBC whole-genome sequencing (WGS, 30×) for copy number variation, and cfDNA whole-genome bisulfite sequencing (WGBS, 30×) for methylation.

In the experiments conducted by the inventors, bin counts were calculated as a number of unique cfDNA fragments in each bin from a plurality of bins as determined from the ART sequencing assay of subjects in the CCGA study. The training dataset thus comprised the sequence reads obtained using the ART sequencing assay in the CCGA study. The bin counts were subjected to dimensionality reduction using a principal component analysis to generate a number of features (e.g., principal components), and a binary logistic regression classifier was trained in accordance with embodiments of the present disclosure using the generated features.

A 10-fold cross-validation was used. Cross-validation is a resampling procedure used to evaluate machine learning models (classifiers) on a limited data sample. The procedure has a single parameter called k that refers to the number of groups that a given data sample is to be split into. For instance, in this example the data was split into 10 groups. As such, the procedure is often called k-fold cross-validation. When a specific value for k is chosen, it may be used in place of k in the reference to the model, such as k=10 becoming 10-fold cross-validation.

In the disclosed examples, 7323 bins were used for on-target regions (with 200 bp padding), sequence reads, from the ART assay (the paired cfDNA and white blood cell targeted sequencing of 507 genes with 60,000× coverage for nucleotide variants, insertions, or deletions as described above), that fall within the bins were used to determine copy number values, a dataset from a plurality of young healthy reference subjects from the CCGA dataset was used for a baseline correction of the bin values obtained for the on-target regions. The bins for off-target regions were about 100 kb in length, and 25061 bins were defined for the off-target regions. The dataset from the plurality of young healthy reference subjects in the CCGA study was used for a baseline correction of the bin values obtained for the off-target regions.

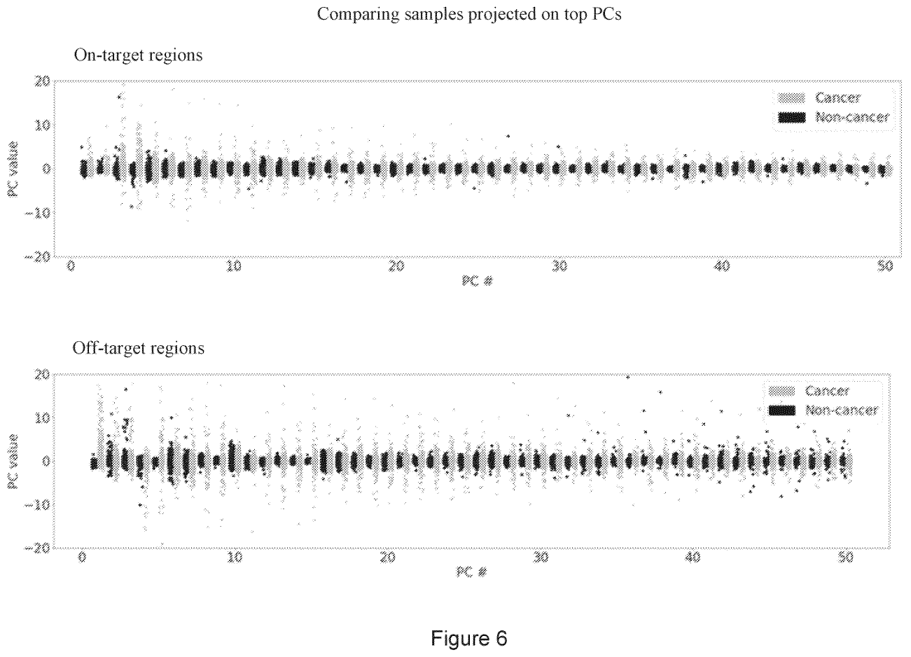

In the results described in FIGS. 6-25, bin values were normalized, subjected for correction for GC content and subjected to PCA normalization. The samples were projected to a certain number of principal components.

For FIG. 6, a first set of dimension reduction components were obtained by subjecting a corresponding first plurality bin values (on target) obtained by targeted sequencing of cell-free nucleic acids in a corresponding biological sample of a respective healthy subject using the plurality of probes, for each reference healthy subject in a plurality of reference healthy subjects, to an unsupervised dimension reduction algorithm. Also, a second set of dimension reduction components were obtained by subjecting a corresponding second plurality bin values (off-target) obtained by targeted sequencing of cell-free nucleic acids in a corresponding biological sample of a respective healthy subject using the plurality of probes, for each reference healthy subject in the plurality of reference healthy subjects, to an unsupervised dimension reduction algorithm.

Each respective dimension reduction component in the first set of dimension reduction components is a weighted combination of all or a portion of the first plurality of bin values that is specified by the respective dimension reduction component.

Each respective dimension reduction component in the second set of dimension reduction components is a weighted combination of all or a portion of the second plurality of bin values that is specified by the respective dimension reduction component.

Thus, to form FIG. 6 upper panel, the first plurality of bin values (on-target) determined for each subject in the CCGA study was individually projected onto the first set of dimension reduction components. Thus, for each subject in the CCGA study, a corresponding dimension reduction component value was computed for each dimension reduction component in the first set of dimension reduction components. These dimension reduction values were then plotted, on a dimension reduction component by dimension reduction component basis, in the upper panel of FIG. 6, with the dimension reduction component values of subjects in the CCGA having cancer plotted together (grey) and the dimension reduction component values of subjects in the CCGA not having cancer plotted together (black). For FIG. 6, the unsupervised dimension reduction algorithm was principal component analysis and the first set of dimension reduction components consisted of 50 principal components.

To form FIG. 6, lower panel, the second plurality of bin values (off-target) determined for each subject in the CCGA study was individually projected onto the second set of dimension reduction components. Thus, for each subject in the CCGA study, a corresponding dimension reduction component value was computed for each dimension reduction component in the second set of dimension reduction components. These dimension reduction values were then plotted, on a dimension reduction component by dimension reduction component basis, in the lower panel of FIG. 6, with the dimension reduction component values of subjects in the CCGA having cancer plotted together (grey) and the dimension reduction component values of subjects in the CCGA not having cancer plotted together (black). For FIG. 6, the second set of dimension reduction components also consisted of 50 principal components.

For FIG. 6, the first set of principal components (upper plot) are arranged from most significant to least significant principal component. Likewise, the second set of principal components (lower plot) are arranged from most significant to least significant principal component. FIG. 6 shows that the overall range of principal component values, across the ranked first and second set of principal components (dimension reduction components) has a similar pattern for the cancer and non-cancer subjects. This indicates that the off-target regions, even though they contained no probes used in the targeted sequencing, nevertheless contain information regarding the disease condition of the subjects.

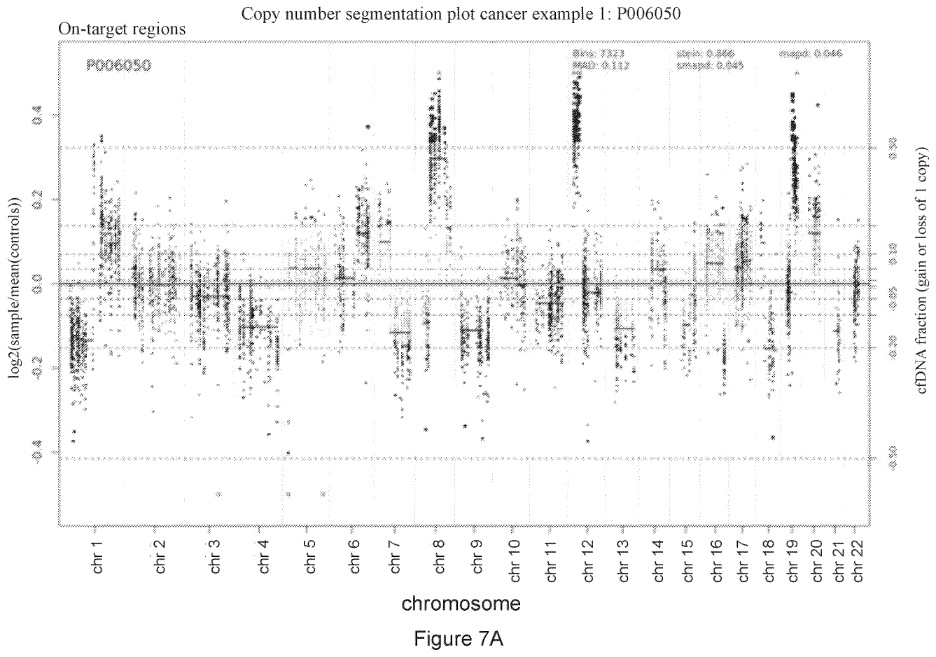

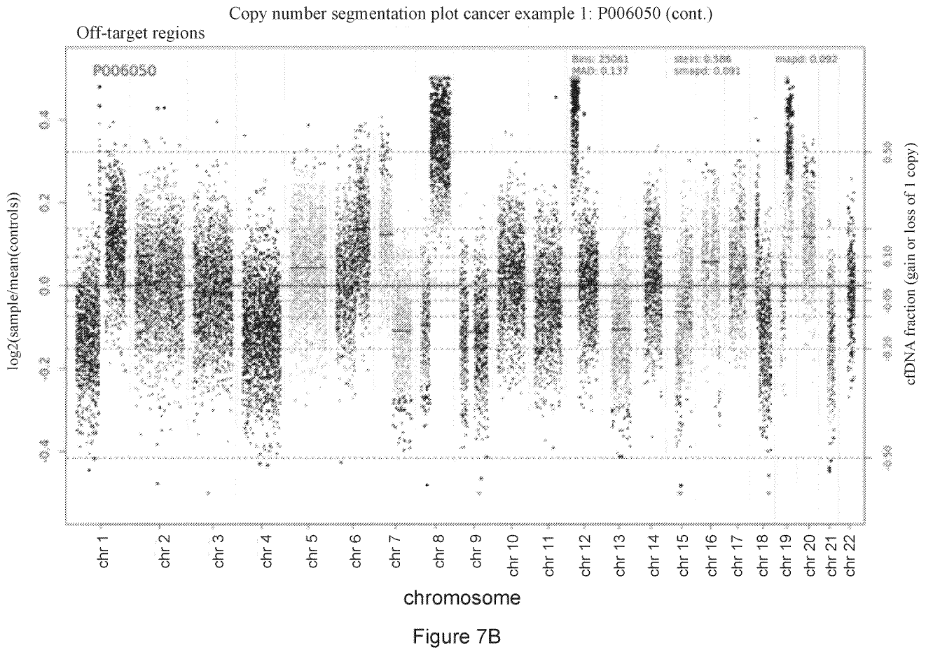

FIG. 7A shows the copy number segmentation using the on-target bin values for a particular cancer subject in the CCGA study—subject P006050. That is, subject P006050 is known to have cancer. The on-target copy number segmentation values, cfDNA fraction (gain or loss of 1 copy) and log₂ of normalized samples (sample/mean (controls)) for each of the chromosome, show aberrant values for chromosomes 8, 12 and 19 for subject P006050. This indicates that the first plurality of bin values (on-target bin values) contain information regarding the cancer state of subject P006060.

FIG. 7B shows the copy number segmentation using the off-target bin values for the same cancer subject as FIG. 7A—subject P006050. Like the on-target copy number segmentation values of FIG. 7A, the off-target copy number segmentation values, cfDNA fraction (gain or loss of 1 copy) and log₂ of normalized samples (sample/mean (controls)) for each of the chromosome, show aberrant values for chromosomes 8, 12 and 19 for subject P006050. This indicates that, like the first plurality of bin values (on-target bin values), the second plurality of bin values (off-target bin values) independently contain information regarding the cancer state of subject P006050. FIGS. 7A and 7B together, are consistent with FIG. 6 in that they show that the on-target regions and off-target regions bear similar signals that can be exploited for disease state (e.g., cancer/non-cancer detection).

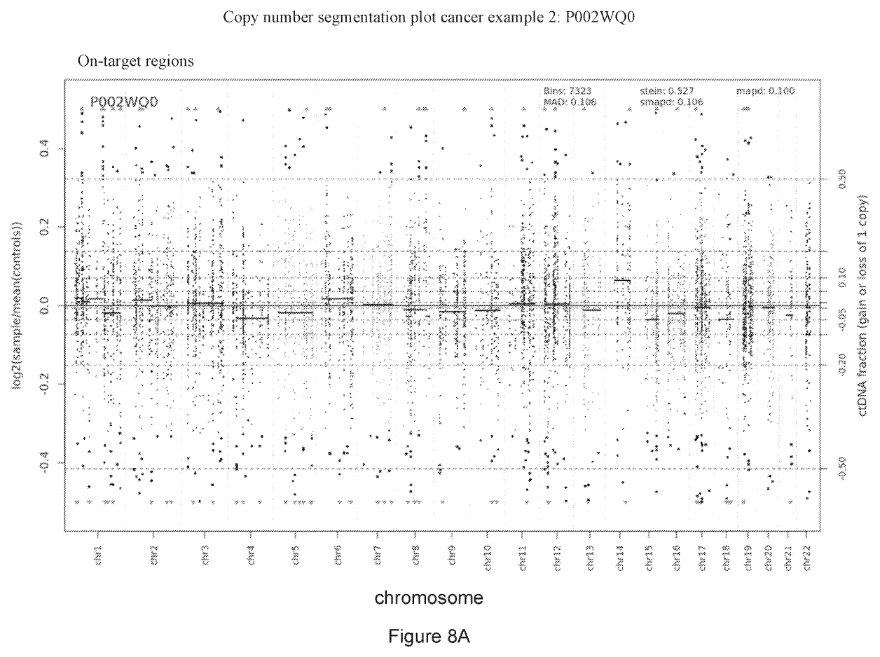

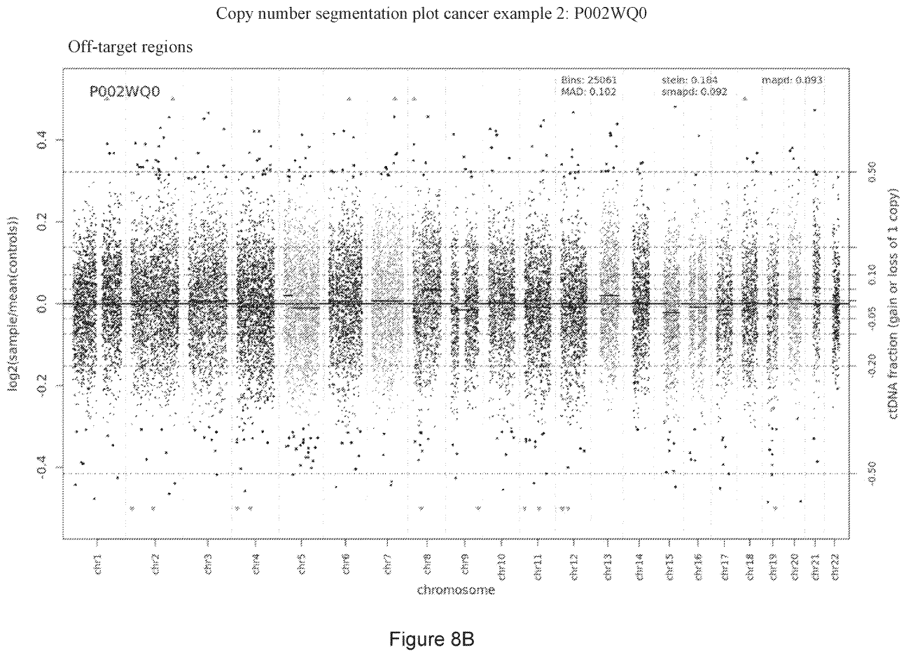

FIG. 8A shows the copy number segmentation using the on-target bin values for a particular cancer subject in the CCGA study—subject P002WQ0. That is, subject P002WQ0 is known to have cancer. The on-target copy number segmentation values, cfDNA fraction (gain or loss of 1 copy) and log₂ of normalized samples (sample/mean (controls)) for each of the chromosome, do not show aberrant values for any of the chromosome for subject P002WQ0. This indicates that the first plurality of bin values (on-target bin values) do contain information regarding the cancer state of subject P002WQ0.

FIG. 8B shows the copy number segmentation using the off-target bin values for the same cancer subject as FIG. 8A—subject P002WQ0. Like the on-target copy number segmentation values of FIG. 8A, the off-target copy number segmentation values, cfDNA fraction (gain or loss of 1 copy) and log₂ of normalized samples (sample/mean (controls)) for each of the chromosome, fail to show aberrant values for any of the chromosomes for subject P002WQ0. This indicates that, although aberrant values were not detected, the first plurality of bin values (on-target bin values) and the second plurality of bin values (off-target bin values) provide consistent information regarding the cancer state of subject P002WQ0.

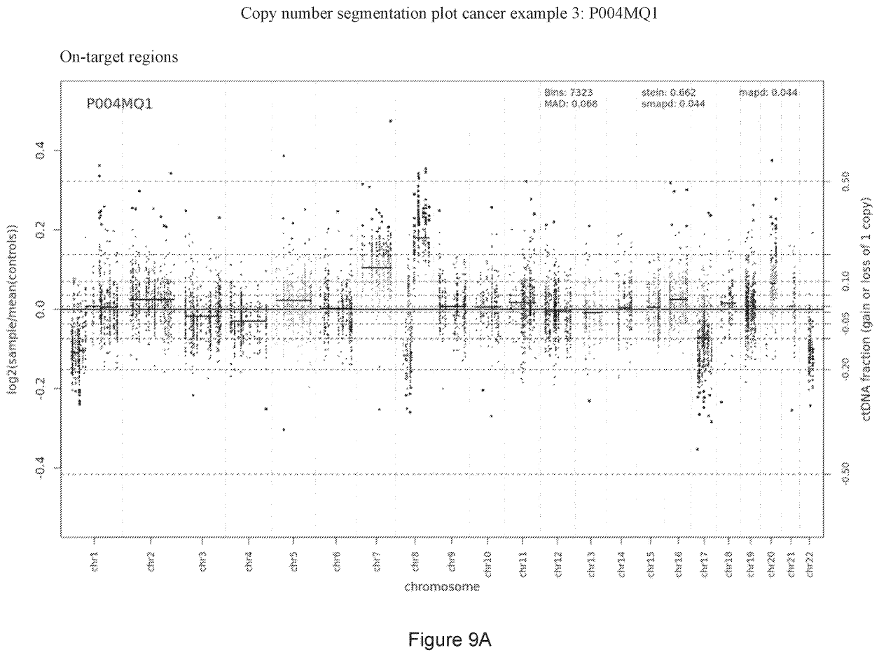

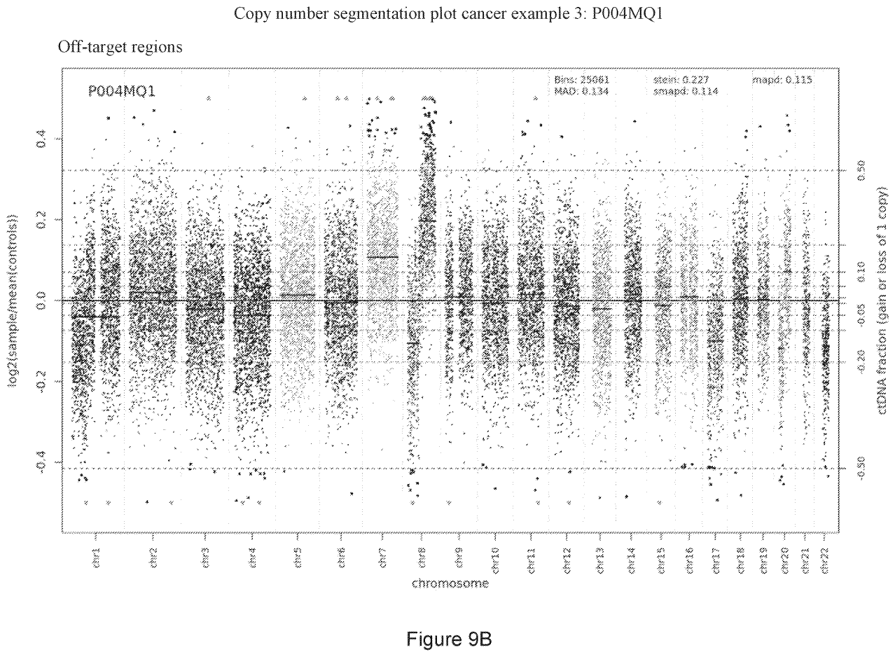

FIG. 9A shows the copy number segmentation using the on-target bin values for a particular cancer subject in the CCGA study-subject P004MQ1. That is, subject P004MQ1 is known to have cancer. The on-target copy number segmentation values, cfDNA fraction (gain or loss of 1 copy) and log₂ of normalized samples (sample/mean (controls)) for each of the chromosome, show aberrant values for chromosomes 7, 8 and 17 for subject P004MQ1. This indicates that the first plurality of bin values (on-target bin values) contain information regarding the cancer state of subject P004MQ1.

FIG. 9B shows the copy number segmentation using the off-target bin values for the same cancer subject as FIG. 9A—subject P004MQ1. Like the on-target copy number segmentation values of FIG. 9A, the off-target copy number segmentation values, cfDNA fraction (gain or loss of 1 copy) and log₂ of normalized samples (sample/mean (controls)) for each of the chromosome, show aberrant values for chromosomes 7, 8 and 17 for subject P004MQ1. This indicates that, like the first plurality of bin values (on-target bin values), the second plurality of bin values (off-target bin values) independently contain information regarding the cancer state of subject P004MQ1. FIGS. 9A and 9B together, are consistent with FIG. 6 in that they show that the on-target regions and off-target regions bear similar signals that can be exploited for disease state (e.g., cancer/non-cancer detection).

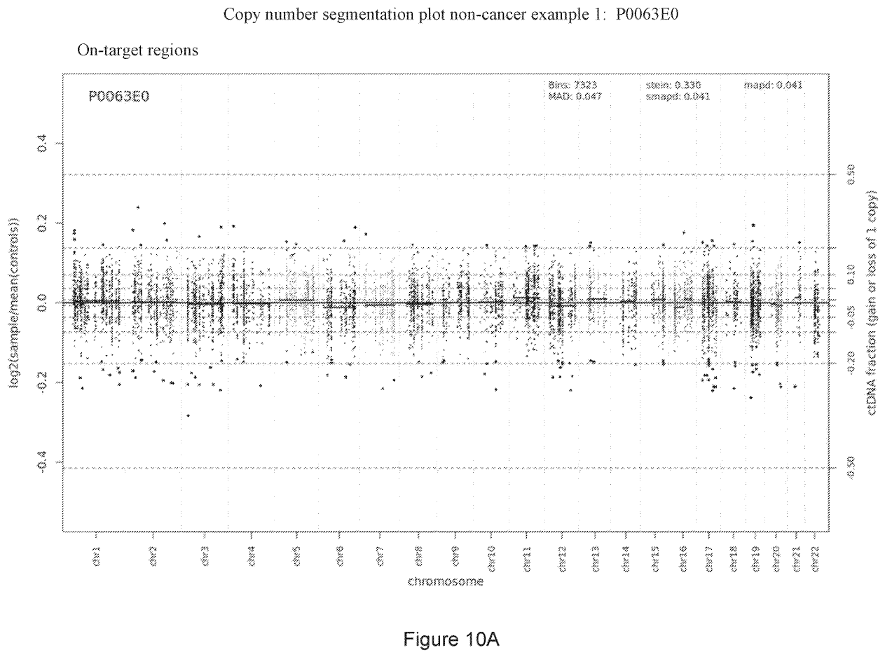

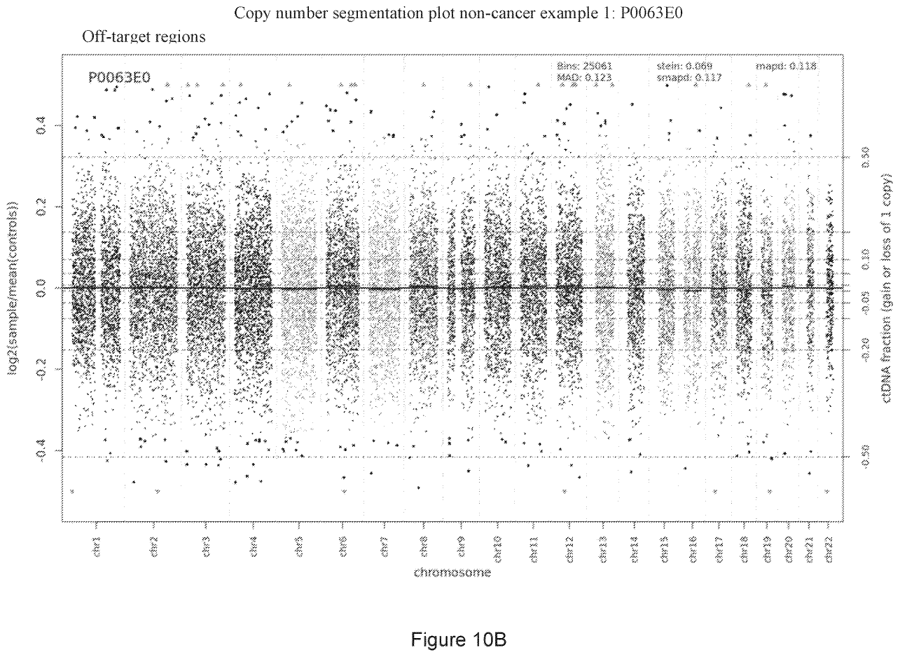

FIG. 10A shows the copy number segmentation using the on-target bin values for a particular subject in the CCGA study—subject P0063EO. That is, subject P0063EO is known to not have cancer. The on-target copy number segmentation values, cfDNA fraction (gain or loss of 1 copy) and log₂ of normalized samples (sample/mean (controls)) for each of the chromosome, do not show aberrant values for any of the chromosomes for subject P0063EO.

FIG. 10B shows the copy number segmentation using the off-target bin values for the same subject as FIG. 10A—subject P0063EO. Like the on-target copy number segmentation values of FIG. 10A, the off-target copy number segmentation values, cfDNA fraction (gain or loss of 1 copy) and log₂ of normalized samples (sample/mean (controls)) for each of the chromosome fail to show aberrant values for any of the chromosomes for subject P0063EO.

The plots in FIGS. 7, 8, 9, and 10 illustrate cfDNA fraction (gain or loss of 1 copy) and log₂ of normalized samples (sample/mean (controls)) for each of the chromosomes. As shown in FIGS. 8-10, the on-target and off-target regions reveal similar patterns of copy number values. While for purposes of showing that the second bins have information content the first and second plurality of bins were subjects to separate sets of dimension reduction components in order to establish that the second plurality of bins (off-target bins) have information content, in some embodiments of the present disclosure, a single set of principal components for the variance exhibited in copy number values of bins in the first and second plurality of bins across a training population is used to train a classifier in accordance with the present disclosure.

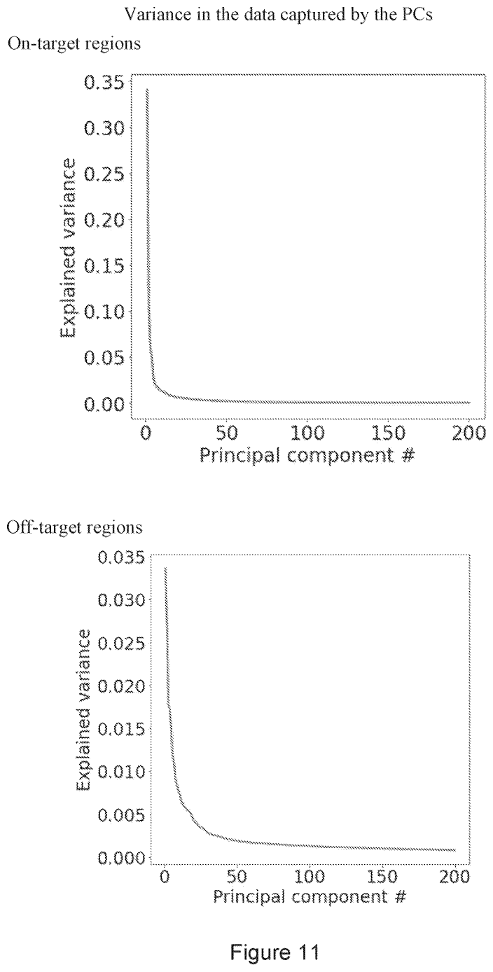

FIG. 11 illustrates explained variance (%) in the data captured when different number of PCs are used, for on-target regions (top panel) and off-target regions (bottom panel). As shown in FIG. 11, for the on-target regions, top several PCs explain most of the variance in the data. The PCs obtained from the off-target regions are less informative but nevertheless a top few PCs are useful features showing the variance in the data. FIG. 11 demonstrates that 5-100 PCs can be used for both on-target and off-target regions.

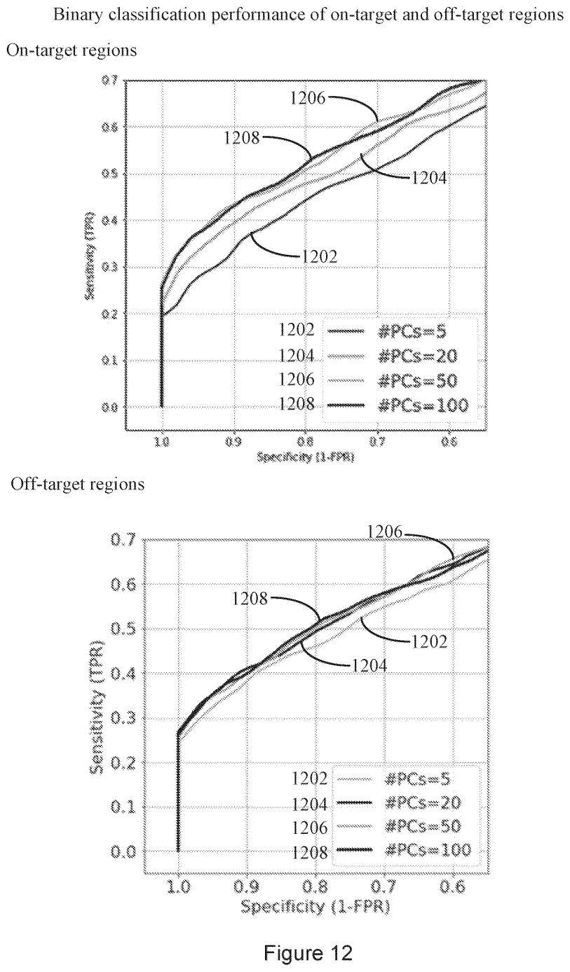

FIGS. 12 to 18 illustrate results of classification performance of a binary logistic regression classifier in accordance with embodiments of the present disclosure, on all analyzed cancers from the CCGA dataset.

FIGS. 12-14 show Receiver Operating Characteristic (ROC) curves (specificity (1-FPR (false positive rate)) versus sensitivity (TPR (true positive rate))), demonstrating classification performance (sensitivity/specificity) of a binary logistic regression classifier in accordance with embodiments of the present disclosure.

In FIG. 12, binary classification performance of a classifier is shown for on-target regions (top panel) or off-target regions (bottom panel), using different numbers of PCs, for all analyzed cancers from the CCGA study. Thus, curve 1202 in the top panel is the performance of a classifier in determining cancer versus no-cancer, trained using the top 5 principal components determined using the first bin values (on-target values) of a training population, for all analyzed cancers from the CCGA study. Curve 1202 in the bottom panel is the binary classification performance of a classifier in determining cancer versus no-cancer, trained using the top 5 principal components determined using the second bin values (off-target values) of a training population, for all analyzed cancers from the CCGA study.

Curve 1204 in the top panel is the binary classification performance of a classifier in determining cancer versus no-cancer, trained using the top 20 principal components determined using the first bin values (on-target values) of a training population, for all analyzed cancers from the CCGA study. Curve 1204 in the bottom panel is the binary classification performance of a classifier in determining cancer versus no-cancer, trained using the top 20 principal components determined using the second bin values (off-target values) of a training population, for all analyzed cancers from the CCGA study.

Curve 1206 in the top panel is the binary classification performance of a classifier in determining cancer versus no-cancer, trained using the top 50 principal components determined using the first bin values (on-target values) of a training population for all analyzed cancers from the CCGA study. Curve 1206 in the bottom panel is the binary classification performance of a classifier in determining cancer versus no-cancer, trained using the top 50 principal components determined using the second bin values (off-target values) of a training population, for all analyzed cancers from the CCGA study.

Curve 1208 in the top panel is the binary classification performance of a classifier in determining cancer versus no-cancer, trained using the top 100 principal components determined using the first bin values (on-target values) of a training population. Curve 1208 in the bottom panel is the performance of a classifier in determining cancer versus no-cancer, trained using the top 100 principal components determined using the second bin values (off-target values) of a training population.

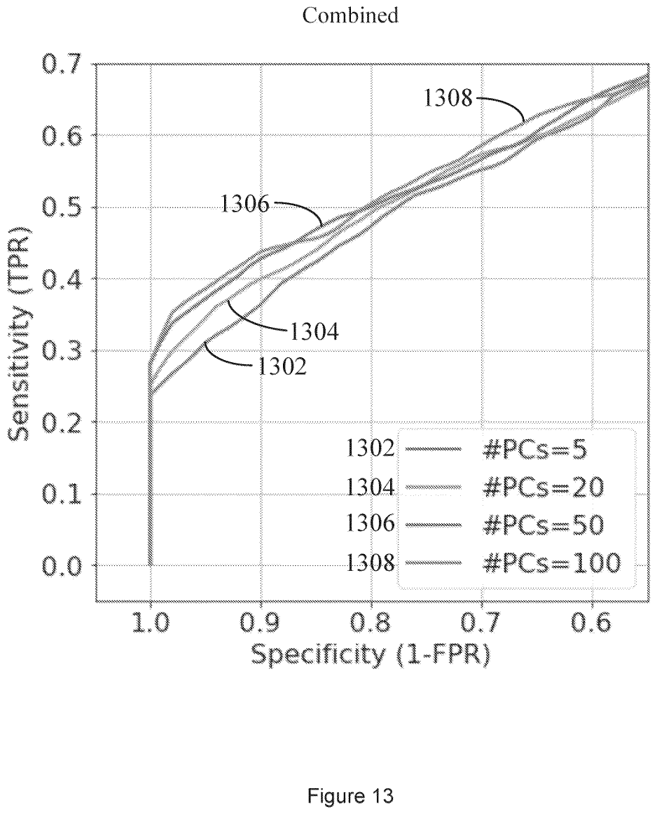

FIG. 13 provides the binary classification performance (sensitivity versus specificity) of a classifier in determining cancer versus no-cancer, trained using the top 5 (curve 1302), 20 (curve 1304), 50 (curve 1306), or 100 (curve 1308) principal components determined across a combination of the first bin values and second bin values of a training population.

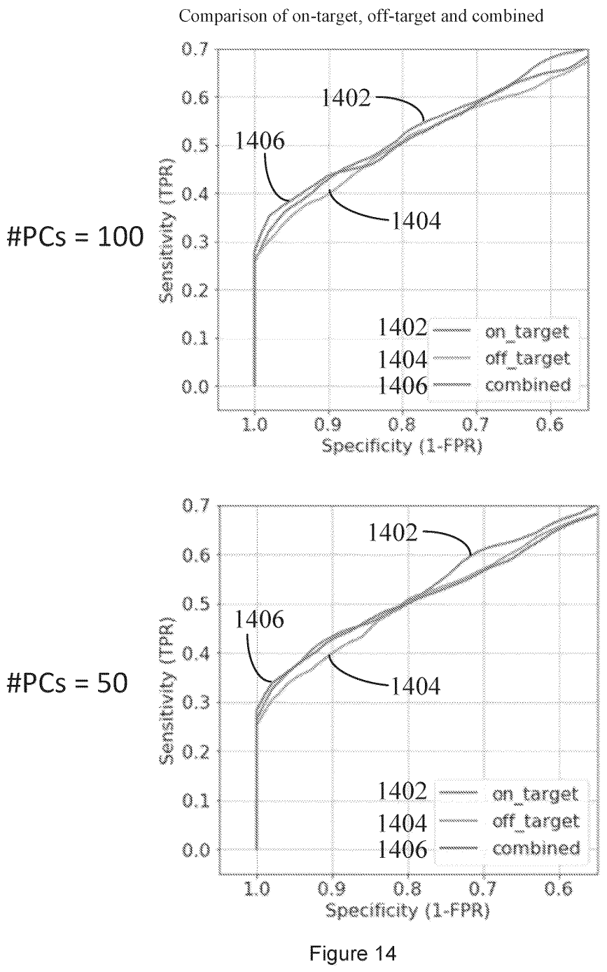

FIG. 14 directly compares the performance of the trained classifiers of FIG. 12 (upper panel, on-target), FIG. 12 (lower panel, off-target) and FIG. 13 (combined on-target and off-target) using 100 principal components (top panel) or 50 principal component (bottom panel) for all subjects of the CCGA dataset. Thus, for FIG. 14 (top panel), the on-target performance (curve 1402) is the binary classification performance of a classifier trained using the variance in bin values of the first plurality (on-target) of bin values across a training population embodied in the top 100 principal components derived for such variance using principal component analysis, for all cancer subjects, regardless of cancer type in the CCGA study. Further, for FIG. 14 (top panel), the off-target performance (curve 1404) is the binary classification performance for a classifier trained using the variance in bin values of the second plurality (off-target) bin values across a training population embodied in the top 100 principal components derived for such variance using principal component analysis, for all cancer subjects, regardless of cancer type in the CCGA study. Further, for FIG. 14 (top panel), the combined-target performance (curve 1406) is the binary classification performance for a classifier trained using the variance in bin values of both the first (on-target) and second (off-target) plurality of bin values across a training population embodied in the top 100 principal components derived for such variance using principal component analysis, for all cancer subjects, regardless of cancer type in the CCGA study. The curves of FIG. 14 (lower panel) are similar, except that the top 50 principal components are used for each respective classifier. The classification performance of the on-target and combined data is similar. FIGS. 12-14 show that about 100 PCs can be useful for both on-target and off-target regions.

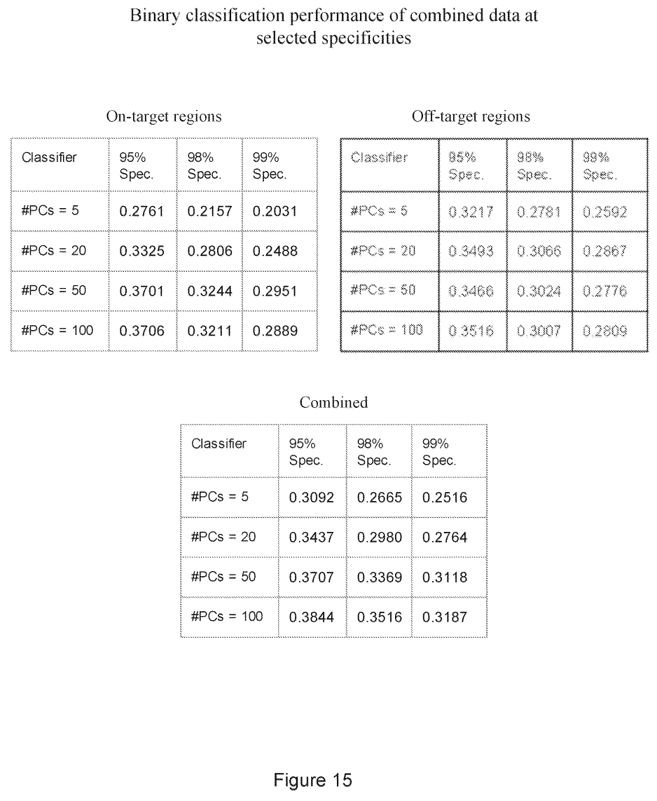

Further, FIG. 15 illustrates results of classification performance of binary logistic regression classifiers using on-target regions (upper left panel), off-target regions (upper right panel), or combined data (lower panel) including both on-target and off-target regions, for 5, 20, 50, and 100 PCs (computed in the manner described above for FIG. 14), and for 95%, 98% and 99% specificities. FIG. 15 shows that, while 5 PCs may be sufficient for classification, using 100 PCs provides additional information.

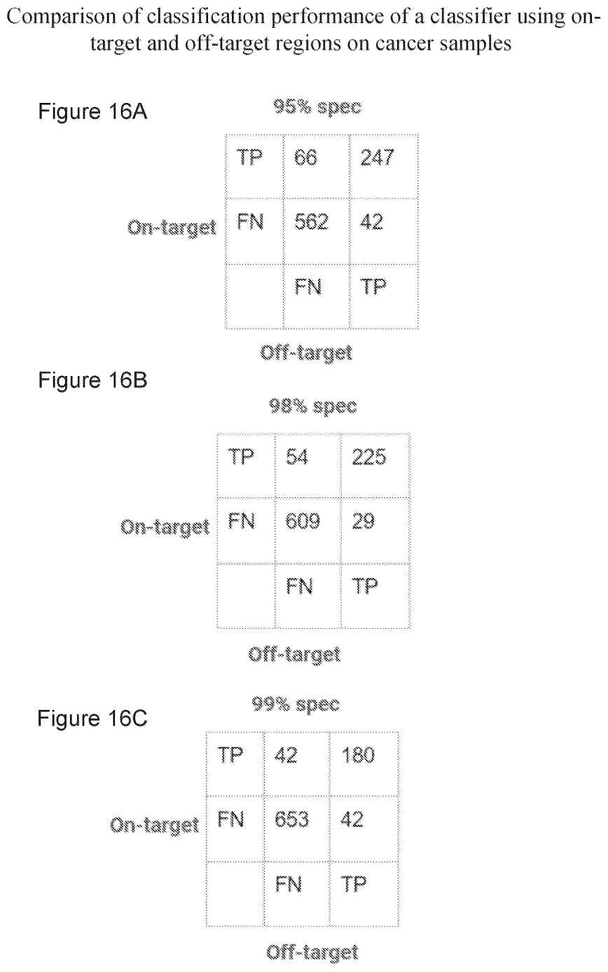

FIGS. 16A, 16B, and 16C illustrate comparison of classification performance of a classifier trained using on-target regions and classifiers trained using off-target regions from all cancer samples from the CCGA dataset, with 95%, 98%, and 99% specificity, respectively. “TP” denotes true positives, and “FN” denotes false negatives.

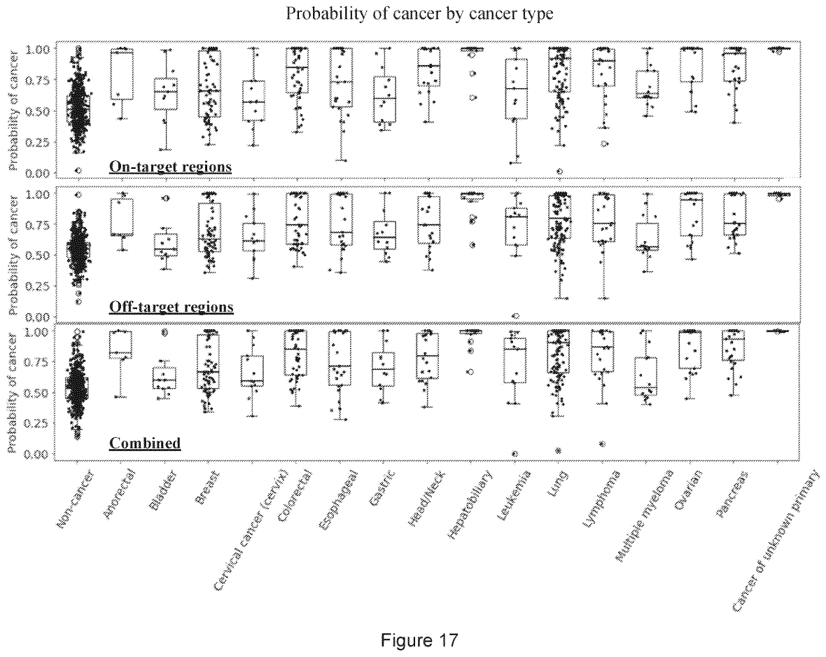

FIG. 17 illustrates results of estimating a probability of cancer by cancer type for samples from the CCGA dataset, using on-target regions, off-target regions, or combined data including both on-target and off-target regions. For FIG. 17 upper panel, a classifier was trained using the bin values of the on-target (first plurality) bins across the CCGA dataset, but the probability of having cancer computed by this classifier was separately evaluated using subjects from the CCGA dataset for each of the designated cancer types. For FIG. 17 middle panel, a classifier was trained using the bin values of the off-target (second plurality) bins across the CCGA dataset, but the probability of having cancer computed by this classifier was separately evaluated using subjects from the CCGA dataset for each of the designated cancer types. For FIG. 17 lower panel, a classifier was trained using the bin values of a combination of both the on-target (first plurality) and off-target (second plurality) bins across the CCGA dataset, but the probability of having cancer computed by this classifier was separately evaluated using subjects from the CCGA dataset for each of the designated cancer types.

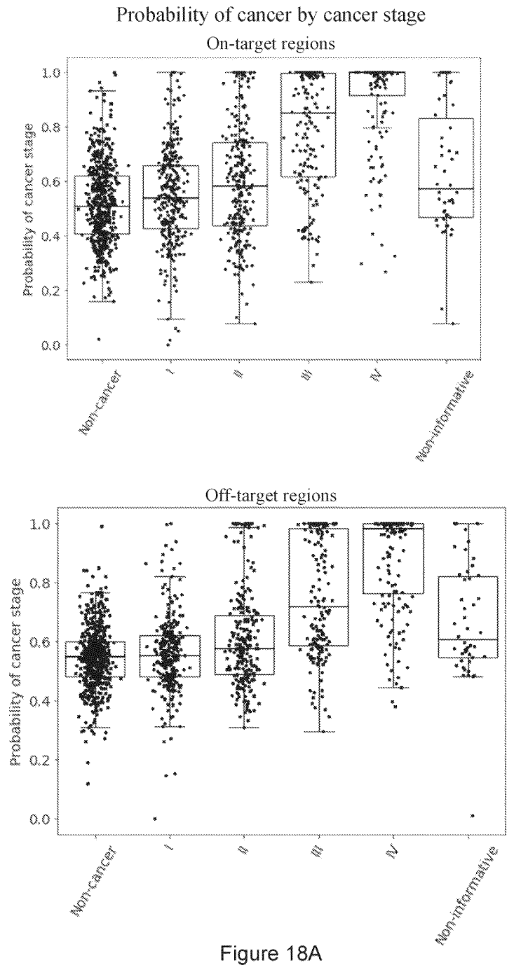

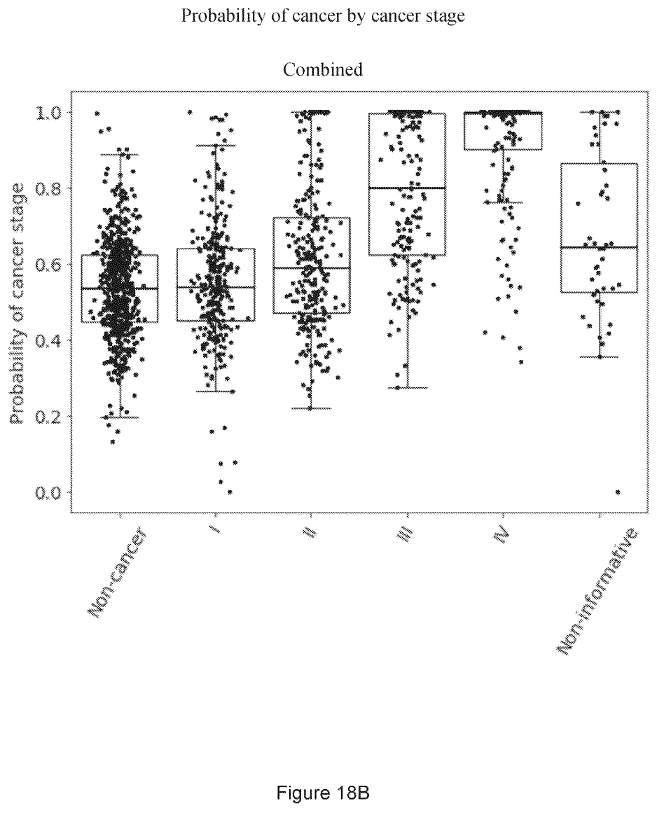

FIG. 18 illustrates results of estimating a probability of cancer by cancer stage for samples from the CCGA dataset, using on-target regions, off-target regions, or combined data including both on-target and off-target regions. The results are shown for non-cancer, cancer stages I, II, III, and IV, and for non-informative estimates. As shown, a classifier that uses information in the on-target regions detects a cancer type with a higher probability than a classifier that uses information in the off-target regions. The classifier trained on the combined data detects a cancer type with a higher probability than a classifier that uses information in the off-target regions. The performance of the classifiers using the on-target regions and combined data is similar.

For FIG. 18 upper-left panel, a classifier was trained using the bin values of the on-target (first plurality) bins across the CCGA dataset, but the probability of having cancer computed by this classifier was separately evaluated for subjects from the CCGA dataset for each stage of cancer, regardless of cancer, ranging from non-cancer to stage IV, as well as for non-informative. For FIG. 18 upper-right panel, a classifier was trained using the bin values of the off-target (second plurality) bins across the CCGA dataset, but the probability of having cancer computed by this classifier was separately evaluated for subjects from the CCGA dataset for each stage of cancer, regardless of cancer, ranging from non-cancer to stage IV, as well as for non-informative. For FIG. 18 lower panel, a classifier was trained using the bin values of a combination of both the on-target (first plurality) and off-target (second plurality) bins across the CCGA dataset, but the probability of having cancer computed by this classifier was separately evaluated using subjects from the CCGA dataset for each stage of cancer, regardless of cancer, ranging from non-cancer to stage IV, as well as for non-informative.

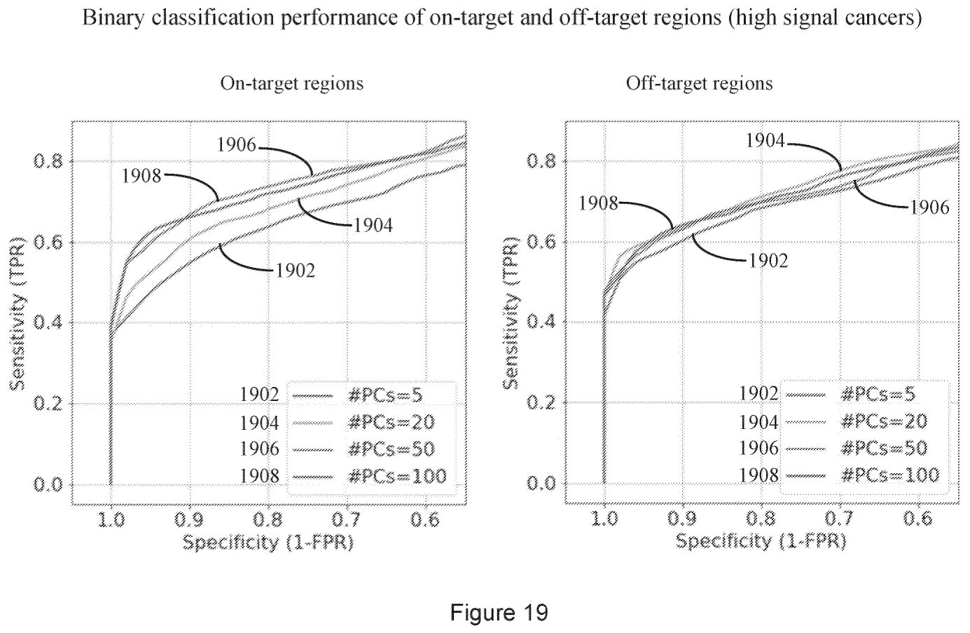

FIGS. 19-25 demonstrate results for high signal cancers from the CCGA dataset.

FIG. 19 illustrates performance of the classifier that uses on-target regions or off-target regions, for different number of PCs. Thus, for FIG. 19 (left panel), the on-target performance is the binary classification performance of a classifier trained using the variance in bin values of the first plurality (on-target) of bin values across a training population embodied in the top 5 (curve 1902), 20 (curve 1904), 50 (curve 1906) or 100 (curve 1908) principal components derived for such variance using principal component analysis, for all high signal cancer subjects in the CCGA study. Further, for FIG. 19 (right panel), the off-target performance is the binary classification performance for a classifier trained using the variance in bin values of the second plurality (off-target) bin values across a training population embodied in the top 5 (curve 1902), 20 (curve 1904), 50 (curve 1906) or 100 (curve 1908) principal components derived for such variance using principal component analysis, for all high signal cancer subjects in the CCGA study.

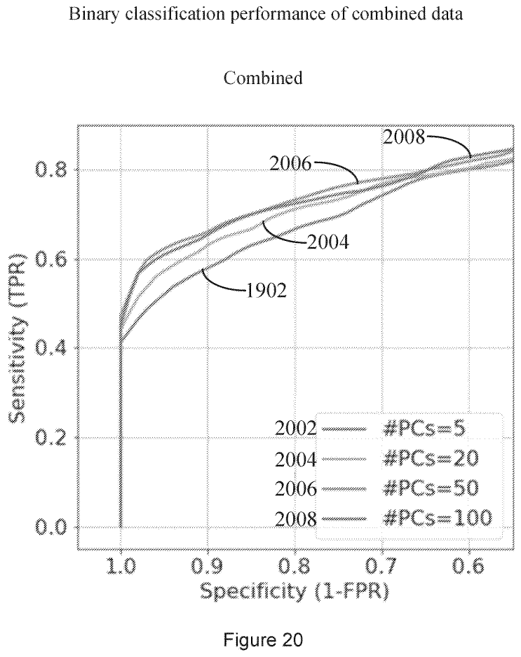

FIG. 20 illustrates the binary classification performance for a classifier trained using the variance in bin values of both the first (on-target) and second (off-target) plurality of bin values across a training population embodied in the top 5, 20, 50 or 100 principal components derived for such variance using principal component analysis, for all high signal cancer subjects in the CCGA study.

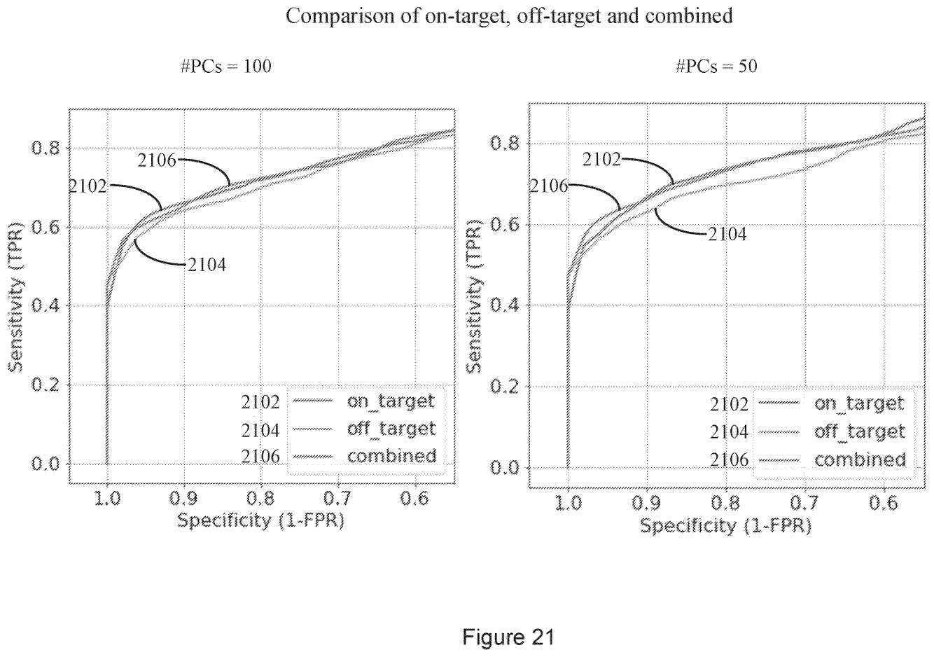

FIG. 21 shows classification performance of a binary logistic regression classifier that uses on-target regions (curve 2102), off-target regions (curve 2104), or combined (curve 2106) data across the subject of the CCGA study including both on-target and off-target regions, for 100 PCs (left panel) and 50 PCs (right panel).

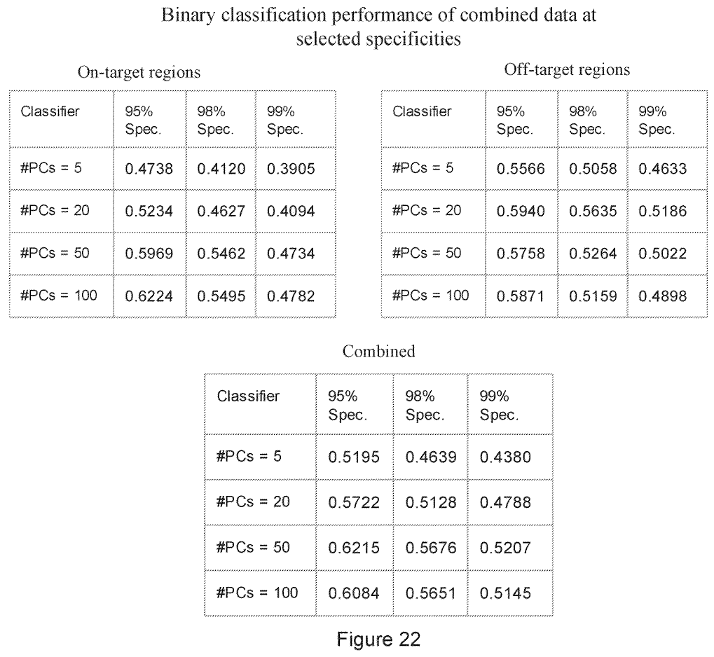

FIG. 22 shows classification performance of a binary logistic regression classifier using on-target regions, off-target regions, or combined data including both on-target and off-target regions across the subject of the CCGA study, for 5, 20, 50, and 100 PCs (top panel) and 50 PCs (bottom panel) and for 95%, 98% and 99% specificities.

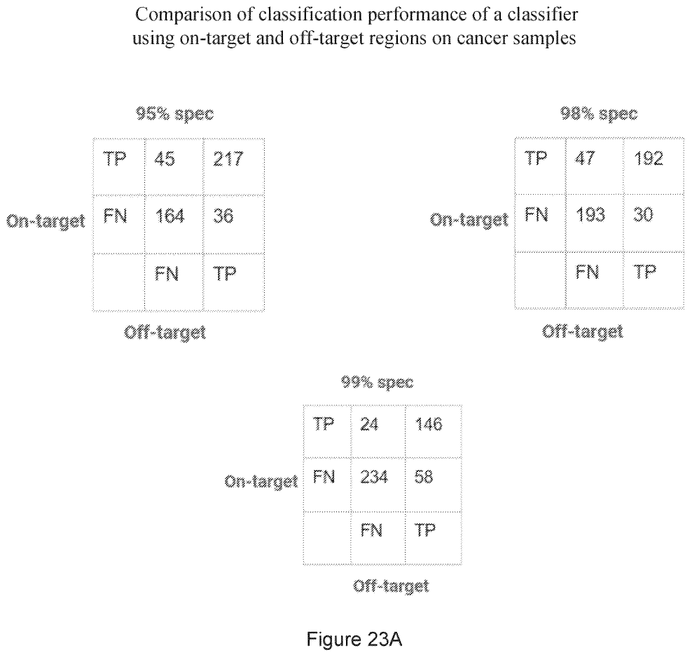

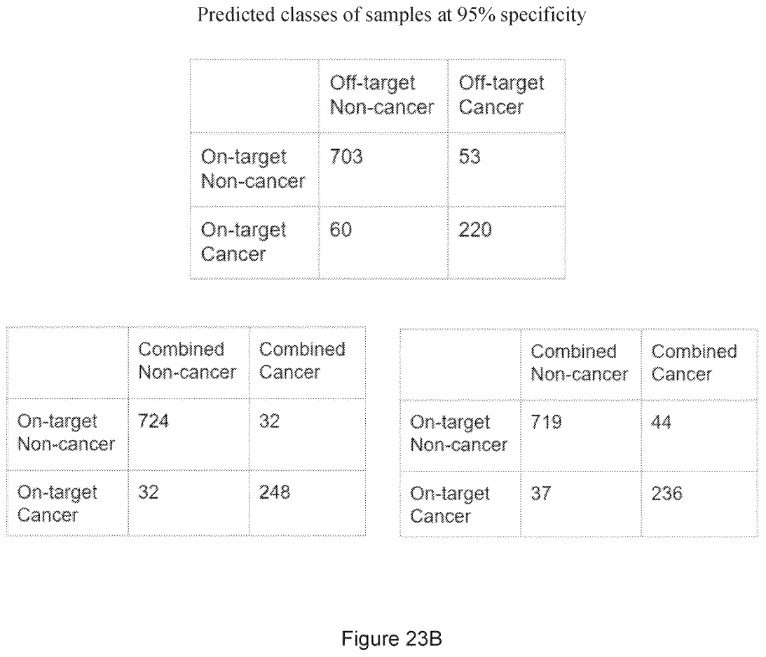

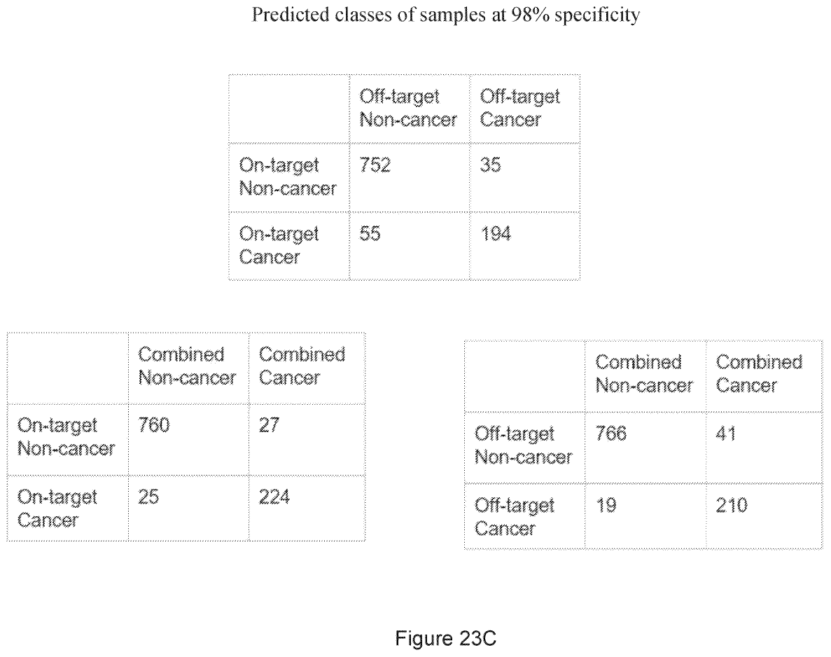

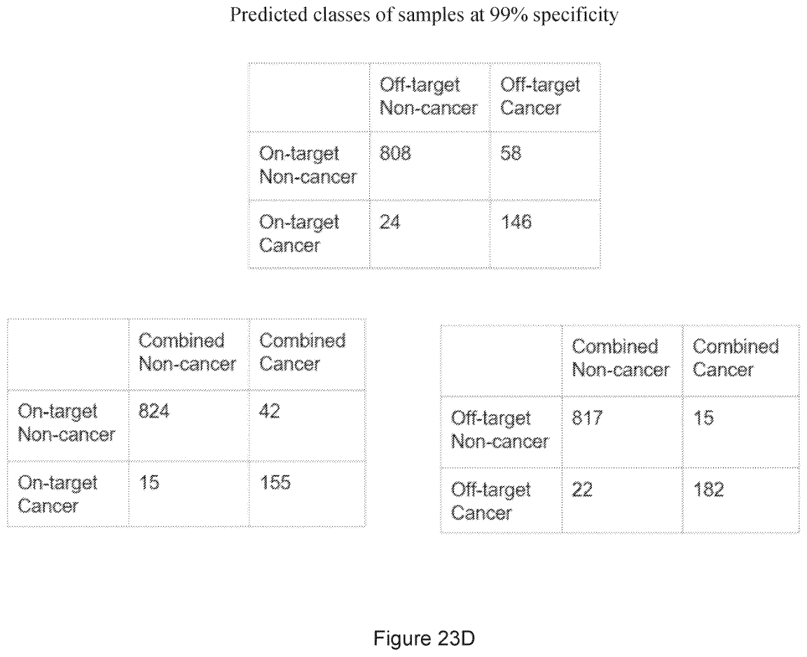

FIGS. 23A, 23B, and 23C illustrate comparison of classification performance of a classifier trained using on-target regions and a classifier trained using off-target regions from high-signal cancer samples from the CCGA dataset, with 95%, 98%, and 99% specificity, respectively. In FIGS. 23A-23C, “TP” denotes true positives, and “FN” denotes false negatives.

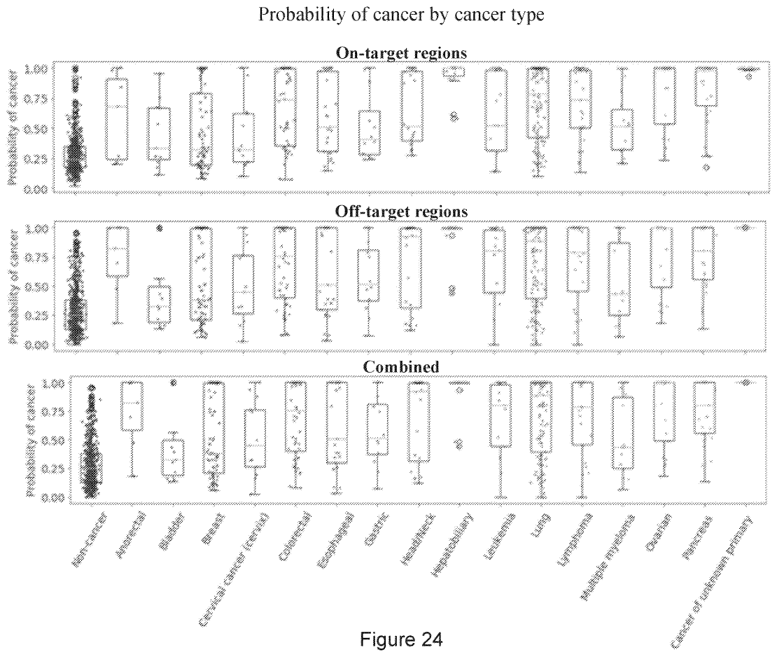

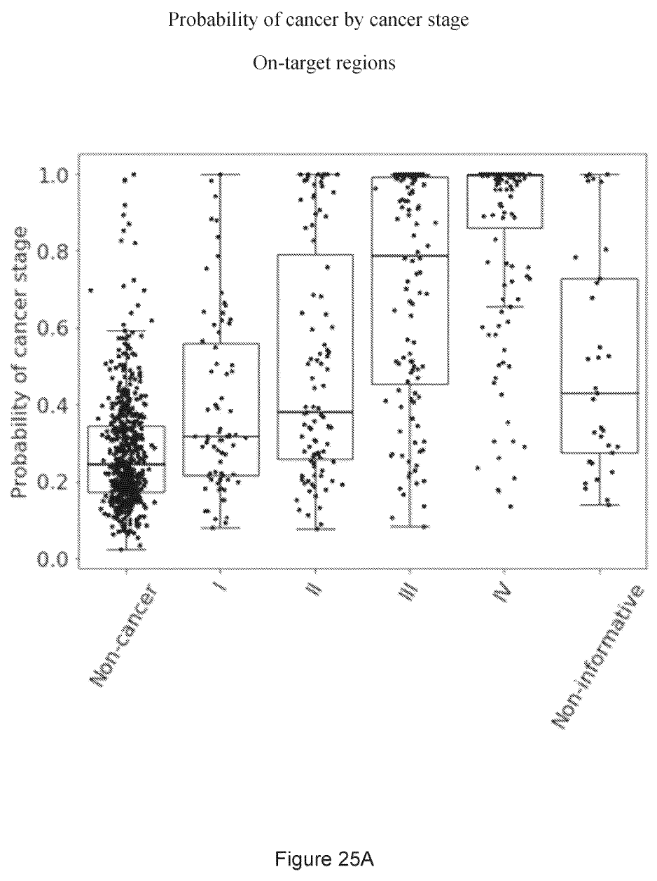

FIG. 24 illustrates results of estimating a probability of cancer by cancer type, using on-target regions, off-target regions, or combined data including both on-target and off-target regions.

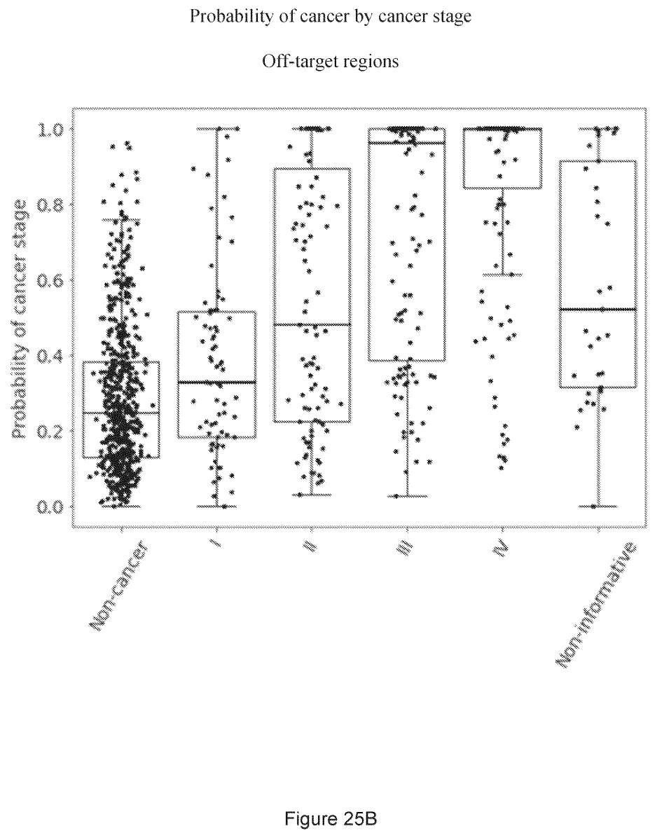

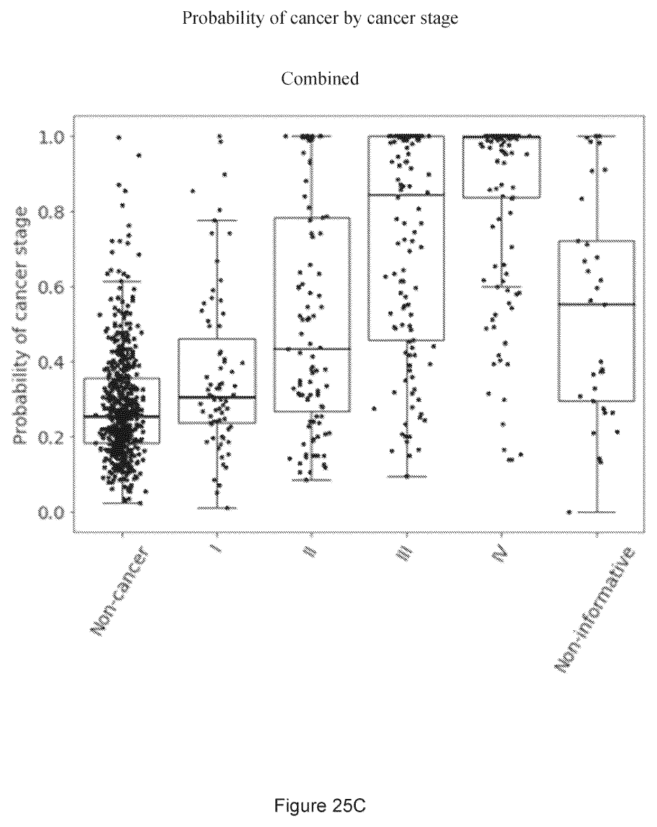

FIG. 25 illustrates results of estimating a probability of cancer by cancer stage, using on-target regions, off-target regions, or combined data including both on-target and off-target regions. The results are shown for non-cancer, cancer stages I, II, III, and IV, and non-informative estimates.

The experiments conducted demonstrate that both on-target and off-target copy number signals can be effectively captured using the CCGA dataset. Some experiments demonstrate that classification performance using on-target data is higher than using off-target data when using all cancer samples and only high-signal cancers. An improvement in classification performance is observed when on-target and off-target data are combined and binary logistic regression is performed on all cancers in the CCGA dataset.

### Example 2—Example Bins for Methylation Embodiments

In some embodiments the first plurality of bins of the present disclosure are designed to encompass targeted regions of the human genome. This example summarizes the identification of suitable regions of the human genome to be encompassed by such bins. Based on the results of Example 2, as further described in Liu et al., “Sensitive and specific multi-cancer detection and localization using methylation signatures in cell-free DNA,” Ann. Oncol 2020, https://doi.org/10.1016/j.annonc.2020.02.011, the portions of the human genome (the hg19 genome, Vogelstin et al., 2013, “Cancer genome landscapes,” Science 339 1546-1558) predicted to contain cancer- and/or tissue-specific methylation patterns in cfDNA relative to non-cancer controls were identified and the most informative regions selected to be represented by the bins of some embodiments of the present disclosure.

Specifically, after bisulfite treatment, targeted cfDNA fragments containing abnormal methylation patterns relative to non-cancer controls from both strands were enriched using biotinylated probes. Briefly, 120-bp biotinylated DNA probes were designed to target enrichment of bisulfite-converted DNA from either hypermethylated fragments (100% methylated CpGs) or hypomethylated fragments (100% unmethylated CpGs); probes tiled target regions with 50% overlap between adjacent probes. A custom algorithm aligned candidate probes to the genome and scored the number of on- and off-target mapping events. Probes with elevated off-target mapping were omitted from the final panel of regions to be represented by the bins of some embodiments of the present disclosure.

As disclosed in U.S. patent application Ser. No. 15/931,022, entitled “Model Based Featurization and Classification,” filed May 13, 2020, a targeted methylation panel, all or a portion of which is represented by the bins of some embodiments of the present disclosure, covering 103,456 distinct regions (17.2 Mb), covering 1,116,720 CpGs was identified using the whole genome bisulfite data obtained from CCGA sub-study CCGA-1. This included 363,033 CpGs in 68,059 regions (7.5 Mb) covered by probes targeting hypomethylated fragments; 585,181 CpGs in 28,521 regions (7.4 Mb) covered by probes targeting hypermethylated fragments; and 218,506 CpGs in 6,876 regions (2.3 Mb) targeting both types of fragments. Individual abnormal target regions contained between 1 and 590 CpGs, with a median CpG count of 3 for hypomethylated target regions and 6 for hypermethylated target regions. CpGs were present in the following genomic regions using the nomenclature of Cavalcante and Sartor, 2017, “annotatr: genomic regions in context,” Bioinformatics33 (15): 2381-2383:193,818 (17%) in the region 1 to 5 kbp upstream of transcription start sites (TSSs); 278,872 (24%) in promoters (<1 kbp upstream of TSSs); 500,996 (43%) in introns; 292,789 (25%) in exons; 247,752 (21%) in intron-exon boundaries (i.e., 200 bp up- or down-stream of any boundary between an exon and intron; boundaries are with respect to the strand of the gene); 134,144 (11%) in 5′-untranslated regions; 28,388 (2.4%) in 3′-untranslated regions; 182,174 (16%) between genes; and the remaining 1,817 (<1%) were not annotated. Percentages were relative to the total number of CpGs and do not sum to 100% because each CpG could receive multiple annotations due to overlapping genes and/or transcripts.

### Example 3—Cancer Assay Probes and Panels

In various embodiments, the predictive classifiers described herein use samples enriched using a cancer assay panel comprising a plurality of probes or a plurality of probe pairs. A number of targeted cancer assay panels include, for example, as described in WO 2019/195268 entitled “Methylation Markers and Targeted Methylation Probe Panels,” filed Apr. 2, 2019, PCT/US2019/053509, filed Sep. 27, 2019 and PCT/US2020/015082 entitled “Detecting Cancer, Cancer Tissue or Origin, or Cancer Type,” filed Jan. 24, 2020 (which are each incorporated by reference herein in their entirety). For example, in some embodiments, the plurality of probes can capture fragments that can together provide information relevant to diagnosis of cancer. In some embodiments, a panel includes at least 50, 100, 500, 1,000, 2,000, 2,500, 5,000, 6,000, 7,500, 10,000, 15,000, 20,000, 25,000, or 50,000 pairs of probes. In other embodiments, a panel includes at least 500, 1,000, 2,000, 5,000, 10,000, 12,000, 15,000, 20,000, 30,000, 40,000, 50,000, or 100,000 probes. The plurality of probes together can comprise at least 0.1 million, 0.2 million, 0.4 million, 0.6 million, 0.8 million, 1 million, 2 million, 3 million, 4 million, 5 million, 6 million, 7 million, 8 million, 9 million, or 10 million nucleotides. The probes (or probe pairs) are specifically designed to target one or more genomic regions differentially methylated in cancer and non-cancer samples. The target genomic regions can be selected to maximize classification accuracy, subject to a size budget (which is determined by sequencing budget and depth of sequencing).

Samples enriched using a cancer assay panel can be subject to targeted sequencing. Samples enriched using the cancer assay panel can be used to detect the presence or absence of cancer generally and/or provide a cancer classification such as cancer type, stage of cancer such as I, II, III, or IV, or provide the tissue of origin where the cancer is believed to originate. Depending on the purpose, a panel can include probes (or probe pairs) targeting genomic regions differentially methylated between general cancerous (pan-cancer) samples and non-cancerous samples, or in cancerous samples with a specific cancer type (e.g., lung cancer-specific targets). Specifically, a cancer assay panel is designed based on bisulfite sequencing data generated from the cell-free DNA (cfDNA) or genomic DNA (gDNA) from cancer and/or non-cancer individuals.

In some embodiments, the cancer assay panel designed by methods provided herein comprises at least 1,000 pairs of probes, each pair of which comprises two probes configured to overlap each other by an overlapping sequence comprising a 30-nucleotide fragment. The 30-nucleotide fragment comprises at least five CpG sites, wherein at least 80% of the at least five CpG sites are either CpG or UpG. The 30-nucleotide fragment is configured to bind to one or more genomic regions in cancerous samples, wherein the one or more genomic regions have at least five methylation sites with an abnormal methylation pattern. Another cancer assay panel comprises at least 2,000 probes, each of which is designed as a hybridization probe complimentary to one or more genomic regions. Each of the genomic regions is selected based on the criteria that it comprises (i) at least 30 nucleotides, and (ii) at least five methylation sites, wherein the at least five methylation sites have an abnormal methylation pattern and are either hypomethylated or hypermethylated.

Each of the probes (or probe pairs) is designed to target one or more target genomic regions. The target genomic regions are selected based on several criteria designed to increase selective enriching of relevant cfDNA fragments while decreasing noise and non-specific bindings. For example, a panel can include probes that can selectively bind and enrich cfDNA fragments that are differentially methylated in cancerous samples. In this case, sequencing of the enriched fragments can provide information relevant to diagnosis of cancer. Furthermore, the probes can be designed to target genomic regions that are determined to have an abnormal methylation pattern and/or hypermethylation or hypomethylation patterns to provide additional selectivity and specificity of the detection. For example, genomic regions can be selected when the genomic regions have a methylation pattern with a low p-value according to a Markov model trained on a set of non-cancerous samples, that additionally cover at least 5 CpG's, 90% of which are either methylated or unmethylated. In other embodiments, genomic regions can be selected utilizing mixture models, as described herein.

Each of the probes (or probe pairs) can target genomic regions comprising at least 25 bp, 30 bp, 35 bp, 40 bp, 45 bp, 50 bp, 60 bp, 70 bp, 80 bp, or 90 bp. The genomic regions can be selected by containing less than 20, 15, 10, 8, or 6 methylation sites. The genomic regions can be selected when at least 80, 85, 90, 92, 95, or 98% of the at least five methylation (e.g., CpG) sites are either methylated or unmethylated in non-cancerous or cancerous samples.

Genomic regions may be further filtered to select those that are likely to be informative based on their methylation patterns, for example, CpG sites that are differentially methylated between cancerous and non-cancerous samples (e.g., abnormally methylated or unmethylated in cancer versus non-cancer). For the selection, calculation can be performed with respect to each CpG site. In some embodiments, a first count is determined that is the number of cancer-containing samples (cancer_count) that include a fragment overlapping that CpG, and a second count is determined that is the number of total samples containing fragments overlapping that CpG (total). Genomic regions can be selected based on criteria positively correlated to the number of cancer-containing samples (cancer_count) that include a fragment overlapping that CpG, and inversely correlated with the number of total samples containing fragments overlapping that CpG (total).

In some embodiments, the number of non-cancerous samples (n_{non-cancer}) and the number of cancerous samples (n_{cancer}) having a fragment overlapping a CpG site are counted. Then the probability that a sample is cancer can be estimated, for example as (n_{cancer}+1)/(n_{cancer}+n_{non-cancer}+2). CpG sites by this metric can be ranked and greedily added to a panel until the panel size budget is exhausted.

Depending on whether the assay is intended to be a pan-cancer assay or a single-cancer assay, or depending on what kind of flexibility is used when picking which CpG sites are contributing to the panel, which samples are used for cancer-count can vary. A panel for diagnosing a specific cancer type (e.g., TOO) can be designed using a similar process. In some embodiments, for each cancer type, and for each CpG site, the information gain is computed to determine whether to include a probe targeting that CpG site. The information gain can be computed for samples with a given cancer type compared to all other samples. For example, two random variables, “AF” and “CT”. “AF” can be a binary variable that indicates whether there is an abnormal fragment overlapping a particular CpG site in a particular sample (yes or no). “CT” can be a binary random variable indicating whether the cancer is of a particular type (e.g., lung cancer or cancer other than lung). One can compute the mutual information with respect to “CT” given “AF.” That is, how many bits of information about the cancer type (lung vs. non-lung in the example) can be gained if one knows whether there is an anomalous fragment overlapping a particular CpG site. This can be used to rank CpG's based on how specific they are for a particular cancer type (e.g., TOO). This procedure can be repeated for a plurality of cancer types. For example, if a particular region is commonly differentially methylated in lung cancer (and not other cancer types or non-cancer), CpG's in that region can have high information gains for lung cancer. For each cancer type, CpG sites ranked by this information gain metric, and then greedily added to a panel until the size budget for that cancer type can be exhausted.

Further filtration can be performed to select target genomic regions that have off-target genomic regions less than a threshold value. For example, a genomic region is selected when there are less than 15, 10 or 8 off-target genomic regions. In other cases, filtration can be performed to remove genomic regions when the sequence of the target genomic regions appears more than 5, 10, 15, 20, 25, or 30 times in a genome. Further filtration can be performed to select target genomic regions when a sequence, 90%, 95%, 98% or 99% homologous to the target genomic regions, appear less than 15, 10 or 8 times in a genome, or to remove target genomic regions when the sequence, 90%, 95%, 98% or 99% homologous to the target genomic regions, appear more than 5, 10, 15, 20, 25, or 30 times in a genome. This can be used to exclude repetitive probes that can pull down off-target fragments, which can impact assay efficiency.

In some embodiments, fragment-probe overlap of at least 45 bp was demonstrated to achieve a non-negligible amount of pulldown (though this number can be different depending on assay details). Furthermore, more than a 10% mismatch rate between the probe and fragment sequences in the region of overlap can be sufficient to greatly disrupt binding, and thus pulldown efficiency. Therefore, sequences that can align to the probe along at least 45 bp with at least a 90% match rate can be candidates for off-target pulldown. Thus, in some embodiments, the number of such regions are scored. The best probes can have a score of 1, showing they match in one place (the intended target region). Probes with a low score (say, less than 5 or 10) can be accepted, but any probes above the score can be discarded. Other cutoff values can be used for specific samples.

In various embodiments, the selected target genomic regions can be located in various positions in a genome, including but not limited to exons, introns, intergenic regions, and other parts. In some embodiments, probes targeting non-human genomic regions, such as those targeting viral genomic regions, can be added.

### CONCLUSION

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A computer-implemented method for determining whether a subject has a condition, the computer-implemented method comprising:
receiving, in electronic form at a computer system, data derived from a biological sample obtained from the subject, the data comprising:
a first set of data corresponding to a first group of nucleic acid regions that are enriched by a plurality of probes used in a targeted sequencing process; and
a second set of data corresponding to a second group of nucleic acid regions, different than the first group of nucleic acid regions, that are not enriched by the plurality of probes and that are derived as a byproduct of the targeted sequencing process;
generating, by the computer system, a set of nucleic acid feature values representative of variation within the biological sample by processing the data from the first and second groups of nucleic acid regions;
applying, by the computer system, a machine-learning model to the set of nucleic acid feature values, the machine-learning model having been trained using reference data derived from biological samples with known conditions; and
generating, based at least in part on output from the trained machine-learning model, an indication of whether the subject has the condition.

2. The computer-implemented method of claim 1, wherein the first group of nucleic acid regions corresponds to on-target regions of a reference genome, and the second group of nucleic acid regions corresponds to off-target regions of the reference genome.

3. The computer-implemented method of claim 1, wherein the targeted sequencing process is targeted DNA methylation sequencing.

4. The computer-implemented method of claim 1, wherein the condition comprises a cancer condition.

5. The computer-implemented method of claim 4, wherein the cancer condition is selected from the group consisting of lung cancer, colorectal cancer, breast cancer, ovarian cancer, pancreatic cancer, liver cancer, prostate cancer, kidney cancer, head and neck cancer, gastric cancer, lymphoma, melanoma, leukemia, and multiple myeloma.

6. The computer-implemented method of claim 1, wherein the generating the set of nucleic acid feature values comprises:
performing a computer-implemented normalization process configured to correct for sequencing bias across the first and second groups of nucleic acid regions.

7. The computer-implemented method of claim 1, wherein the generating the set of nucleic acid feature values comprises:
applying a dimension reduction algorithm to the data to derive a plurality of reduced-dimension values representative of nucleic acid variation across the first and second groups of nucleic acid regions.

8. The computer-implemented method of claim 7, wherein the reduced-dimension values correspond to copy number variation values calculated from normalized sequencing data obtained from both the first and second groups of nucleic acid regions.

9. The computer-implemented method of claim 1, further comprising identifying a stage or a severity of the condition based on the output from the trained machine-learning model.

10. The computer-implemented method of claim 1, further comprising providing an indication of a treatment regimen applicable to the subject based at least in part on the condition.

11. A system, comprising:
one or more processors;
one or more computer readable media storing instructions that are executable by the one or more processors to perform operations including:
receiving, in electronic form at a computer system, data derived from a biological sample obtained from a subject, the data comprising:
a first set of data corresponding to a first group of nucleic acid regions that are enriched by a plurality of probes used in a targeted sequencing process; and
a second set of data corresponding to a second group of nucleic acid regions, different than the first group of nucleic acid regions, that are not enriched by the plurality of probes and that are derived as a byproduct of the targeted sequencing process;
generating, by the computer system, a set of nucleic acid feature values representative of variation within the biological sample by processing the data from the first and second groups of nucleic acid regions;
applying, by the computer system, a machine-learning model to the set of nucleic acid feature values, the machine-learning model having been trained using reference data derived from biological samples with known conditions; and
generating, based at least in part on output from the trained machine-learning model, an indication of whether the subject has the condition.

12. The system of claim 11, wherein the first group of nucleic acid regions corresponds to on-target regions of a reference genome, and the second group of nucleic acid regions corresponds to off-target regions of the reference genome.

13. The system of claim 11, wherein the targeted sequencing process is targeted DNA methylation sequencing.

14. The system of claim 11, wherein the condition comprises a cancer condition.

15. The system of claim 14, wherein the cancer condition is selected from the group consisting of lung cancer, colorectal cancer, breast cancer, ovarian cancer, pancreatic cancer, liver cancer, prostate cancer, kidney cancer, head and neck cancer, gastric cancer, lymphoma, melanoma, leukemia, and multiple myeloma.

16. The system of claim 11, wherein the instructions executable by the one or more processors for generating the set of nucleic acid feature values comprise instructions executable by the one or more processors for:
performing a computer-implemented normalization process configured to correct for sequencing bias across the first and second groups of nucleic acid regions.

17. The system of claim 11, wherein the instructions executable by the one or more processors for generating the set of nucleic acid feature values comprise instructions executable by the one or more processors for:
applying a dimension reduction algorithm to the data to derive a plurality of reduced-dimension values representative of nucleic acid variation across the first and second groups of nucleic acid regions.

18. The system of claim 17, wherein the reduced-dimension values correspond to copy number variation values calculated from normalized sequencing data obtained from both the first and second groups of nucleic acid regions.

19. The system of claim 11, wherein the instructions are further executable by the one or more processors to perform operations including:
identifying a stage or a severity of the condition based on the output from the trained machine-learning model.

20. A non-transitory computer-readable medium storing computer-executable instructions which, when executed by a system, cause the system to perform operations comprising:
receiving, in electronic form at the system, data derived from a biological sample obtained from a subject, the data comprising:
a first set of data corresponding to a first group of nucleic acid regions that are enriched by a plurality of probes used in a targeted sequencing process; and
a second set of data corresponding to a second group of nucleic acid regions, different than the first group of nucleic acid regions, that are not enriched by the plurality of probes and that are derived as a byproduct of the targeted sequencing process;
generating, by the system, a set of nucleic acid feature values representative of variation within the biological sample by processing the data from the first and second groups of nucleic acid regions;
applying, by the system, a machine-learning model to the set of nucleic acid feature values, the machine-learning model having been trained using reference data derived from biological samples with known conditions; and
generating, based at least in part on output from the trained machine-learning model, an indication of whether the subject has the condition.
